SEQUENCE LISTING (51 sequences)

SEQ ID NO: 1 - protein, 420 aa, Homo sapiens
  MOD_RES (116)..(116): Glu or Lys
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 2 - protein, 651 aa, Homo sapiens
  MOD_RES (364)..(364): Glu or Lys
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 3 - protein, 356 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 4 - protein, 563 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 5 - protein, 568 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 6 - protein, 556 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 7 - protein, 473 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 8 - protein, 214 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELKPFITSNNSNPVEDEDAVALTCEPEIQNTTYLWWVNNQSLPVSPRLQLSNDNRTLTLLSVTRNDVGPYECGIQNELSVDHSDPVILNVLYGPDDASGTSPGLSA

SEQ ID NO: 9 - protein, 212 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELKPSISSNNSKPVEDKDAVAFTCEPEAQNTTYLWWVNGQSLPVSPRLQLSNGNRTLTLFNVTRNDARAYVCGIQNSVSANRSDPVTLDVLYGPASGTSPGLSA

SEQ ID NO: 10 - protein, 702 aa, Homo sapiens
  MOD_RES (398)..(398): Glu or Lys
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 11 - protein, 10 aa, Homo sapiens
NIIQNELSVD

SEQ ID NO: 12 - protein, 10 aa, Homo sapiens
NIIQNKLSVD

SEQ ID NO: 13 - protein, 12 aa, Homo sapiens
QNIIQNELSVDH

SEQ ID NO: 14 - protein, 14 aa, Homo sapiens
IQNIIQNELSVDHS

SEQ ID NO: 15 - protein, 12 aa, Homo sapiens
QNIIQNKLSVDH

SEQ ID NO: 16 - protein, 14 aa, Homo sapiens
IQNIIQNKLSVDHS

SEQ ID NO: 17 - DNA, 25 bp, Artificial Sequence
  Description of Artificial Sequence Synthetic primer
cgcatacagtggtcgagagataata

SEQ ID NO: 18 - DNA, 24 bp, Artificial Sequence
  Description of Artificial Sequence Synthetic primer
cgctgtggtcaacacttaatttgt

SEQ ID NO: 19 - DNA, 18 bp, Artificial Sequence
  Description of Artificial Sequence Synthetic probe
atgcatccctgctgatcc

SEQ ID NO: 20 - DNA, 21 bp, Artificial Sequence
  Description of Artificial Sequence Synthetic primer
gaaacccagaacccagtgagt

SEQ ID NO: 21 - DNA, 24 bp, Artificial Sequence
  Description of Artificial Sequence Synthetic primer
gccatagaggacattcaggatgac

SEQ ID NO: 22 - DNA, 16 bp, Artificial Sequence
  Description of Artificial Sequence Synthetic probe
caggcgcagtgattca

SEQ ID NO: 23 - DNA, 40 bp, Artificial Sequence
  Description of Artificial Sequence Synthetic primer
taccgctagcgccaccatggagtctccctcggcccctccc

SEQ ID NO: 24 - DNA, 33 bp, Artificial Sequence
  Description of Artificial Sequence Synthetic primer
gctcgaattctcatatcagagcaaccaaccagc

SEQ ID NO: 25 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 26 - protein, 420 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 27 - protein, 5 aa, Artificial Sequence
  Description of Artificial Sequence Synthetic penta-His tag
HHHHH

SEQ ID NO: 28 - protein, 5 aa, Artificial Sequence
  Description of Artificial Sequence Synthetic peptide
FYFDY

SEQ ID NO: 29 - protein, 10 aa, Artificial Sequence
  Description of Artificial Sequence Synthetic peptide
DXXXXFYFDY

SEQ ID NO: 30 - protein, 6 aa, Artificial Sequence
  Description of Artificial Sequence Synthetic peptide
RFYFDY

SEQ ID NO: 31 - protein, 7 aa, Artificial Sequence
  Description of Artificial Sequence Synthetic peptide
LRFYFDY

SEQ ID NO: 32 - protein, 8 aa, Artificial Sequence
  Description of Artificial Sequence Synthetic peptide
GLRFYFDY

SEQ ID NO: 33 - protein, 9 aa, Artificial Sequence
  Description of Artificial Sequence Synthetic peptide
RGLRFYFDY

SEQ ID NO: 34 - protein, 10 aa, Artificial Sequence
  Description of Artificial Sequence Synthetic peptide
DRGLRFYFDY

SEQ ID NO: 35 - protein, 4 aa, Artificial Sequence
  Description of Artificial Sequence Synthetic peptide
RGLR

SEQ ID NO: 36 - protein, 5 aa, Artificial Sequence
  Description of Artificial Sequence Synthetic peptide
SYWMH

SEQ ID NO: 37 - protein, 18 aa, Artificial Sequence
  Description of Artificial Sequence Synthetic peptide
FIRNKANGGTTEYMSVKG

SEQ ID NO: 38 - protein, 18 aa, Artificial Sequence
  Description of Artificial Sequence Synthetic peptide
FILNKANGGTTEYMSVKG

SEQ ID NO: 39 - protein, 5 aa, Artificial Sequence
  Description of Artificial Sequence Synthetic peptide
TYAMH

SEQ ID NO: 40 - protein, 17 aa, Artificial Sequence
  Description of Artificial Sequence Synthetic peptide
LISNDGSNKYYADSVKG

SEQ ID NO: 41 - protein, 14 aa, Artificial Sequence
  Description of Artificial Sequence Synthetic peptide
TLRRGINVGAYSIY

SEQ ID NO: 42 - protein, 11 aa, Artificial Sequence
  Description of Artificial Sequence Synthetic peptide
YKSDSDKQQGS

SEQ ID NO: 43 - protein, 10 aa, Artificial Sequence
  Description of Artificial Sequence Synthetic peptide
MIWHSGASAV

SEQ ID NO: 44 - protein, 19 aa, Artificial Sequence
  Description of Artificial Sequence Synthetic peptide
FILNKANGGTTEYAASVKG

SEQ ID NO: 45 - DNA, 1506 bp, Artificial Sequence
  Description of Artificial Sequence Synthetic A240 VL bispecific single chain antibody polynucleotide
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtctgggggaggcttggtccagcctgggaggtccctgagactctcctgtgcagcgtctggattcaccgtcagtagctactggatgcactgggtccgccaagctccagggaaggggctggaatgggtaggtttcattagaaacaaagctaatggtgggacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctcatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 46 - protein, 501 aa, Artificial Sequence
  Description of Artificial Sequence Synthetic A240 VL bispecific single chain antibody polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 47 - protein, 19 aa, Artificial Sequence
  Description of Artificial Sequence Synthetic peptide
FIRNKANGGTTEYAASVKG

SEQ ID NO: 48 - protein, 116 aa, Artificial Sequence
  Description of Artificial Sequence Synthetic polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 49 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 50 - protein, 243 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 51 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

(19) United States
(12) Patent Application Publication
(10) Pub. No.: US 2013/0035249 A1
(43) Pub. Date: Feb. 7, 2013

(54) METHODS AND COMPOSITIONS FOR DETECTING AND TREATING CEA-EXPRESSING CANCERS

(71) Applicants: Scott Hammond, Olney, MD (US); Michael David Oberst, Chevy Chase, MD (US); Li Peng, Gaithersburg, MD (US); Jiaqi Huang, Potomac, MD (US)

(72) Inventors: Scott Hammond, Olney, MD (US); Michael David Oberst, Chevy Chase, MD (US); Li Peng, Gaithersburg, MD (US); Jiaqi Huang, Potomac, MD (US)

(73) Assignee: MEDIMMUNE, LLC, Gaithersburg, MD (US)

(21) Appl. No.: 13/513,060

(22) PCT Filed: Nov. 29, 2010
(86) PCT No.: PCT/US10/58206

§ 371 Date: Oct. 17, 2012

Related U.S. Application Data
(60) Provisional application No. 61/265,580, filed on Dec. 1, 2009.

(51) Int. Cl. G01N 33/574; C40B 30/04; C07K 14/47; G01N 33/577

(52) U.S. Cl. 506/9; 435/7.23; 530/350

(57) ABSTRACT

Provided are improved methods and compositions for detecting, monitoring and/or treating a CEA-expressing cancer.

### RELATED APPLICATIONS

This application claims priority to application Ser. No. 61/265,580, filed Dec. 1, 2009. The specification of the foregoing application is hereby incorporated by reference in its entirety.

### CROSS REFERENCE TO SEQUENCE LISTING

The instant application contains a Sequence Listing which has been submitted in ASCII format via EFS-Web and is hereby incorporated by reference in its entirety. Said ASCII copy, created on Nov. 24, 2010, is named MED0526P.txt and is 71,511 bytes in size.

### BACKGROUND OF THE DISCLOSURE

Carcinoembryonic antigen (CEA) is a glycosylated human oncofetal antigen that belongs to the CEA-related cell adhesion molecule (CEACAM) family of the immunoglobulin gene superfamily. CEA has been suggested to mediate cell-cell adhesion, facilitate bacterial colonization of the intestine, and protect the colon from microbial infection by binding and trapping infectious microorganisms. Carcinoembryonic antigen (CEA) is a well-characterized tumor-associated antigen that is frequently over-expressed in human carcinomas and melanomas.

### SUMMARY OF THE DISCLOSURE

The disclosure provides improved methods and compositions for detecting, monitoring and/or treating CEA expressing cancers.

In one aspect, the disclosure provides a method of detecting recurrence of a carcinoembryonic antigen (CEA) expressing cancer. The method involves obtaining a sample from a subject previously diagnosed with and treated for a carcinoembryonic antigen (CEA) expressing cancer. For example, the method involves obtaining a sample from a human subject (e.g., a human patient) previously diagnosed with and treated for cancer that expresses human carcinoembryonic antigen (CEA). The method may comprise a diagnostic step of detecting in said sample a concentration of full-length CEA protein using an antibody, an antigen binding fragment or an immunoglobulin-like molecule (e.g., a diagnostic reagent) that immunospecifically binds to full-length CEA protein but does not immunospecifically bind to short form CEA protein, thereby detecting the concentration of full-length CEA protein without detecting the concentration of short form CEA protein in said sample. Detecting a concentration of full-length CEA protein in said sample above a range observed after treatment indicates recurrence of said CEA expressing cancer.

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM1, 3, 4, 6, 7, and 8). Thus, in certain embodiments, the diagnostic reagent is not a pan-CEA family member antibody, but is specific for full-length CEA protein.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of detecting recurrence of a carcinoembryonic antigen (CEA) expressing cancer. The method includes obtaining a first sample from a subject having a carcinoembryonic antigen (CEA) expressing cancer, wherein said first sample is obtained prior to treatment. For example, the method involves obtaining a sample from a human subject (e.g., a human patient) diagnosed with a cancer that expresses human carcinoembryonic antigen (CEA) prior to the beginning of treatment. The method may comprise a diagnostic step of detecting in said first sample a pre-treatment concentration of full-length CEA protein using an antibody, an antigen binding fragment or an immunoglobulin-like molecule (e.g., a diagnostic reagent) that immunospecifically binds to full-length CEA protein but does not immunospecifically bind to short form CEA protein, thereby detecting the concentration of full-length CEA protein without detecting the concentration of short form CEA protein in said first sample. The method may include obtaining a second sample from said subject, and detecting in said second sample a concentration of full-length CEA protein using said antibody, antigen binding fragment or immunoglobulin-like molecule (e.g., using said diagnostic reagent), thereby detecting the concentration of full-length CEA protein without detecting the concentration of short form CEA protein in said second sample. For example, the second sample may be collected at some period of time (or multiple time points) after initiation of treatment, so that decrease in CEA as a result of treatment can be measured. The method may include obtaining one or more further samples from said subject at a time later than that for obtaining said second sample, and detecting in said one or more further samples a concentration of full-length CEA protein using said antibody, antigen binding fragment or immunoglobulin-like molecule (e.g., said diagnostic reagent), thereby detecting the concentration of full-length CEA protein without detecting the concentration of short form CEA protein in said one or more further samples. Detecting a concentration of full-length CEA protein in said one or more further samples above the concentration of full-length CEA protein observed in said second sample indicates recurrence of said CEA expressing cancer. By way of example, after successful treatment, CEA levels will decrease. Following that decrease, the patient can be monitored, and an increase in CEA levels following this decrease may be indicative of recurrence of the cancer. In certain embodiments, an increase in CEA levels to approximately the same or greater concentration than that observed in the pre-treatment sample is indicative of recurrence of the CEA expressing cancer.

In certain embodiments, the initial treatment comprises surgery, and the first sample is taken prior to surgical resection of all or a portion of the CEA-expressing tumor. In certain embodiments, the initial treatment comprises one or more of surgery, chemotherapy, radiation therapy, immunotherapy, or a biological therapy, such as a monoclonal antibody therapy, gene therapy, oncolytic therapy, or viral therapy. In certain embodiments, treatment is ongoing, such that the first sample is taken prior to the commencement of any treatment, but the second and/or further samples are taken during a cycle of treatment (e.g., during a cycle of chemotherapy or radiation treatment).

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM 1, 3, 4, 6, 7, and 8). Thus, in certain embodiments, the diagnostic reagent is not a pan-CEA family member antibody, but is specific for full-length CEA protein.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagents is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of determining susceptibility to anti-carcinoembryonic antigen (CEA) cancer therapy. The method includes detecting a concentration of full-length CEA protein in a sample from a subject using an antibody, an antigen binding fragment or an immunoglobulin-like molecule (e.g., a diagnostic reagent) that immunospecifically binds to full-length CEA protein but does not immunospecifically bind to short form CEA protein, thereby detecting the concentration of full-length CEA protein without detecting the concentration of short form CEA protein in said sample. The method may include comparing said concentration of full-length CEA protein to a standard range reflecting full-length CEA protein concentration in samples from healthy subjects. Detecting a concentration of full-length CEA protein above said standard range indicates susceptibility to anti-CEA cancer therapy.

In certain of the foregoing and following embodiments, the standard range reflecting full-length CEA protein concentration in samples from healthy subject is less than or equal to 3 ug/L (3 ng/mL) in serum of non-smokers and less than or equal to 5 μg/L (5 ng/mL) in serum of smokers. In certain embodiments, the standard range is less than or equal to 5 μg/L (5 ng/mL) in serum, regardless of smoking status.

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM 1, 3, 4, 6, 7, and 8). Thus, in certain embodiments, the diagnostic reagent is not a pan-CEA family member antibody, but is specific for full-length CEA protein.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagents is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of monitoring anti-carcinoembryonic antigen (CEA) cancer therapy. The method includes detecting a concentration of full-length CEA protein in a sample from a subject undergoing treatment for a CEA expressing cancer using an antibody, an antigen binding fragment or an immunoglobulin-like molecule (e.g., a diagnostic reagent) that immunospecifically binds to full-length CEA protein but does not immunospecifically bind to short form CEA protein, thereby detecting the concentration of full-length CEA protein without detecting the concentration of short form CEA protein in said sample. The method may include comparing said concentration of full-length CEA protein to a concentration of full-length CEA protein in a sample from said same subject, which sample was obtained prior to said treatment or at an earlier time point during said treatment. A decrease in full-length CEA concentration in a sample obtained at a later point during treatment or after conclusion of treatment versus that obtained prior to treatment or at an earlier time point during said treatment indicates effectiveness of said treatment, thereby monitoring said anti-CEA cancer therapy.

In certain embodiments, the initial treatment comprises surgery, and the first sample is taken prior to surgical resection of all or a portion of the CEA-expressing tumor. In certain embodiments, the initial treatment comprises one or more of surgery, chemotherapy, radiation therapy, immunotherapy, or a biological therapy, such as a monoclonal antibody therapy, gene therapy, oncolytic therapy, or viral therapy. In certain embodiments, treatment is ongoing, such that the first sample is taken prior to the commencement of any treatment, but the second and/or further samples are taken during a cycle of treatment (e.g., during a cycle of chemotherapy or radiation treatment).

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM 1, 3, 4, 6, 7, and 8). Thus, in certain embodiments, the diagnostic reagent is not a pan-CEA family member antibody, but is specific for full-length CEA protein.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagents is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of treating a subject having a carcinoembryonic antigen (CEA) expressing cancer. The method involves obtaining a sample from a subject prior to treatment for a carcinoembryonic antigen (CEA) expressing cancer, such as prior to initiation of treatment. The method may include detecting in said sample (the initial sample) a concentration of full-length CEA protein using an antibody, an antigen binding fragment or an immunoglobulin-like molecule (e.g., a diagnostic reagent) that immunospecifically binds to full-length CEA protein but does not immunospecifically bind to short form CEA protein, thereby detecting the concentration of full-length CEA protein without detecting the concentration of short form CEA protein in said sample. The method may include comparing said concentration of full-length CEA protein to a standard range reflecting full-length CEA protein concentration in samples from healthy subjects, wherein detecting the concentration of full-length CEA protein above said standard range indicates susceptibility to anti-CEA cancer therapy. The method may include treating said subject who, based on the initial diagnostic testing is determined to be susceptible to anti-CEA cancer therapy, with an anti-CEA cancer therapeutic. The method may include detecting a concentration of full-length CEA protein in a post-treatment sample from said subject using an antibody, an antigen binding fragment or an immunoglobulin-like molecule that immunospecifically (e.g., a diagnostic reagent) binds to full-length CEA protein but does not immunospecifically bind to a short form CEA protein, thereby detecting the concentration of full-length CEA protein without detecting the concentration of short form CEA protein in said post-treatment sample. The method may include comparing said concentration of full-length CEA protein in said post-treatment sample to said concentration in the sample obtained prior to treatment. A decrease in full-length CEA protein concentration in said post-treatment sample relative to said pre-treatment sample indicates the effectiveness of said anti-CEA cancer therapeutic in said method of treating said subject.

In certain embodiments, treatment includes an anti-CEA cancer therapeutic along with one or more additional treatment modalities. Exemplary treatment modalities include, but are not limited to surgery, chemotherapy, radiation therapy, immunotherapy, biological therapies such as monoclonal antibodies and gene therapy, herbal therapy, acupuncture, or dietary therapy. In certain embodiments, treatment is ongoing, such that the first sample is taken prior to the commencement of any treatment, but the second and/or further samples are taken during a cycle of treatment (e.g., during a cycle of chemotherapy or radiation treatment).

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM 1, 3, 4, 6, 7, and 8). Thus, in certain embodiments, the diagnostic reagent is not a pan-CEA family member antibody, but is specific for full-length CEA protein.

In certain embodiments, the diagnostic reagent and the anti-CEA therapeutic bind to the same or substantially the same epitope of CEA.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of determining susceptibility to a cancer therapeutic that immunospecifically binds to carcinoembryonic antigen (CEA) protein. The method comprises selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein. The method may include detecting a concentration of said target CEA protein in a sample from said subject using an antibody, an antigen binding fragment or an immunoglobulin-like molecule (e.g., a diagnostic reagent) that immunospecifically binds to an epitope on said target CEA protein that is the same or substantially the same as the epitope that said cancer therapeutic immunospecifically binds, thereby detecting the concentration of the target CEA protein without detecting the concentration of other non-target forms of CEA protein in said sample. The method may include comparing said concentration of said target CEA protein to a standard range reflecting target CEA protein concentration in samples from healthy subjects. Detecting a concentration of said target CEA protein in said sample above said standard range indicates susceptibility to said cancer therapeutic.

In certain embodiments, the target CEA protein is full-length CEA protein. In certain embodiments, the target CEA protein is short form CEA protein. In either case, it is envisioned that a diagnostic reagent that immunospecifically binds a target CEA protein will immunospecifically bind both soluble and cell associated target CEA protein, both of which are mature CEA protein. Such a diagnostic reagent may also bind to the pro-form of target CEA protein. However, given that the diagnostically and therapeutically relevant CEA is the mature CEA protein expressed on tumors and present in bodily fluids, the relevant reagents are those that immunospecifically bind to mature target CEA.

In certain embodiments, the cancer therapeutic and the diagnostic reagent are the same protein. In certain embodiments, the cancer therapeutic and the diagnostic reagent share at least one antigen binding fragment.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of monitoring treatment. The method comprises selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein. The method may comprise detecting a concentration of said target CEA protein in a sample from said subject, which subject is undergoing treatment for a CEA expressing cancer, using an antibody, an antigen binding fragment or an immunoglobulin-like molecule (e.g., a diagnostic reagent) that immunospecifically binds to an epitope on said target CEA protein that is the same or substantially the same as the epitope that said cancer therapeutic immunospecifically binds, thereby detecting the concentration of a target CEA protein without detecting the concentration of other non-target forms of CEA protein in said sample. The method may include comparing said concentration of target CEA protein to a concentration of target CEA protein in an earlier sample from said same subject, which earlier sample was obtained prior to treatment with said cancer therapeutic or at an earlier time point during treatment with said cancer therapeutic. A decrease in target CEA concentration in a sample obtained at a later point during treatment with said cancer therapeutic versus that obtained prior to treatment or at an earlier time point during treatment with said cancer therapeutic indicates effectiveness of said cancer therapeutic, thereby monitoring said treatment.

In certain embodiments, the target CEA protein is full-length CEA protein. In certain embodiments, the target CEA protein is short form CEA protein. In either case, it is envisioned that a diagnostic reagent that immunospecifically binds a target CEA protein will immunospecifically bind both soluble and cell associated target CEA protein. Such a diagnostic reagent may also bind to the pro-form of target CEA protein. However, given that the diagnostically and therapeutically relevant CEA is the mature CEA protein expressed on tumors and present in bodily fluids, the relevant reagents are those that immunospecifically bind to mature target CEA.

In certain embodiments, the cancer therapeutic and the diagnostic reagent are the same protein. In certain embodiments, the cancer therapeutic and the diagnostic reagent share at least one antigen binding fragment.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of treating a subject having carcinoembryonic antigen (CEA) expressing cancer. The method comprises selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein. The method may include detecting a concentration of said target CEA protein in a sample from said subject using an antibody, an antigen binding fragment or an immunoglobulin-like molecule (e.g., a diagnostic reagent) that immunospecifically binds to an epitope on said target CEA protein that is the same or substantially the same as the epitope that said therapeutic immunospecifically binds, thereby detecting the concentration of said target CEA protein without detecting the concentration of other non-target forms of CEA protein in said sample. The method may include comparing said concentration of said target CEA protein to a standard range reflecting target CEA protein concentration in samples from healthy subjects. Detecting a concentration of said target CEA protein above said standard range indicates susceptibility to a cancer therapeutic that immunospecifically binds to target CEA protein. The method may include treating the subject with the cancer therapeutic that immunospecifically binds to target CEA protein if said subject is determined to be susceptible to said cancer therapeutic. The method may include detecting, in a post-treatment sample from said subject undergoing treatment with said cancer therapeutic, a concentration of target CEA protein using an antibody, an antigen binding fragment or an immunoglobulin-like molecule that immunospecifically binds to an epitope on target CEA that is the same or substantially the same as the epitope that said cancer therapeutic immunospecifically binds, thereby detecting the concentration of said target CEA protein without detecting the concentration of other non-target forms of CEA protein in said sample. The method may include comparing said concentration of target CEA protein to a concentration of target CEA protein in a sample from said same subject, which sample was obtained prior to treatment with said cancer therapeutic or at an earlier time point during said treatment, wherein a decrease in target CEA concentration in a sample obtained at a later point during treatment with said cancer therapeutic versus that obtained prior to or at an earlier time point during said treatment indicates effectiveness of said treatment of said subject.

In certain embodiments, the target CEA protein is full-length CEA protein. In certain embodiments, the target CEA protein is short form CEA protein. In either case, it is envisioned that a diagnostic reagent that immunospecifically binds a target CEA protein will immunospecifically bind both soluble and cell associated target CEA protein. Such a diagnostic reagent may also bind to the pro-form of target CEA protein. However, given that the diagnostically and therapeutically relevant CEA is the mature CEA protein expressed on tumors and present in bodily fluids, the relevant reagents are those that immunospecifically bind to mature target CEA.

In certain embodiments, the target CEA is short form CEA and the diagnostic reagent is immunospecific for a short form CEA polymorphism comprising NIIQNELSVD (SEQ ID NO: 11), but is not immunospecific for a short form CEA polymorphism comprising NIIQNKLSVD (SEQ ID NO: 12). In other embodiments, the diagnostic reagent is immunospecific for a short form CEA polymorphism comprising NIIQNKLSVD (SEQ ID NO: 12), and is not immunospecific for a short form CEA polymorphism comprising NIIQNELSVD (SEQ ID NO: 11). In other embodiments, the diagnostic reagent is immunospecific for both of the foregoing short form CEA polymorphisms.

In certain embodiments, the cancer therapeutic and the diagnostic reagent are the same protein. In certain embodiments, the cancer therapeutic and the diagnostic reagent share at least one antigen binding fragment.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method comprising detecting a concentration of full-length CEA protein or RNA and a concentration of short form CEA protein or RNA in a sample from a subject and determining a ratio of full-length CEA protein or RNA concentration to short form CEA protein or RNA concentration.

In certain embodiments, the method comprises comparing said ratio to a standard reflecting the standard ratio of full-length CEA protein or RNA concentration to short form CEA protein or RNA concentration in samples from healthy subjects. A ratio that varies significantly from the standard ratio is indicative of presence of a CEA-expressing cancer.

In certain embodiments, detecting a concentration of full-length CEA protein comprises contacting a sample with an antibody, an antigen binding fragment or an immunoglobulin-like molecule (e.g., a diagnostic reagent) that immunospecifically binds to full-length CEA protein but does not immunospecifically bind to short form CEA protein.

In certain embodiments, detecting a concentration of full-length CEA protein comprises contacting a sample with an antibody, an antigen binding fragment or an immunoglobulin-like molecule (e.g., a first diagnostic reagent) that immunospecifically binds to full-length CEA protein but does not immunospecifically bind to short form CEA protein, and detecting a concentration of short form CEA protein comprises contacting a sample with an antibody, an antigen binding fragment or an immunoglobulin-like molecule (e.g., a second diagnostic reagent) that immunospecifically binds to short form CEA protein but does not immunospecifically bind to full-length CEA protein.

In certain embodiments, the diagnostic reagent that immunospecifically binds to full-length CEA protein does not immunospecifically bind to other CEACAM family members and/or the diagnostic reagent that immunospecifically binds to short form CEA protein does not immunospecifically bind to other CEACAM family members.

In certain embodiments, the diagnostic reagent is an antibody, but with the proviso that the antibody is not A5B7 (the mouse monoclonal antibody known as A5B7). In certain embodiments, the diagnostic reagent is an antibody, but with the proviso that the antibody is not MEDI-565. In certain embodiments, the diagnostic reagent is an antibody, but with the proviso that the antibody is not the mouse monoclonal antibody known as IMMU-4 or arcitumomab. In certain embodiments, the diagnostic reagent is not a bispecific single chain antibody that includes an anti-CD3 binding portion. In certain embodiments, the diagnostic reagent is an antibody, but with the proviso that the antibody is not CEA.66, T84.66, or T84.12, as identified in Hefta et al. (1998) Immunotechnology 4: 49-57. In certain embodiments, the diagnostic reagent is an antibody, but with the proviso that the antibody is not PR1A3, as identified in Durbin (1994) PNAS 91: 4313. In certain embodiments, the diagnostic reagent is an antibody, but with the proviso that the antibody is not labetuzumab, as identified in Liersch (2005) JCO 23: 6763. In certain embodiments, the diagnostic reagent is an antibody, but with the proviso that the antibody is not the antibody provided with the CanAg CEA EIA kit, as distributed by Immuno Biological Laboratories Inc. using Vendor cat #401-85 (http://www.fdi.com/world_home/products/manual_kits/eia_kits/canag_cea.html).

In certain embodiments, the method may comprise a diagnostic step of detecting in said sample a concentration of an RNA encoding full-length and/or short form CEA protein. Said method may employ quantitative RT-PCR, primers and/or probes specific for full-length and/or short form CEA RNA, such as in a TaqMan assay, and may employ SAGE, MPSS, array-based methods, or direct sequencing.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In another aspect, the disclosure provides a method of detecting expression of short form carcinoembryonic antigen (CEA) RNA in a biological sample. The method comprises providing one or both of a nucleic acid probe or nucleic acid primers that hybridize to a CEA nucleotide sequence, and which probes and/or primers specifically identify expression of short form CEA by (i) hybridizing specifically to a short form CEA nucleotide sequence but not to a full-length CEA nucleotide sequence or (ii) hybridizing specifically to both short form CEA nucleotide sequence and full-length CEA nucleotide sequence in a manner that distinguishes expression of short form CEA from expression of full-length CEA. The method may include providing RNA from a biological sample. The method includes detecting expression of short form CEA RNA in said biological sample using said nucleic acid probe or nucleic acid primers.

In certain embodiments, the biological sample is a tumor tissue sample.

In certain embodiments, detecting expression comprises quantitative PCR or RT-PCR analysis. In certain embodiments, detecting expression comprises in situ hybridization analysis. In certain embodiments, the in situ hybridization analysis comprises FISH (fluorescent in situ hybridization). In certain embodiments, detecting expression comprises RNase protection analysis or Northern blot analysis. In certain embodiments, detecting expression comprises detecting expression with a microarray, SAGE, or MPSS.

In another aspect, the disclosure provides a method of detecting expression of short form carcinoembryonic antigen (CEA) protein in a biological sample. The method comprises providing an antibody, antigen binding fragment or immunoglobulin-like molecule (e.g., a diagnostic reagent) that immunospecifically binds to short form CEA protein but does not immunospecifically bind to full-length CEA protein. The method may include providing a biological sample. The method includes detecting expression of short form CEA protein in said biological sample using said antibody.

In certain embodiments, obtaining a sample comprises obtaining a blood sample, a urine sample, a fecal sample, or a sputum sample. In certain embodiments, obtaining a sample comprises obtaining a tumor biopsy or other tumor tissue sample. In certain embodiments, the sample is selected from one or more of whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites.

In certain embodiments, the antibody is a monoclonal antibody. In certain embodiments, the monoclonal antibody is a chimeric antibody, a humanized antibody, or a fully human antibody. In certain embodiments, the antibody is a polyclonal antibody. In certain embodiments, the diagnostic reagent binds to a protein comprising the amino acid sequence of SEQ ID NO: 1.

In certain embodiments, detecting expression comprises immunohistochemistry or immunocytochemistry analysis. In certain embodiments, detecting expression comprises ELISA analysis.

In another aspect, the disclosure provides a method of identifying patients that may be susceptible to a cancer therapeutic that immunospecifically binds to a target carcinoembryonic antigen (CEA) protein. In certain embodiments, the method comprises obtaining a sample from a patient, such as blood or tumor sample, for example a tumor biopsy, and detecting in the tumor sample expression of a target CEA RNA. In one example, the target CEA RNA is detected using a method such as Serial Analysis of Gene Expression (SAGE). In another example, the target CEA RNA is detected using Massively Parallel Signature Sequencing (MPSS). In another example, the target CEA RNA is detected using microarray that can specifically detect long and short form of CEA, such as an oligonucleotide array or an Affymetrix array. In another example, the target CEA RNA is detected using a probe and/or primers that distinguish RNA expression of full-length CEA from RNA expression of short form CEA. In another example, the CEA RNA may be reverse transcribed and sequenced, either in the form of a cDNA, or after cloning the cDNA in a suitable vector.

If the tumor sample from the patient expresses said target CEA RNA, the patient may be susceptible to treatment with a cancer therapeutic that immunospecifically binds to that target CEA protein. However, if the tumor sample from the patient does not express said target CEA RNA, the patient will not be susceptible to treatment with a cancer therapeutic that immunospecifically binds to that target CEA protein.

In certain embodiments, detecting target CEA RNA expression comprises contacting the sample with a probe and/or primers to evaluate expression of full-length CEA RNA. In certain embodiments, detecting target CEA RNA expression comprises contacting the sample with a probe and/or primers to evaluate expression of short form CEA RNA. In certain embodiments, detecting target CEA RNA expression comprises contacting the sample with one or more sets of probes and/or primers to evaluate expression of both full-length CEA RNA and short form CEA RNA.

In certain embodiments, the method further comprises obtaining from the patient one or more additional biological samples, and assaying the one or more biological samples for expression of the target CEA protein. For example, these additional one or more biological samples may be contacted with an antibody that immunospecifically binds to full-length CEA protein.

In certain embodiments, the method further comprises treating said subject with a cancer therapeutic.

In another aspect, the disclosure provides a method of generating antibodies immunospecific for full-length carcinoembryonic antigen (CEA) protein. The method comprises providing a portion of full-length CEA protein that is not present in short form CEA protein and using said portion of full-length CEA protein as an antigen for generating said antibodies.

In certain embodiments, the antibodies immunospecifically bind to full-length CEA protein but do not immunospecifically bind to short form CEA protein, and also do not immunospecifically bind to other related CEACAM protein family members (such as CEACAM 1, 3, 4, 6, 7, and 8).

In certain embodiments, said antibodies are monoclonal antibodies.

In certain embodiments, said antibodies are polyclonal antibodies.

In certain embodiments, the method includes generating an antigen binding fragment from said antibodies.

In certain embodiments, the method involves immunizing a non-human animal with a portion of full-length CEA protein that is not present in short form CEA protein.

In another aspect, the disclosure provides a method of generating antibodies immunospecific for short form CEA protein. In certain embodiments, the method comprises providing a fragment comprising a portion of consecutive amino acid residues present in short form CEA protein that are not present in full-length CEA protein and using said fragment as an antigen for generating said antibodies. An exemplary fragment includes consecutive amino acid residues that bridge the splice junction unique to short form CEA protein.

In certain embodiments, the fragment comprises the following consecutive amino acid residues: NIIQNELSVD (SEQ ID NO: 11). In certain embodiments, the fragment comprises the following consecutive amino acid residues: NIIQNKLSVD (SEQ ID NO: 12). In either case, the fragment may also include additional amino acid sequences such that the total size of the fragment is at least 10, 12, 14, 15, 16, 18, 20, 25, 30, 35, 40, 45, 50, 55, 60, 65, 70, 75, 80, 85, 90, 95, or 100 consecutive amino acids.

In certain embodiments, the fragment comprises substantially the same epitope as the foregoing fragments. A fragment comprising substantially the same epitope includes fragments of the foregoing with a small number (e.g., 1, 2, 3) of conservative amino acid substitutions.

In certain embodiments, the antibodies immunospecifically bind to short form CEA protein but do not immunospecifically bind to full-length CEA protein, and also do not immunospecifically bind to other related CEACAM protein family members (such as CEACAM 1, 3, 4, 6, 7, and 8).

In certain embodiments, the antibodies are immunospecific for a short form CEA polymorphism comprising NIIQNELSVD (SEQ ID NO: 11), and are not immunospecific for a short form CEA polymorphism comprising NIIQNKLSVD (SEQ ID NO: 12). In other embodiments, the antibodies are immunospecific for a short form CEA polymorphism comprising NIIQNKLSVD (SEQ ID NO: 12), and are not immunospecific for a short form CEA polymorphism comprising NIIQNELSVD (SEQ ID NO: 11). In other embodiments, the antibodies are immunospecific for both of the foregoing short form CEA polymorphisms.

In certain embodiments, said antibodies are monoclonal antibodies.

In certain embodiments, the method includes generating an antigen binding fragment from said antibodies.

In certain embodiments, the method involves immunizing a non-human animal with a portion of short form CEA protein that is not present in full-length CEA protein.

In another aspect, the disclosure provides a purified polypeptide comprising the amino acid sequence represented in SEQ ID NO: 1 (in the presence or absence of pro-sequences), or a fragment thereof comprising the following consecutive amino acid residues: NIIQNELSVD (SEQ ID NO: 11). In certain embodiments, the disclosure provides a purified polypeptide comprising the amino acid sequence represented in SEQ ID NO: 1 (in the presence or absence of all or a portion of N and/or C terminal pro-sequences), or a fragment thereof comprising the following consecutive amino acid residues: NIIQNKLSVD (SEQ ID NO: 12). The N-terminal pro sequence is an approximately 34 amino acid residue signal peptide and the C-terminal pro sequence is an approximately 17 amino acid peptide.

In certain embodiments, the purified polypeptide comprises the amino acid sequence represented in SEQ ID NO: 1 (in the presence or absence of pro-sequences absent from mature CEA). In certain embodiments, the purified polypeptide comprises a fragment of SEQ ID NO: 1 comprising the following consecutive amino acid residues: NIIQNELSVD (SEQ ID NO: 11). In certain embodiments, the purified polypeptide comprises a fragment of SEQ ID NO: 1 comprising the following consecutive amino acid residues: NIIQNKLSVD (SEQ ID NO: 12). In certain embodiments, said fragment is approximately 10 consecutive amino acid residues. In certain embodiments, said fragment is approximately 12, 14, 15, 16, 18, or 20 consecutive amino acids. In certain embodiments, said fragment is approximately 25, 30, 33, 35, 40, 45, 48, 50, 55, 60, 65, 70, 75, 80, 85, 90, 95, or 100 consecutive amino acid residues. In certain embodiments, said fragment is at least 20, at least 25, at least 30, at least 35, at least 40, at least 45, or at least 50 consecutive amino acid residues. In certain embodiments, said fragment is less than 250, less than 200, less than 175, less than 150, less than 125, less than 100, less than 90, less than 85, less than 80, less than 75, less than 70, less than 65, less than 60, less than 55, or less than 50 consecutive amino acid residues. In certain embodiments, said fragment is at least 100, at least 150, at least 200, at least 250, at least 300, at least 350, or at least 400 consecutive amino acid residues.

In certain embodiments, the purified polypeptide is conjugated to an adjuvant.

In certain embodiments, the purified polypeptide is used to generate immunospecific antibodies.

In certain embodiments, a nucleotide sequence encoding the purified polypeptide is used to generate immunospecific antibodies.

In another aspect, the disclosure provides diagnostic methods having any of the properties described herein, but wherein the diagnostic reagent is administered to a patient. Following administration to a patient, CEA expression can be visualized using in vivo imaging techniques. Alternatively, a sample can be taken from the patient and CEA concentration can be assessed ex vivo (e.g., concentration is assayed ex vivo but the contact between the diagnostic reagent and CEA protein occurs in vivo). This aspect of the disclosure can be applied to and combined with any one or more of the aspects and embodiments of the disclosure described in detail herein.

In certain embodiments of any of the foregoing or following aspects or embodiments, the method may comprise treating the subject, such as the human patient, with an anti-CEA cancer therapeutic. In certain embodiments, such anti-CEA cancer therapeutic immunospecifically binds to the same or substantially the same epitope as that immunospecifically bound by the diagnostic reagent used in the detecting steps. In certain embodiments, the anti-CEA cancer therapeutic is a bispecific antibody (including a bispecific single chain antibody) including an anti-CEA portion and an anti-CD3 portion. In certain embodiments, the anti-CEA cancer therapeutic is the bispecific antibody MEDI-565. In certain embodiments, the anti-CEA cancer therapeutic is a bispecific antibody having an anti-CEA portion that is the same as MEDI-565 or an anti-CEA portion that binds the same or substantially the same epitope as MEDI-565. In certain embodiments, the anti-CEA cancer therapeutic includes an anti-CEA portion that is the same as MEDI-565 or an anti-CEA portion that binds the same or substantially the same epitope as MEDI-565, but which anti-CEA therapeutic is not a bispecific antibody. In certain embodiments, the therapeutic regimen comprises treatment with a bispecific antibody (including a bispecific single chain antibody) that includes both an anti-CEA portion and an anti-CD3 portion.

In certain embodiments, the therapeutic to be used includes, at least, a CEA binding portion comprising the amino acid sequence represented in any of SEQ ID NOs: 28-44 and 46-51. In certain embodiments, the therapeutic to be used is a bispecific antibody comprising the amino acid sequence represented in any of SEQ ID NOs: 28-44 and 47. In certain embodiments, the therapeutic to be used is a bispecific antibody comprising the amino acid sequence represented in any of SEQ ID NOs: 34, 36, 41, 42, 43, and 47. In certain embodiments, the therapeutic to be used is a bispecific antibody comprising the amino acid sequence represented in any of SEQ ID NOs: 37-40. In certain embodiments, the therapeutic to be used is a bispecific antibody comprising the amino acid sequence represented in SEQ ID NO: 48. In certain embodiments, the therapeutic to be used is a bispecific antibody comprising the amino acid sequence represented in SEQ ID NO: 49. In certain embodiments, the therapeutic to be used is a bispecific antibody comprising the amino acid sequence represented in SEQ ID NOs: 48 and 49. In certain embodiments, the therapeutic to be used is a bispecific antibody comprising the amino acid sequence represented in SEQ ID NO: 46. In any of the forgoing or previous embodiments, SEQ ID NO: 46 may father comprise six histidines on the C-terminus.

In certain embodiments of any of the foregoing or following aspects or embodiments, the cancer therapeutic immunospecifically binds to an epitope on CEA protein that is the same or substantially the same as that immunospecifically bound by said antibody, antigen binding fragment or immunoglobulin-like molecule used in said detecting steps (e.g., the diagnostic reagent).

In certain embodiments of any of the foregoing or following aspects or embodiments, the method comprises more than one diagnostic step, and the same diagnostic reagent is used at each diagnostic step. In certain embodiments, the method comprises more than one diagnostic step, and although the same diagnostic reagent is not used at all steps, each of the diagnostic reagents bind the same or substantially the same epitope.

In certain embodiments of any of the foregoing or following aspects or embodiments, the subject is a human.

In certain embodiments of any of the foregoing or following aspects or embodiments, the diagnostic and/or therapeutic reagent immunospecifically binds to human CEA.

In certain embodiments of any of the foregoing or following aspects or embodiments, the diagnostic step or steps are performed ex vivo (e.g., outside of the patient's body).

In certain embodiments of any of the foregoing or following aspects or embodiments, the method includes one or more treatment steps. Exemplary treatments include one or more of surgery, chemotherapy, radiation therapy, immunotherapy, biological therapy, herbal therapy, acupuncture, or an anti-CEA cancer therapeutic. A suitable treatment regimen includes any one or more of these and other treatment modalities delivered according to a dosage and time course prescribed by a suitable medical professional.

In certain embodiments of any of the foregoing or following aspects or embodiments, the cancer therapeutic comprises a protein therapeutic. In certain embodiments, the protein therapeutic is an antibody or antigen binding fragment. In certain embodiments, the antibody or antigen binding fragment is from a monoclonal antibody. In certain embodiments, the monoclonal antibody is a chimeric antibody, a humanized antibody, or a fully human antibody. In certain embodiments, the protein therapeutic immunospecifically binds to a protein comprising the amino acid sequence of SEQ ID NO: 2 (mature full-length human CEA). In certain embodiments, the protein therapeutic immunospecifically binds to a protein comprising the amino acid sequence of SEQ ID NO:2, but which protein therapeutic does not immunospecifically bind to a protein comprising the amino acid sequence of SEQ ID NO: 1 in the presence and/or absence of the pro-sequence. In certain embodiments, the protein therapeutic immunospecifically binds to a protein comprising the amino acid sequence of SEQ ID NO:2, but which protein therapeutic does not immunospecifically bind to a protein comprising the amino acid sequence of SEQ ID NO: 1 (in the presence or absence of pro-sequences) and does not immunospecifically bind to other CEACAM family members. In certain embodiments, the protein therapeutic comprises an antigen binding domain of antibody A5B7. In certain embodiments, the protein therapeutic is a bispecific antibody. In certain embodiments, the bispecific antibody is MEDI-565.

In certain embodiments of any of the foregoing or following aspects or embodiments, the sample (e.g., the biological sample on which diagnostic testing is performed) is selected from one or more of: whole blood, serum, plasma, saliva, urine, feces, seminal plasma, sweat, amniotic fluid, sputum, breast milk, breast nipple aspirates or other fluid including cells from the breast, bile, tissue homogenate, pleural effusion fluids, and ascites. Note that such biological samples may contain both cellular and non-cellular elements. In certain embodiments, the sample is a tumor tissue sample. For embodiments in which multiple samples are taken, the disclosure contemplates that each of those samples may be from the same tissue source (e.g., serum or feces), or the samples may be from different tissue sources. Furthermore, the disclosure contemplates that a diagnostic step may include detecting CEA expression or concentration in a sample from a single source or may include detecting CEA expression or concentration in samples from more than one tissue source.

In certain embodiments of any of the foregoing or following aspects or embodiments, the CEA-expressing cancer may be any one or more of the following: colon cancer, rectal cancer, pancreatic cancer, esophageal cancer, gastroesophageal cancer, stomach cancer, lung cancer and breast cancer. In certain embodiments, the CEA-expressing cancer is colon cancer. In certain embodiments, the classification of the type of cancer (e.g., pancreatic or colon) refers to the classification of the initial tumor—although it is recognized that metastases may appear in other tissues. In such cases, the cancer will still be categorized as, for example, a CEA-expressing colon cancer, even though the cancer may have metastasized to non-colon tissue. Any of the cancers discussed herein may be primary or metastatic (e.g., metastatic colorectal cancer).

In certain embodiments of any of the foregoing or following aspects or embodiments, the detecting step comprises contacting the sample with said antibody, antigen binding fragment or immunoglobulin-like molecule and detecting the concentration of full-length CEA protein by immunohistochemistry or immunocytochemistry. In certain embodiments of any of the foregoing or following aspects or embodiments, the detecting step comprises contacting the sample with said antibody, antigen binding fragment or immunoglobulin-like molecule and detecting the concentration of full-length CEA protein by ELISA.

In certain embodiments of any of the foregoing or following aspects or embodiments, the diagnostic reagent is a monoclonal antibody. In certain embodiments, the monoclonal antibody is a chimeric antibody, a humanized antibody, or a fully human antibody. In some embodiments, the antibody is a polyclonal antibody.

In certain embodiments of any of the foregoing or following aspects or embodiments, the detecting step comprises contacting the sample with the diagnostic reagent (said antibody, antigen binding fragment or immunoglobulin-like molecule), which antibody, antigen binding fragment or immunoglobulin-like molecule immunospecifically binds to a protein comprising the amino acid sequence of SEQ ID NO:2 and detecting the concentration of full-length CEA protein by immunohistochemistry or ELISA.

In certain embodiments of any of the foregoing or following aspects or embodiments, the detecting step comprises contacting the sample with an antibody or antigen binding fragment comprising the antigen binding domain of antibody A5B7, with the proviso that the antibody is not A5B7.

In certain embodiments of any of the foregoing or following aspects or embodiments, the detecting step comprises contacting said sample with an antibody or antigen binding fragment comprising an antigen binding domain that binds the same or substantially the same epitope as A5B7, with the proviso that the antibody is not A5B7.

In certain embodiments of any of the foregoing or following aspects or embodiments, the detecting step comprises contacting said sample with an antibody or antigen binding fragment comprising an antigen binding domain that binds the same or substantially the same epitope as A5B7, with the proviso that the antibody is not A5B7 or MEDI-565.

In certain embodiments of any of the foregoing or following aspects or embodiments, the cancer therapeutic comprises a bispecific antibody (including a bispecific single chain antibody) that includes an anti-CEA portion and an anti-CD3 portion. In certain embodiments of any of the foregoing or following aspects or embodiments, the cancer therapeutic includes MEDI-565. In certain embodiments of any of the foregoing or following aspects or embodiments, the diagnostic reagents comprises a bispecific antibody (including a bispecific single chain antibody) that includes an anti-CEA portion and an anti-CD3 portion. In certain embodiments of any of the foregoing or following aspects or embodiments, the diagnostic reagent is MEDI-565. In certain embodiments of any of the foregoing or following aspects or embodiments, both the cancer therapeutic and the diagnostic reagent comprise a bispecific antibody (including a bispecific single chain antibody) that includes an anti-CEA portion and an anti-CD3 portion. In certain embodiments of any of the foregoing or following aspects or embodiments, both the cancer therapeutic and the diagnostic reagent include MEDI-565.

In certain embodiments of any of the foregoing or following aspects or embodiments, the diagnostic reagent is selected from A5B7 (the mouse monoclonal antibody known as A5B7), arcitumomab, CEA.66, T84.66, T84.12, PR1A3, or labetuzumab.

In certain embodiments of any of the foregoing or following aspects or embodiments, the cancer therapeutic includes MEDI-565 or an antibody comprising an anti-CEA portion that is the same or that binds the same or substantially the same epitope as MEDI-565. In certain embodiments of any of the foregoing or following aspects or embodiments, the diagnostic reagent is MEDI-565 or an antibody comprising an anti-CEA portion that is the same or that binds the same or substantially the same epitope as MEDI-565. In certain embodiments of any of the foregoing or following aspects or embodiments, both the cancer therapeutic and the diagnostic reagent include MEDI-565, or comprise an anti-CEA portion that is the same or that binds the same or substantially the same epitope as MEDI-565.

In certain embodiments, the diagnostic reagent (e.g., the antibody, antigen binding fragment or immunoglobulin-like molecule) immunospecifically binds to full-length CEA protein but does not immunospecifically bind to short form CEA protein. In some embodiments, the diagnostic reagent also does not immunospecifically bind to other related CEACAM protein family members (such as CEACAM 1, 3, 4, 6, 7, and 8). Thus, in certain embodiments, the diagnostic reagent is not a pan-CEA family member antibody, but is specific for full-length CEA protein.

Specificity with respect to a diagnostic or therapeutic reagent that immunospecifically binds to full-length (or short form) CEA is intended to refer to immunospecificity for mature CEA (without regard for the presence or absence of pro-sequences). However, it is readily appreciated that an antibody immunospecific for mature full-length CEA protein may also immunospecifically bind to full-length CEA in the presence of pro-sequences. Similarly, an antibody immunospecific for short form CEA protein may also immunospecifically bind to short form CEA in the presence (or absence of pro-sequences). An antibody will be considered immunospecific for a target CEA protein as long as it immunospecifically binds to the mature target CEA protein (and does not immunospecifically bind to non-target CEA proteins). However, the pro-protein corresponding to that mature target CEA will not be considered another form or a non-target form of CEA. Similarly, binding to both soluble and membrane anchored mature target CEA will not be considered binding to a non-target form of CEA.

In certain embodiments of any of the foregoing or following, CEA concentration is compared to a standard range reflecting full-length CEA protein concentration in samples from healthy subject. In certain embodiments, the standard range is less than or equal to 3 ug/L (3 ng/mL) in serum of non-smokers and less than or equal to 5 ug/L (5 ng/mL) in serum of smokers. In certain embodiments, the standard range is less than or equal to 5 ug/L (5 ng/mL) in serum, regardless of smoking status.

The disclosure contemplates all combinations of any of the foregoing aspects and embodiments, as well as combinations with any of the embodiments set forth in the detailed description and examples.

### BRIEF DESCRIPTION OF THE DRAWINGS

FIG. 1A provides a schematic representation of the exon structure of full-length CEA and short form CEA. The boxes represent exons, and the exons are numbered. The splice variant short form CEA is missing exons 3 and 4, and part of exon 2, relative to full-length CEA. FIG. 1B provides a representation of the domain structure of the full-length and short form CEA. IgV-like refers to Immunoglobulin Variable like domain; Ig refers to the various Immunoglobulin-like domains. The lines in these schematics indicate GPI linkage of the proteins to the plasma membrane. The N-terminus of each protein begins on the left side of each schematic.

FIG. 2 provides an alignment of the amino acid sequence for full-length CEA and short form CEA (CEA splice variant). In FIG. 2, the CEA proteins are shown with the N- and C-terminal pro-sequences indicated in black boxes. These pro-sequences are not typically present in the mature CEA protein. The amino acid sequence for short form CEA (CEA splice variant) is provided as SEQ ID NO: 1. The amino acid sequence for mature full-length CEA, without N- and C-terminal pro-sequences, is provided as SEQ ID NO: 2. Note that the regions of the protein identified in the examples as important for antibody binding are also boxed and labeled as “epitope residues 326-349” and “epitope residues 388-410”. In FIG. 2, these regions are indicated using numbering that includes the N-terminal signal sequence. Thus, these regions can also be referred to using numbering relative to the mature CEA protein as “epitope residues 292-315” and “epitope residues 354-376.”

FIG. 3 provides a comparison of the domain structure of full-length CEA versus that of short form CEA. In FIG. 3, Ig refers to immunoglobulin-like domains, V-set refers to Ig variable region-like N-terminal domain, and numbers represent sequential V-set and Ig domains of mature proteins. Note that these Ig domains are also referred to as IgC domains, and are numbered sequentially from 1 through 6. The schematic provided in FIG. 3 is an alternative illustration of the schematic provided in FIG. 1B.

FIG. 4 provides the results of a screen of single cell clones of CHO cells for expression of short form CEA. CHO cells infected with lentivirus carrying the sequence for short form CEA were screened for cell surface expression of short form CEA. Experiments were performed with IgG B9 and with anti-CEACAM5 monoclonal antibody using a FACS-based approach. <FITC-A> denotes the relative fluorescence intensity of bound antibody. The 72 series numbers along the right hand side of the figure refer to cell line clone numbers. Parental CHO cells lacking expression of CEA are referred to as CHO dhfr-. CHO cells expressing full-length human CEA are referred to as CHOhuCEA.

FIG. 5 depicts the ability of various anti-CEA antibodies to bind to single cell CHO cell clones 72-4 and 72-14, which express short form CEA. FITC-A denotes the relative fluorescence intensity of bound antibody. 2° only refers to anti-mouse IgG Alexa Fluor 488 conjugated secondary antibody bound in the absence of primary antibody. Panel A shows the lack of expression of CEA (full-length or short form) observed in parental dhfr-CHO cells lacking expression of CEA. Panel B shows that the B9 IgG and CEACAM5 antibody, as well as the pan-antibodies CEACAM 1, 3, 4, 5, 6 and CEACAM 1, 3, 5, 6 bind CHO cells expressing full-length CEA. Panel C shows that the CEACAM5 antibody also binds to CHO cell clone 72-4, which expresses short form CEA. However, B9 IgG, and the pan-antibodies CEACAM 1, 3, 4, 5, 6 and CEACAM 1, 3, 5, 6 do not appear to significantly bind to clone 72-4. Panel D shows that the CEACAM5 antibody also binds to CHO cell clone 72-14, which expresses short form CEA. However, B9 IgG, and the pan-antibodies CEACAM 1, 3, 4, 5, 6 and CEACAM 1, 3, 5, 6 do not appear to significantly bind to clone 72-14.

FIG. 6 shows the results of experiments examining T cell mediated killing by MEDI-565 of CHO cells expressing short form CEA. CHO cells expressing full-length CEA (CHOhuCEA; circle), parental CHO cells lacking CEA expression (CHO dhfr-; square), and CHO cell short form CEA expressing clones 72-4 and 72-14 (CHO-CEA SV 72-4; triangle and CHO-CEA SV 72-14; upside down triangle) were tested for susceptibility to killing by CD3+ enriched T cells from two individuals (donors 1 and 2) in the presence of MEDI-565. Panel A depicts the results from experiments using material from donor 1 and shows that only cells expressing full-length CEA are susceptible to T cell mediated killing in the presence of MEDI-565. Panel B depicts similar results obtained using material from donor 2.

FIG. 7 shows the results of experiments examining activation of human T cells by MEDI-565 in the presence of CHO cell clones that express short form CEA. Activation of CD8+ or CD4+ T cells is assessed by increased surface CD25 levels. CD25-PE-MFI refers to mean fluoresence intensity of bound PE-labeled mouse anti-human CD25 antibody. CD4+ and CD8+ T cells from donor 1 (panel A) or donor 2 (panel B) were incubated with various doses of MEDI-565 and either (i) CHO cells expressing full-length CEA (CHOhuCEA; circle), (ii) parental CHO cells lacking CEA expression (CHO dhfr-; square), (iii) CHO cell short form CEA expressing clone 72-4 (CHO-CEA SV 72-4; triangle), or (iv) CHO cell short form CEA expressing clone 72-14 (CHO-CEA SV 72-14; upside down triangle). Panel A depicts the results of experiments using material from donor 1 and shows that MEDI-565 activates CD8+ T cells and CD4+ T cells only in the presence of CHO cells expressing full-length CEA. Panel B depicts similar results obtained using material from donor 2.

FIG. 8 shows the results of experiments examining deletion mutants of human CEA. Deletion mutants were constructed by deleting indicated IgC-like domains (panel A). Expression of deletion mutants and wild type CEA was monitored using an anti-CEA polyclonal antibody (“poly”; upper row, panel B). Transfectants were incubated with 10 μg/mL of MEDI-565, followed by anti-penta-His Alexa Fluor 488 (“penta-His” disclosed as SEQ ID NO: 27), and detected by flow cytometry (lower row, panel B).

FIG. 9 shows the results of experiments examining swap mutants between IgC3 and IgC5 domains of CEA. Sequence homology analysis revealed 21 residues different between IgC3 and IgC5 domains of CEA (panel A). Three segments were defined as A, B, and C in IgC3 and IgC5 domains (panel A) to generate swap mutants. Swap mutants were constructed by exchanging segments A, B, or C between IgC3 and IgC5 using truncated protein IgC3_GPI or IgC5_GPI as templates, which encode the N-domain, IgC3 or IgC5 domain, and GPI region (panel B). Expression of swap mutants was monitored using an anti-CEA polyclonal antibody (“poly”; upper row, panel C). Transfectants were incubated with 10 μg/mL of MEDI-565, followed by penta-His Alexa Fluor 488 (“penta-His” disclosed as SEQ ID NO: 27), and detected by flow cytometry (lower row, panel C). Substitution of segments A (KO_A) or C (KO_C) of IgC3 with the counterpart of IgC5 abolished the binding of MEDI-565. Grafting both segments A and C of IgC3 into IgC5 led to a gain in binding to MEDI-565.

FIG. 10 shows the results of experiments examining critical residues of CEA involved in the binding of MEDI-565. The residues that differ between IgC3 and IgC5 in segment A and C of IgC3 were replaced with the corresponding residues of IgC5: F²⁹²T²⁹⁴N²⁹⁹(KO_FTN), N²⁹⁹E³⁰⁴(KO_NE), E³⁰⁴L³⁰⁹I³¹⁵ (KO_ELI), V³⁵⁴, G³⁵⁵P³⁵⁶E³⁵⁸(KO_VGPE), E³⁶⁴L³⁶⁵V³⁶⁷D³⁶⁸H³⁶⁹(KO_ELVDH), and I³⁷⁴N³⁷⁶(KO_IN) (panel A). Mutants KO_FTN and KO_NE showed no binding to MEDI-565 (panel A). A modeled structure of the IgC3 domain of CEA was constructed using the crystal structure of murine CEACAM1A (33.7% sequence homology) as a template with SWISS-MODEL workspace (panel B). The modeled structure showed that two clusters of residues (V³⁵⁴G³⁵⁵P³⁵⁶E³⁵⁸ and I³⁷⁴N³⁷⁶) in segment C were spatially close to the critical residue N²⁹⁹ of segment A. Based on the results in panel A and the modeled structure, three additional mutants were constructed, replacing N²⁹⁹ of IgC3 with the corresponding residue of IgC5 (KO_N), substituting V³⁵⁴G³⁵⁵P³⁵⁶E³⁵⁸ and I³⁷⁴N³⁷⁶ of IgC3 with the counterparts of IgC5 (KO_VGPE+IN), and grafting F²⁹²T²⁹⁴N²⁹⁹, V³⁵⁴G³⁵⁵P³⁵⁶E³⁵⁸, and I³⁷⁴N³⁷⁶ of IgC3 into IgC5 (KI_FTN+VGPE+IN) (panel C). Knocking-out the residue N²⁹⁹ abolished MEDI-565 binding. Grafting F²⁹²T²⁹⁴N²⁹⁹, V³⁵⁴G³⁵⁵P³⁵⁶E³⁵⁸, and I³⁷⁴N³⁷⁶ into IgC5 led to gain in binding of MEDI565. Amino acid numbering is based on the mature CEA protein.

FIG. 11 shows the results of experiments examining the specificity of MEDI-565 for full-length CEA over the short-form splice variant. Panel A depicts expression of full-length CEA and CEA splice variant proteins in CHO cells as determined by flow cytometry. MEDI-565 (10 μg/mL) and the CEACAM5-specific mAb (clone 26/3/13; 10 μg/mL) were used to detect by flow cytometry expression levels of full-length CEA and co-expression of full-length CEA and CEA splice variant expression in CHO cells, respectively. Mouse IgG1, mouse IgG1 control antibody. <FITC-A>, fluorescence in FITC channel. MEDI-565 induced human T-cell killing is depicted in panel B. Activation of human CD8+ T cells and CD4+ T cells are depicted in panels C and D, respectively. For panel B, CHO DHFR−, CHO FL, CHO SV and CHO FL+SV cells were tested for their susceptibility to be killed by CD3+ T cells from 3 individual donors in the presence of MEDI-565 at the indicated concentrations. Experiments were performed in duplicate. EC₅₀ values listed indicate the mean value among 3 donors ±SEM. p=0.79 comparing cytotoxicity EC₅₀ values between CHO FL CEA and CHO FL+SV CEA cells. The activation (increased surface CD25 levels) of CD8+ (panel C) and CD4+ (panel D) T cells isolated from each of the 3 healthy donors was investigated concurrently with the cytotoxicity assays shown in panel B. p=0.60 comparing CD8+ T cell activation EC₅₀ values between CHO FL CEA and CHO FL+SV CEA; p=0.15 comparing CD4+ T cell activation EC₅₀ values between CHO FL CEA and CHO FL+SV CEA. MFI CD25-APC=median fluorescence intensity of bound APC labeled, anti-human CD25 mAb. Experiment was repeated once with similar results.

### DETAILED DESCRIPTION OF THE DISCLOSURE

(i) Introduction

Carcinoembryonic antigen (CEA; CEACAM5; CD66e) is a glycosylated human oncofetal antigen that belongs to the CEA-related cell adhesion molecule (CEACAM) family of the immunoglobulin gene superfamily. CEACAM5 is closely related to CEACAM1, CEACAM3, CEACAM4, CEACAM6, CEACAM7 and CEACAM8. CEA has been suggested to mediate cell-cell adhesion, facilitate bacterial colonization of the intestine, and protect the colon from microbial infection by binding and trapping infectious microorganisms. As used herein, CEA refers to CEACAM5, particularly human CEACAM5.

CEA is expressed at low levels in normal tissues of epithelial origin (Hammarstrom, 1999) in a polarized manner, and such expression is only observed at the luminal portion of the cell. In contrast, expression of CEA is high in carcinomas (including colon, pancreatic, gastric, esophageal, lung, breast, uterine, ovarian, and endometrial) and in a subset of melanomas (Hammarstrom, 1999; Sanders et al, 1994). Cancer cells not only lose polarized (luminal) expression of CEA, but actively cleave CEA from their surface by phospholipases, an action that results in high serum levels of CEA (Hammarstrom, 1999).

Serum levels of CEA serve as a useful prognostic indicator in patients with gastrointestinal cancers (Duffy, 2001, Locker et al, 2006; Rother, 2007); elevated levels indicate a poor prognosis and correlate with reduced overall survival. Additionally, there are therapies being developed to treat CEA-expressing cancers. Despite the development of diagnostic tools and putative therapeutics, much remains unknown about CEA.

Prior to the experiments described herein, it was assumed that CEA expressed on certain cancer cells and shed into serum, as well as other bodily fluids, was a single protein, albeit a protein observed bound to cell surfaces and in serum and other fluids. Thus, it was assumed that reagents that detected CEA expression were detecting expression of a single protein expressed on cell surfaces and found in certain fluids, and that compounds that targeted CEA to provide a therapeutic effect were binding to this protein. However, as detailed herein, CEA appears to exist in at least two forms: a full-length form, corresponding to an approximately 702 amino acid protein (prior to removal of an approximately 34 amino acid N-terminal signal peptide pro-sequence and an approximately 17 amino acid C-terminal pro-sequence not present in the mature protein) typically referred to as CEA, and a splice variant, referred to herein as short form CEA. The existence of two forms of CEA raises questions about the specificity and relevance of diagnostic tests based on detecting CEA in cells or fluids. For example, if a diagnostic test unwittingly relies on a reagent that can recognize both the full-length and the short form CEA, it is unclear whether the test will provide consistent results of biological relevance. As such, identification of short form CEA, and the appreciation that human CEA exists in both a full-length and short form (both of which may be detected on cell surfaces or in fluids), provides critical information that allows the making and testing of improved diagnostic and therapeutic reagents.

Additionally, as detailed herein, identification of short form CEA allows analysis of expression of short form CEA in samples from healthy patients and in CEA-expressing cancers and biological fluids. Further, identification of short form CEA allows, for example, analysis of: concordance/discordance of expression of full-length CEA versus short form CEA; prognostic value of short form CEA expression; prognostic value of the ratio of full-length CEA to short form CEA; and generation and/or optimization of specific diagnostic reagents that target either full-length or short form CEA, but not both. Moreover, appreciation of the existence of two forms of CEA, as well as the potential value of generating diagnostic and therapeutic reagents that are specific for only one such form, allows the use of matched reagents. In other words, health care providers can select for diagnostic purposes a reagent that recognizes the same or substantially the same epitope of CEA as that recognized by the therapeutic reagent that targets CEA as part of the patient's treatment plan. This type of matching ensures that the diagnostic and therapeutic reagents have the same specificity for a CEA form. Without being bound by theory, such matching likely improves the correlation between therapeutic efficacy and diagnostic data.

In certain aspects and embodiments, the methods of the disclosure utilize diagnostic reagents that bind to the same or substantially the same epitope as a therapeutic reagent (e.g., a cancer therapeutic). In this context, two reagents bind to substantially the same epitope if they can compete with one another, at relevant concentrations, for binding to that epitope. For example, reagents that bind to overlapping epitopes would be referred to as reagents that bind to substantially the same epitope. Such antibodies may said to be competitive inhibitors.

(ii) Terminology

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

As used herein, the term “immunoglobulin-like molecule” refers to an antibody mimic or antibody-like scaffold. In certain embodiments, immunoglobulin-like molecules may be any polypeptide comprising a non-immunoglobulin antigen binding scaffold, including, single chain antibodies, diabodies, minibodies, etc. Immunoglobulin-like molecules may contain an immunoglobulin-like fold. In certain aspects, the immunoglobulin-like molecules may be derived from a reference protein by having a mutated amino acid sequence. In certain embodiments, the immunoglobulin-like molecule may be derived from an antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

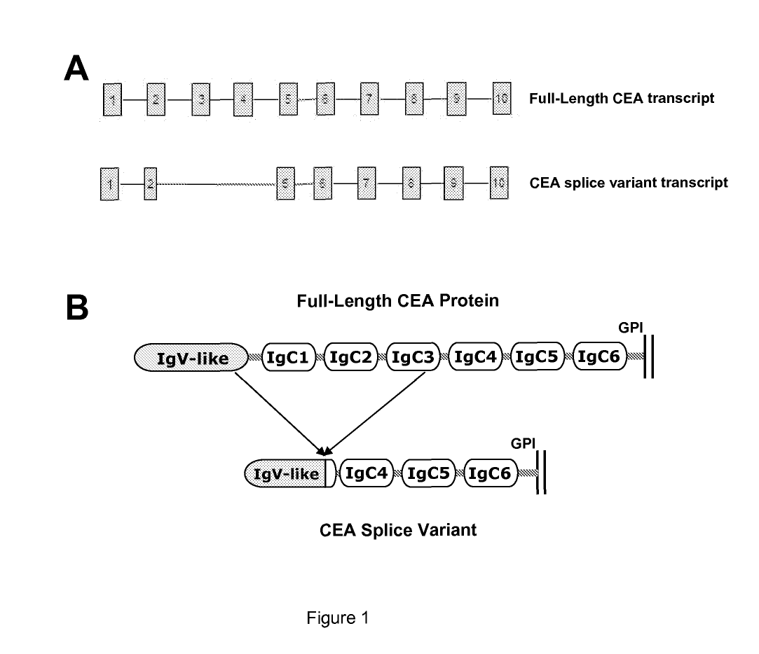

As used herein, the term “full-length CEA” refers to the full-length CEACAM5 protein coded by 10 exons or to a nucleotide sequence that encodes such a protein. The full-length CEA protein is approximately 702 amino acids (prior to removal of N- and C-terminal pro-sequences; N-terminal pro-sequence is approximately 34 amino acid signal sequence and C-terminal pro-sequence is approximately 17 amino acid sequence). The amino acid sequence for full-length CEA protein, including the pro-sequences, is set forth in FIG. 1 and can also be found in GenBank at NCBI RefSeq NP_{—}004354.2. Mature full-length CEA protein is set forth in SEQ ID NO: 2. Note that the sequence set forth in SEQ ID NO: 2 references a particular polymorphism (shown bolded and underlined). Other CEA polymorphisms have been identified in the art. However, the indicated polymorphism occurs at the splice junction unique to short form CEA, and thus has potential relevance to the instant disclosure. For example, it is possible that polymorphisms at this splice junction may influence binding specificity of particular antibodies. In certain embodiments, full-length CEA refers to the mature full-length form of CEA (without pro-sequences). Specificity with respect to a diagnostic or therapeutic reagent that immunospecifically binds to full-length (or short form) CEA is intended to refer to immunospecificity for mature CEA (without regard for the presence or absence of pro-sequences). However, it is readily appreciated that, for example, an antibody immunospecific for mature full-length CEA protein may also immunospecifically bind to full-length CEA in the presence of pro-sequences. A reagent will be considered immunospecific for a target CEA protein as long as it immunospecifically binds to the mature target CEA protein (and does not immunospecifically bind to non-target CEA proteins). However, the pro-protein corresponding to that mature target CEA will not be considered another form or a non-target form of CEA. Similarly, binding to both soluble and membrane anchored mature target CEA will not be considered binding to a non-target form of CEA, nor will it be considered as evidence of lack of immunospecificity. Further, although it is appreciated that certain reagents may be immunospecific for a particular CEA polymorphism, immunospecificity of a reagent for more than one polymorphism of a target CEA will not be considered binding to a non-target form of CEA, nor will it be considered as evidence of lack of immunospecificity.

As used herein, the term “short form CEA” refers to a splice variant of CEACAM5 of approximately 420 amino acids or a nucleotide sequence that encodes such a protein. The short form CEA protein is a splice variant of full-length CEA missing exons 3 and 4. In other words, the short form CEA includes an in-frame truncation from residues 116 to 396 (when numbered to include the N-terminal pro-sequence) of the full-length CEA. The amino acid sequence for short form CEA protein, including pro-sequences, is set forth in SEQ ID NO: 1. Note that the sequence set forth in SEQ ID NO: 1 references a particular polymorphism (shown bolded and underlined). Other CEA polymorphisms have been identified in the art. However, the indicated polymorphism occurs at the splice junction unique to short form CEA, and thus has potential relevance to the instant disclosure. In certain embodiments, short form CEA refers to the mature short form of CEA without pro-sequences not present in the mature protein. Specificity with respect to a diagnostic or therapeutic reagent that immunospecifically binds to short form CEA is intended to refer to immunospecificity for mature CEA (without regard for the presence or absence of pro-sequences). However, it is readily appreciated that, for example, an antibody immunospecific for mature short form CEA protein may also immunospecifically bind to short form CEA in the presence of pro-sequences. A reagent will be considered immunospecific for a target CEA protein as long as it immunospecifically binds to the mature target CEA protein (and does not immunospecifically bind to non-target CEA proteins). However, the pro-protein corresponding to that mature target CEA will not be considered another form or a non-target form of CEA. Similarly, binding to both soluble and membrane anchored mature target CEA will not be considered binding to a non-target form of CEA, nor will it be considered as evidence of lack of immunospecificity. Further, although it is appreciated that certain reagents may be immunospecific for a particular CEA polymorphism, immunospecificity of a reagent for more than one polymorphism of a target CEA will not be considered binding to a non-target form of CEA, nor will it be considered as evidence of lack of immunospecificity.

As used herein, the term “immunospecifically binds” refers to the situation in which one member of a specific binding pair, such as an antibody, does not significantly bind to molecules other than its specific binding partner(s) (i.e., cross-reactivity of less than about 25%, 20%, 15%, 10%, or 5%) as measured by a technique in the art, at a diagnostically or therapeutically relevant concentration e.g., by competition ELISA or by measurement of K_{D} with BIACORE or KINEXA assay. The term is also applicable where e.g., an antigen-binding domain of an antibody of the disclosure is specific for a particular epitope that is carried by a number of antigens, in which case the specific antibody carrying the antigen-binding domain will be able to specifically bind to the various antigens carrying the epitope. In certain embodiments, an antibody that immunospecifically binds to CEA does not bind to carcinoembryonic antigen-related cell adhesion proteins. In certain embodiments, an antibody that immunospecifically binds to CEA does not bind to CEACAM1, CEACAM3, CEACAM4, CEACAM6, CEACAM7 and CEACAM8. In certain embodiments, an antibody that immunospecifically binds to CEA does not bind to at least one of the proteins chosen from: CEACAM1, CEACAM3, CEACAM4, CEACAM6, CEACAM7 and CEACAM8. Note that the ability of a diagnostic or therapeutic reagent to bind to both soluble and membrane bound target CEA is not evidence of lack of immunospecificity. In fact, it is often preferable that an immunospecific reagent immunospecifically bind to both soluble and membrane bound target CEA. Moreover, the ability of a diagnostic or therapeutic reagent to bind to both a mature target CEA and the pro-protein corresponding to that mature target CEA is not considered lack of immunospecificity.

As used herein, the term “A5B7” refers to a mouse monoclonal antibody immunospecific for CEA and described in, for example, WO07/071,426, Int J Cancer (1988) 3: 34-37; British J Cancer (1994) 69: 307-314; Clin Cancer Res (2008) 14: 2639-2646; British J Cancer (1986) 54: 75-82; Cancer Res (1992) 52: 2329-2339.

As used herein, the term “MEDI-565” refers to a bispecific single chain antibody of the BiTE class that includes an anti-CEA binding portion and an anti-CD3 binding portion. The anti-CEA binding portion is a humanized scFv derived from mouse monoclonal antibody A5B7. MEDI-565 is described and disclosed in WO07/071,426, Lutterbuese et al., 2009, Journal of Immunother 32: 341-352, and Osada et al. 2010, British Journal of Cancer, 102: 124-33. The term “BiTE”, when referring to a class of antibody or antibody-like molecules refers to bi-specific T-cell engagers. Such molecules have a portion that is immunospecific for an antigen associated with a diseased state (e.g., an antigen expressed on cancerous cells) and a portion that links such a diseased cell to T cells. WO07/071,426 provides additional exemplary description of BiTE type molecules.

(iii) Diagnostic and Prognostic Methods of Use

Antibodies, antigen binding fragments or immunoglobulin-like molecules disclosed herein are useful in diagnostic and prognostic evaluation of diseases and disorders, particularly cancers associated with CEA expression. At each stage of disease, antibodies may be used to improve diagnostic accuracy and facilitate treatment decisions. Unlike standard diagnostic methods for tumors and cancer, such as computed topographic (CT) scans, which depend on a change in size or architecture of organs or lymph nodes, labeled antibodies can detect abnormal cells at an early stage, because of their expression of tumor antigens, such as CEA or the shedding of such antigens into bodily fluids. Once cancer is diagnosed, accurate staging is important in deciding on the most appropriate therapy. Later, during follow-up post surgery or during or following other treatment, rising levels of tumor antigens in bodily fluids, such as serum and feces, may indicate recurrence before it can be detected by conventional methods. These methods may be used with any of the antibodies, antigen binding fragments or immunoglobulin-like molecules described herein such as antibodies that immunospecifically bind to a specific target form of CEA and do not immunospecifically bind to other forms of CEA.

In certain aspects, the disclosure provides methods of detecting recurrence of a CEA expressing cancer. Patients who have previously been diagnosed with and treated for a CEA expressing cancer can be tested for recurrence of the cancer by detecting a concentration of full-length CEA protein. This method can be repeated over time comparing the concentration of full-length CEA protein found to the previous results for that patient. These methods may be used with any of the antibodies, antigen binding fragments or immunoglobulin-like molecules (e.g., diagnostic reagents) described herein that immunospecifically bind to full-length CEA and do not immunospecifically bind to short form CEA. When the diagnostic step is performed multiple times, it is contemplated that detecting can be with the same diagnostic reagent or with a diagnostic reagent that binds the same or substantially the same epitope. Similarly, it is contemplated that CEA concentrations can be detected in a single type of biological sample (e.g., serum or feces) or that CEA concentrations can be measured in more than one type of biological sample.

In certain aspects, the disclosure provides methods of determining susceptibility to anti-CEA cancer therapy. Patients who have previously been diagnosed with a CEA expressing cancer can be tested for susceptibility to anti-CEA cancer therapy by detecting the concentration of full-length CEA protein. In certain embodiments, the method further provides comparing the concentration of full-length CEA protein to a standard reflecting the standard concentration of full-length CEA protein in samples from healthy subjects. These methods may be used with any of the antibodies, antigen binding fragments or immunoglobulin-like molecules described herein (e.g., diagnostic reagents), such as antibodies that immunospecifically bind to a specific target form of CEA and do not immunospecifically bind to other forms of CEA. When the diagnostic step is performed multiple times, it is contemplated that detecting can be with the same diagnostic reagent or with a diagnostic reagent that binds the same or substantially the same epitope. Similarly, it is contemplated that CEA concentrations can be detected in a single type of biological sample (e.g., serum or feces) or that CEA concentrations can be measured in more than one type of biological sample.

In certain aspects, the disclosure provides methods of monitoring anti-CEA cancer therapy. Patients who have previously been diagnosed with a CEA expressing cancer and are undergoing treatment can be monitored by detecting the concentration of full-length CEA protein. In certain embodiments, the method further provides comparing the concentration of full-length CEA protein to previous concentrations determined for the same patient either before or at an earlier point in treatment. These methods may be used with any of the antibodies, antigen binding fragments or immunoglobulin-like molecules described herein, such as antibodies that immunospecifically bind to a specific target form of CEA and do not immunospecifically bind to other forms of CEA. When the diagnostic step is performed multiple times, it is contemplated that detecting can be with the same diagnostic reagent or with a diagnostic reagent that binds the same or substantially the same epitope. Similarly, it is contemplated that CEA concentrations can be detected in a single type of biological sample (e.g., serum or feces) or that CEA concentrations can be measured in more than one type of biological sample.

In certain aspects, the disclosure provides methods of monitoring an anti-CEA treatment or determining susceptibility to an anti-CEA treatment by using an antibody that binds immunospecifically to the same or substantially the same epitope of CEA as the cancer therapeutic used or being considered for that patient. In certain embodiments, the anti-CEA therapeutic binds immunospecifically to a target form of CEA and does not bind immunospecifically to other forms of CEA.

In certain aspects, the disclosure provides methods for determining a ratio of full-length CEA protein concentration to short form CEA protein concentration by detecting a concentration of full-length CEA protein and a concentration of short form CEA protein in a sample from a subject. In certain embodiments, the method further provides comparing the ratio to a standard reflecting the standard ratio of full-length CEA protein concentration to short form CEA protein concentration in samples from healthy subjects. In certain embodiments, a ratio that varies significantly from the standard ratio is indicative of presence of a CEA-expressing cancer. In certain embodiments, a ratio that varies significantly from the standard ratio is indicative of susceptibility to an anti-CEA therapeutic. In certain embodiments, a change in the ratio after treatment indicates the effectiveness of the therapy. In certain embodiments, the anti-CEA therapeutic binds immunospecifically to a target form of CEA and does not bind immunospecifically to other forms of CEA. In certain embodiments, detecting a concentration of full-length CEA protein is done by contacting a sample with an antibody, an antigen binding fragment or an immunoglobulin-like molecule that immunospecifically binds to full-length CEA protein but does not immunospecifically bind to short form CEA protein. In certain embodiments, detecting a concentration of short form CEA protein is done by contacting a sample with an antibody, an antigen binding fragment or an immunoglobulin-like molecule that immunospecifically binds to short form CEA protein but does not immunospecifically bind to full-length CEA protein.

In certain aspects, the disclosure provides methods for determining a ratio of RNA encoding full-length CEA protein concentration to RNA encoding short form CEA protein concentration by detecting a concentration of RNA encoding full-length CEA protein and a concentration of RNA encoding short form CEA protein in a sample from a subject. In certain embodiments, the method further provides comparing the ratio to a standard reflecting the standard ratio of RNA encoding full-length CEA protein concentration to RNA encoding short form CEA protein concentration in samples from healthy subjects. In certain embodiments, a ratio that varies significantly from the standard ratio is indicative of presence of a CEA-expressing cancer. In certain embodiments, a ratio that varies significantly from the standard ratio is indicative of susceptibility to an anti-CEA therapeutic. In certain embodiments, a change in the ratio after treatment indicates the effectiveness of the therapy. In certain embodiments, the anti-CEA therapeutic binds immunospecifically to a target form of CEA and does not bind immunospecifically to other forms of CEA. In certain embodiments, detecting a concentration of full-length CEA protein is done using primers and or probes specific for RNAs encoding full-length and short from CEA proteins.

Methods of diagnosis can be performed in vitro (ex vivo) using a biological sample (e.g., blood sample, lymph node biopsy or tissue) from a patient or can be performed by in vivo imaging. In certain embodiments, the biological sample (also referred to as a ‘sample’) is chosen from: whole blood, serum, plasma, saliva, urine, feces, seminal plasma, sweat, amniotic fluid, sputum, breast milk, bile, tissue homogenate, and ascites. In certain embodiments, the biological sample is a tumor sample. In certain embodiments, the biological sample is chosen based on the particular CEA-expressing cancer that a patient has or is suspected of having. For example, a sputum sample may, in certain embodiments, be selected if the patient has lung cancer. By way of further example, a feces sample may, in certain embodiments, be selected if the patient has colon or rectal cancer. However, in other embodiments, a whole blood, serum, or plasma sample is examined, regardless of the type of cancer.

In particular embodiments, the present application provides an antibody conjugate wherein the antibodies, or other immunospecific diagnostic reagents of the present disclosure, are conjugated to a diagnostic imaging agent. Compositions comprising the immunospecific diagnostic reagents of the present application can be used to detect target CEA, for example, by radioimmunoassay, ELISA, flow cytometry analysis (including, but not limited to, FACS), immunocytochemistry, immunohistochemistry, etc. One or more detectable labels can be attached to the diagnostic reagent antibodies or to a secondary antibody used to detect the primary diagnostic reagents. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques. In certain embodiments, the labeling moiety is a fluorescent molecule.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include iodine (¹³¹I or ¹²⁵I), indium (¹¹¹In), technetium (⁹⁹Tc), phosphorus (³²P), carbon (¹⁴C), and tritium (³H), or one of the therapeutic isotopes listed above.

The radiolabeled antibody can be administered to a patient where it is localized to cancer cells bearing the antigen with which the antibody reacts, and is detected or “imaged” in vivo using known techniques such as radionuclear scanning using e.g., a gamma detector or emission tomography. See e.g., Bradwell et al., “Developments in Antibody Imaging”, Monoclonal Antibodies for Cancer Detection and Therapy, Baldwin et al., (eds.), pp. 65-85 (Academic Press 1985); Wu and Olafsen (2008) Cancer J 14: 191-197, which are hereby incorporated by reference. Alternatively, a positron emission transaxial tomography scanner, such as designated Pet VI located at Brookhaven National Laboratory, can be used where the radiolabel emits positrons (e.g., ¹¹C, ¹⁸F, ¹⁵O and ¹³N). Additional exemplary information on positron scanning technology can be found, for example, in Gambhir (2002) Nature Reviews 2: 683-693.

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

A label conjugated to a diagnostic and/or therapeutic reagent, and used in the present methods and compositions described herein, is any chemical moiety, organic or inorganic, that exhibits an absorption maximum at wavelengths greater than 280 nm, and retains its spectral properties when covalently attached to an antibody. Labels include, without limitation, a chromophore, a fluorophore, a fluorescent protein, a phosphorescent dye, a tandem dye, a particle, a hapten, an enzyme and a radioisotope.

In certain embodiments, the anti-target CEA antibodies (diagnostic reagents and/or therapeutic reagents) are conjugated to a fluorophore. As such, fluorophores used to label antibodies of the disclosure include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Pat. Nos. 6,977,305 and 6,974,873), a carbocyanine (including any corresponding compounds in U.S. Ser. Nos. 09/557,275; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343; 5,227,487; 5,442,045; 5,798,276; 5,846,737; 4,945,171; U.S. Ser. Nos. 09/129,015 and 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in 5,242,805), aminooxazinones, diaminooxazines, and their benzo-substituted analogs.

In a specific embodiment, the fluorophores conjugated to the diagnostic or therapeutic reagents described herein include xanthene (rhodol, rhodamine, fluorescein and derivatives thereof) coumarin, cyanine, pyrene, oxazine and borapolyazaindacene. In other embodiments, such fluorophores are sulfonated xanthenes, fluorinated xanthenes, sulfonated coumarins, fluorinated coumarins and sulfonated cyanines. Also included are dyes sold under the tradenames, and generally known as, Alexa Fluor, DyLight, Cy Dyes, BODIPY, Oregon Green, Pacific Blue, IRDyes, FAM, FITC, and ROX.

The choice of the fluorophore attached to the anti-target CEA diagnostic and/or therapeutic reagent will determine the absorption and fluorescence emission properties of the conjugated reagent. Physical properties of a fluorophore label that can be used for antibody and antibody bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. In certain embodiments, the fluorophore has an absorption maximum at wavelengths greater than 480 nm. In other embodiments, the fluorophore absorbs at or near 488 nm to 514 nm (particularly suitable for excitation by the output of the argon-ion laser excitation source) or near 546 nm (particularly suitable for excitation by a mercury arc lamp). In other embodiments, a fluorophore can emit in the NIR (near infra red region) for tissue or whole organism applications. Other desirable properties of the fluorescent label may include cell permeability and low toxicity, for example if labeling is to be performed in a cell or an organism (e.g., a living animal).

In certain embodiments, an enzyme is a label and is conjugated to an anti-target CEA diagnostic and/or therapeutic reagents. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art.

In one embodiment, colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

In another embodiment, a colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl)phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. In one embodiment, fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another embodiment, haptens such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently, a peroxidase substrate is added to produce a detectable signal.

Haptens also include hormones, naturally occurring and synthetic drugs, pollutants, allergens, effector molecules, growth factors, chemokines, cytokines, lymphokines, amino acids, peptides, chemical intermediates, nucleotides and the like.

In certain embodiments, fluorescent proteins may be conjugated to the antibodies as a label. Examples of fluorescent proteins include green fluorescent protein (GFP) and the phycobiliproteins and derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorphore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

The present application provides for a method of detecting a cancer comprising detecting the differential expression of mRNA or protein of CEA in said cancer cells in a subject in need of such detection. In one exemplary embodiment, the method of detecting cancer comprising: a) isolating a sample from a patient; b) contacting cells of said sample with the immunospecific diagnostic reagents of the present application; c) contacting non-cancerous cells of the same type of said sample cells with the immunospecific diagnostic reagents of the present application; and d) detecting and comparing the difference of expression of CEA in said sample cells with the non-cancerous cells.

In certain embodiments, antibody conjugates for diagnostic use in the present application are intended for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds. Moreover, it is readily appreciated that any of the fluorophores, radioactive moieties and the like can similarly be used in the context of in vitro diagnostic assays.

In certain embodiments the diagnostic methods of the application may be used in combination with other cancer diagnostic tests.

The present application also provides for a diagnostic kit comprising anti-CEA immunospecific diagnostic reagents. Such a diagnostic kit may further comprise a packaged combination of reagents in predetermined amounts with instructions for performing the diagnostic assay. Where the immunospecific diagnostic reagent is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

In another aspect, the present disclosure concerns immunoassays for binding, purifying, quantifying and otherwise generally detecting CEA protein components. As detailed below, immunoassays, in their most simple and direct sense, are binding assays. In certain embodiments, immunoassays are the various types of enzyme linked immunoadsorbent assays (ELISAs) and radioimmunoassays (RIA) known in the art. Immunohistochemical detection using tissue sections is also particularly useful. However, it will be readily appreciated that detection is not limited to such techniques, and Western blotting, dot and slot blotting, flow cytometry analyses, and the like may also be used.

The steps of various useful immunoassays have been described in the scientific literature, such as, e.g., Nakamura et al., in Enzyme Immunoassays: Heterogeneous and Homogeneous Systems, Chapter 27 (1987), incorporated herein by reference.

In general, the immunobinding methods include obtaining a sample suspected of containing a protein or peptide, in this case, CEA and contacting the sample with a first antibody immunoreactive with CEA under conditions effective to allow the formation of immunocomplexes.

Immunobinding methods include methods for purifying CEA proteins, as may be employed in purifying protein from patients' samples or for purifying recombinantly expressed protein. They also include methods for detecting or quantifying the amount of CEA in a tissue sample or other biological sample, which requires the detection or quantification of any immune complexes formed during the binding process.

The biological sample analyzed may be any sample that is suspected of containing CEA such as a homogenized neoplastic tissue sample. Contacting the chosen biological sample with the immunospecific diagnostic reagent (under conditions effective and for a period of time sufficient to allow the formation of primary immune complexes) is generally a matter of adding the immunospecific diagnostic reagent to the sample and incubating the mixture for a period of time long enough for the immunospecific diagnostic reagents to form immune complexes with, i.e., to bind to, any target CEA present. The sample-antibody composition is washed extensively to remove any non-specifically bound species, allowing only those immunospecific diagnostic reagents specifically bound within the primary immune complexes to be detected.

In general, the detection of immunocomplex formation is well known in the art and may be achieved through the application of numerous approaches. These methods are based upon the detection of radioactive, fluorescent, biological or enzymatic tags. Of course, one may find additional advantages through the use of a secondary binding ligand such as a second antibody or a biotin/avidin ligand binding arrangement, as is known in the art.

The anti-CEA immunospecific diagnostic reagents used in the detection may itself be conjugated to a detectable label, wherein one would then simply detect this label. The amount of the primary immune complexes in the composition would, thereby, be determined.

Alternatively, the first immunospecific diagnostic reagent that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are washed extensively to remove any non-specifically bound labeled secondary antibodies or ligands, and the remaining label in the secondary immune complex is detected.

Particularly for embodiments for which detection of both full-length and short form are desirable (e.g., when evaluating the ratio of full-length to short form), it is contemplated to measure concentration of both proteins in the same assay by, for example, using different detectable labels to detect the concentration of full-length CEA protein and short form CEA protein. However, it is also contemplated that the two forms of CEA protein can be evaluated separately, such as in two aliquots of the same patient sample.

An enzyme linked immunoadsorbent assay (ELISA) is a type of binding assay. In one type of ELISA, anti-CEA immunospecific diagnostic reagents used in the diagnostic method of this application are immobilized onto a selected surface exhibiting protein affinity, such as a well in a polystyrene microtiter plate. Then, a suspected neoplastic tissue sample is added to the wells. After binding and washing to remove non-specifically bound immune complexes, the bound target CEA may be detected. Detection is generally achieved by the addition of another anti-CEA antibody, which need not be immunospecific exclusively for target CEA, that is linked to a detectable label. This type of ELISA is a simple “sandwich ELISA.” Detection may also be achieved by the addition of a second anti-CEA antibody, followed by the addition of a third antibody that has binding affinity for the second antibody, with the third antibody being linked to a detectable label.

In another type of ELISA, the neoplastic tissue samples are immobilized onto the well surface and then contacted with the anti-CEA immunospecific diagnostic reagents used in this application. After binding and washing to remove non-specifically bound immune complexes, the bound anti-CEA immunospecific diagnostic reagents are detected. Where the initial anti-CEA immunospecific diagnostic reagents are linked to a detectable label, the immune complexes may be detected directly. Alternatively, the immune complexes may be detected using a second antibody that has binding affinity for the first anti-CEA immunospecific diagnostic reagents, with the second antibody being linked to a detectable label.

Irrespective of the format employed, ELISAs have certain features in common, such as coating, incubating or binding, washing to remove non-specifically bound species, and detecting the bound immune complexes.

The radioimmunoassay (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an immunospecific diagnostic reagent. Then the free antigen is separated from the immunospecific diagnostic reagents and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

The foregoing are merely exemplary of particular diagnostic techniques that can be used to detect concentration of target CEA in a biological sample. The immunospecific diagnostic reagents of the present disclosure immunospecifically bind to target CEA but do not immunospecifically bind to non-target forms of CEA.

(iv) Therapeutic Methods of Uses

In certain embodiments, the anti-CEA therapeutics and compositions thereof of the disclosure may be administered for prevention and/or treatment of cancer. The disclosure encompasses methods of preventing, treating, maintaining, ameliorating, or inhibiting a CEA-mediated disease or disorder, wherein the methods comprise administering anti-CEA therapeutics. In certain embodiments, the anti-CEA therapeutic binds immunospecifically to a target form of CEA and does not bind immunospecifically to other forms of CEA. In certain embodiments, the therapeutic is an antibody. In certain embodiments, the anti-CEA antibodies bind immunospecifically to a target form of CEA and do not bind immunospecifically to other forms of CEA.

As used herein, an “effective amount” or “therapeutically effective amount” of a pharmaceutical composition of the disclosure in the context of epithelial or other tumors refers to that amount of the therapeutic agent sufficient to destroy, modify, control or remove primary, regional or metastatic tumor tissue. A therapeutically effective amount may refer to the amount of therapeutic agent sufficient to delay or minimize the spread of the epithelial tumor(s). A therapeutically effective amount may also refer to the amount of the therapeutic agent or pharmaceutical agent that provides a therapeutic benefit in the treatment or management of the epithelial tumor(s). Further, a therapeutically effective amount with respect to a therapeutic agent or pharmaceutical agent of the disclosure means that amount of therapeutic agent or pharmaceutical agent alone, or in combination with other therapies, that provides a therapeutic benefit in the treatment or management of an epithelial tumor. Used in connection with an amount of an antibody of the disclosure, the term can encompass an amount that improves overall therapy, reduces or avoids unwanted effects, or enhances the therapeutic efficacy of or synergizes (as defined herein) with another therapeutic agent. In certain embodiments, a therapeutically effective amount of a therapeutic improves overall therapy, reduces or avoids unwanted effects, or enhances the therapeutic efficacy of or synergizes with another therapeutic agent in the treatment of (an) epithelial tumor(s). For example, an anti-CEA antibody may cause a shrinkage of the diameter of an epithelial tumor of 20% if administered to a patient as a mono-therapy. In contrast, a second therapeutic e.g. an anti-cancer agent as defined below, may cause a tumor shrinkage of 10%. However, if both the anti-CEA antibody and said second therapeutic are administered in combination in form of a co-therapy, a tumor shrinkage of 50% may be observed. Such an effect is understood as a synergistic effect as used herein. As referred to herein, the term “therapy” refers to any administration scheme, method and/or agent that can be used in the prevention, treatment or amelioration of an epithelial tumor. The term “prevention, treatment or amelioration of an epithelial tumor” is set forth in more detail below. The terms “therapies” and “therapy” may refer to a biological therapy, supportive therapy, chemotherapy, radiation therapy and/or other therapies useful in treatment, prevention, or amelioration of an epithelial tumor, or one or more symptoms thereof.

As used herein, the terms “treat”, “treatment” and “treating” in the context of administering a therapy or therapies to a patient refer to the reduction or amelioration of the progression, severity, and/or duration of an epithelial tumor. Said epithelial tumor(s) may be associated with aberrant expression e.g., overexpression or activity of CEA, and/or the amelioration of one or more symptoms thereof resulting from the administration of one or more therapies (including the administration of one or more pharmaceutical or therapeutic agents).

Treatment can encompass administering therapeutic agents of the present disclosure via oral administration, topical administration, via injection, intranasally, rectally, transdermally, via an impregnated or coated device such as an ocular insert, catheter, wire or implant, or iontophoretically, amongst other routes of administration.

Note that, where context indicates, treatment is also used interchangeably with therapeutic regimen or therapy. In other words, the term treatment can refer to all or a portion of a patient's therapeutic regimen. In certain embodiments, such therapeutic regimen includes administration of an anti-CEA cancer therapeutic that immunospecifically binds a target CEA. Such treatment may also include one or more additional therapeutic modalities alone or in addition to an anti-CEA cancer therapeutic. Such one or more additional therapeutic modalities include, but are not limited to, surgery, chemotherapy, radiation therapy, acupuncture, nutritional therapy, herbal therapy and the like. In certain embodiments, treatment includes administration of an anti-CEA cancer therapeutic that immunospecifically binds a target CEA, and the patient is followed diagnostically with a diagnostic reagent that immunospecifically binds the same or substantially the same epitope as the anti-CEA cancer therapeutic. In certain embodiments, the diagnostic reagent and the anti-CEA cancer therapeutic are the same antibody or share at least one antigen binding domain. In certain embodiments, the diagnostic reagent is not MEDI-565 and/or a variant thereof, but the therapeutic reagent is MEDI-565 and/or a variant thereof.

For administration via injection, the anti-CEA therapeutic can be injected intraocularly, periocularly, intramuscularly, intra-arterially, subcutaneously, or intravenously. A pump mechanism may be employed to administer the anti-CEA therapeutic over a preselected period. For some embodiments of the disclosure it is desirable to deliver drug locally, thus injections may be made periocularly, intraocularly, intravitreally, subconjunctively, retrobulbarly, into the sclera, or intercamerally. For some embodiments of the disclosure, systemic delivery is preferred. In certain embodiments, administration is an intravenous administration over a given time/time period.

For systemic administration, the anti-CEA therapeutic can be formulated for and administered orally. For administration that may result in either regional or systemic distribution of the therapeutic agents, the composition of the disclosure may be administered intranasally, transdermally, by inhalation or via some forms of oral administration, e.g. with use of a mouthwash or lozenge incorporating a compound of the disclosure that is poorly absorbed from the G.I. tract. For administration that may result in regional or local delivery of the composition of the disclosure, iontophoretic or topical administration may be used.

While the anti-CEA therapy may be administered alone, in certain embodiments administration is in a pharmaceutically acceptable carrier. Examples of suitable pharmaceutical carriers are well known in the art and include phosphate buffered saline solutions, water, liposomes, various types of wetting agents, sterile solutions, etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Preparations for parenteral administration include sterile aqueous or non-aqueous solutions, and suspensions. Examples of non-aqueous solvents are propylene glycol, polyethylene glycol, and injectable organic esters such as ethyl oleate. Aqueous carriers include water, aqueous solutions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the composition might comprise proteinaceous carriers, like, e.g., serum albumin or immunoglobulin, in certain embodiments of human origin. It is envisaged that the co-therapy might comprise, in addition to the anti-CEA therapy further biologically active agents, depending on the intended use of the pharmaceutical composition. Such agents might be agents acting on the gastrointestinal system, agents acting as cytostatica, agents preventing hyperurikemia, agents inhibiting immune reactions (e.g. corticosteroids, FK506), drugs acting on the circulatory system and/or agents such as T-cell co-stimulatory molecules or cytokines known in the art. In certain embodiments, the anti-CEA therapy is formulated in a buffer, a stabilizer and a surfactant. The buffer may be a phosphate, citrate, succinate or acetate buffer. The stabilizer may be (an) amino acid(s) and/or a sugar. The surfactants may be detergents, PEGs, or the like. In certain embodiments, the anti-CEA therapy is formulated in citrate, lysine, trehalose and Tween 80. As a diluent for said pharmaceutical composition, isotonic saline and Tween 80 is preferred.

The term “amelioration” as used herein refers to an improvement or a moderation in the severity of a disease, i.e. an epithelial tumor. For example, such an amelioration may be the achievement of a stable disease—or even more preferred—a shrinkage of the epithelial tumor(s), i.e. a minimal, partial response or complete response, due to the administration of the pharmaceutical compositions of the disclosure. “Stable disease” refers to a disease state in which no or no significant tumor progression/growth can be observed or detected by clinical and/or histological diagnostic methods. For example, a shrinkage of the tumor greater than 50% shrinkage of the sum of cross-sectional areas of index lesions may be considered as a “partial response”. A “complete response” denotes a state in which no lesion(s) can be detected any more after treatment. A response with a tumor shrinkage between stable disease and partial response may be considered as a minimal response. For instance, a 20%, 25% or 30% shrinkage of the sum of cross-sectional areas of index lesions may be referred to as a minimal response. The term “amelioration” as used herein encompasses also a reduction of the number of epithelial tumors. It furthermore denotes the prevention/slowdown of tumor progression. Moreover, an improvement of the overall survival of treated tumor patients in comparison to non-treated tumor patients may be considered as an “amelioration” as used herein. This applies mutatis mutandis to an improvement of the progression-free survival or the relapse-free survival of treated tumor patients as compared to non-treated tumor patients. In addition, the term “amelioration” can also refer to a reduction of the intensity of the symptoms of an epithelial tumor, resulting e.g. in an improvement of the quality of life of the treated tumor patients.

The term “prevention of an epithelial tumor” as used herein is to be understood as follows: After surgical removal of the primary epithelial tumor(s) from a human patient and/or after chemotherapeutic or radiological treatment of the primary epithelial tumor(s), it may be the case that not all tumor cells could be eliminated from the body. However, these remaining tumor cells may give rise to recurrent cancer, i.e. local recurrence and/or metastases in the patient. Metastasis is a frequent complication of cancer, yet the process through which cancer cells disseminate from the primary tumor(s) to form distant colonies is poorly understood. Metastatic cancers are almost without exception uncurable raising the necessity for new therapeutic modalities. The pharmaceutical composition of the disclosure can be used to kill these disseminated tumor cells in order to prevent the formation of secondary tumors (originating from the tumor cells remaining in the body after primary therapy). In this way, the pharmaceutical composition helps to prevent the formation of local recurrence and/or metastases in tumor patients.

The success of the anti-tumor therapy may be monitored by established standard methods for the respective disease entities, e.g. by computer-aided tomography, X-ray, nuclear magnetic resonance tomography (e.g. for National Cancer Institute—criteria based response assessment [Cheson (1999), J. Clin. Oncol.; 17(4):1244]), positron-emission tomography scanning, endoscopy, Fluorescence Activated Cell Sorting, aspiration of bone marrow, pleural or peritoneal fluid, tissue/histologies, and various epithelial tumor specific clinical chemistry parameters (e.g. soluble CEA concentration in serum) and other established standard methods may be used. In addition, assays determining T cell activation may be used; see e.g. WO99/054440. Statistics for the determination of overall survival, progression-free survival or relapse-free survival of treated tumor patients in comparison to non-treated tumor patients may also be used.

In certain embodiments, said epithelial tumor is a gastrointestinal adenocarcinoma, a breast adenocarcinoma or a lung adenocarcinoma. In certain embodiments, said gastrointestinal adenocarcinoma is a colorectal, pancreatic, an oesophageal or a gastric adenocarcinoma.

In other embodiments, said pharmaceutical composition of the disclosure is for the treatment of progressive tumors, late stage tumors, tumor patients with high tumor load/burden, metastatic tumors, or tumor patients with a CEA serum concentration higher than 100 ng/ml (as determined e.g. by ELISA).

In another embodiment of the uses or methods of the disclosure, said pharmaceutical composition as defined hereinabove is suitable to be administered in combination with an additional drug, i.e. as part of a co-therapy.

In certain embodiments, the anti-CEA antibody or therapeutic pharmaceutical composition is administered in combination with one or more other therapies. In certain embodiments, the anti-CEA antibody or therapeutic pharmaceutical composition is administered to a patient concurrently with one or more other therapies. In certain embodiments, such therapies are useful for the treatment of epithelial tumors. The term “concurrently” is not limited to the administration of pharmaceutical compositions or therapeutic agents at exactly the same time, but rather it is meant that the anti-CEA antibody or therapeutic pharmaceutical composition and the other agent(s) are administered to a patient in a sequence and within a time interval such that the anti-CEA antibody or therapeutic pharmaceutical composition can act together with the other agent to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route. In other embodiments, the anti-CEA antibody or therapeutic pharmaceutical composition are administered before, concurrently or after surgery. In certain embodiments the surgery completely removes localized epithelial tumors or reduces the size of large epithelial tumors. Surgery can also be done as a preventive measure or to relieve pain. The dosage amounts and frequencies of administration provided herein are encompassed by the term “therapeutically effective” as defined above. The dosage and frequency further will typically vary according to factors specific for each patient depending on the specific therapeutic or prophylactic agents administered, the severity and type of epithelial tumor, the route of administration, as well as age, body weight, response, and the past medical history of the patient. Suitable regimens can be selected by one skilled in the art by considering such factors and by following, for example, dosages reported in the literature and recommended in the Physicians' Desk Reference (59th ed., 2005).

In some embodiments, therapy by administration of the anti-CEA antibody or therapeutic pharmaceutical composition is combined with the administration of one or more therapies such as chemotherapies, radiation therapies, hormonal therapies, and/or biological therapies/immunotherapies. Therapeutic agents include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including double-stranded or single-stranded DNA, or double-stranded or single-stranded RNA, as well as triple helix nucleic acid molecules. Therapeutic agents can be derived from any known organism (including, but not limited to, animals, plants, bacteria, fungi, and protista, or viruses) or from a library of synthetic molecules.

In a specific embodiment, the methods and uses of the disclosure encompass administration of the anti-CEA antibody or therapeutic pharmaceutical composition combination with the administration of one or more therapeutic agents that are inhibitors of kinases such as Gefitinib (Iressa), Erlotinib (Tarceva), anti-EGFR-antibodies (e.g. Cetuximab; Erbitux), or anti-Her2/neu-antibodies (e.g. Trastuzumab; Herceptin) described in the art; see e.g., Hardie and Hanks (1995) The Protein Kinase Facts Book, I and II, Academic Press, San Diego, Calif.

In another specific embodiment, the methods and uses of the disclosure encompass administration of the anti-CEA antibody or therapeutic pharmaceutical composition in combination with the administration of one or more therapeutic agents that are angiogenesis inhibitors such as anti-VEGF-antibodies (e.g. Bevacizumab; Avastin), small molecular compounds (e.g. Vatalanib or Sorafenib) or COX-inhibitors described in the art.

In another specific embodiment, the methods and uses of the disclosure encompass administration of the anti-CEA antibody or therapeutic pharmaceutical composition in combination with the administration of one or more therapeutic agents that are anti-cancer agents such as 5-Fluorouracil, Leucovorin, Capecitabine, Oxaliplatin, Irinotecan, Gemcitabine, Doxorubicin, Epirubicin, Etoposide, Cisplatin, Carboplatin, Taxanes (e.g. Docetaxel, Paclitaxel) described in the art.

In certain embodiments, a co-therapy of a patient with an epithelial tumor using an anti-CEA antibody or therapeutic pharmaceutical composition in combination with (a) further therapeutic agent(s) results in a synergistic effect. As used herein, the term “synergistic” refers to a combination of therapies (e.g., a combination of an anti-CEA antibody or therapeutic and (a) further therapeutic agent(s) as set forth above) which is more effective than the additive effects of any two or more single therapies (e.g., one or more therapeutic agents). For example, an anti-CEA antibody or therapeutic may cause a shrinkage of the diameter of an epithelial tumor of 20% if administered to a patient as a mono-therapy. In contrast, a second therapeutic e.g. an anti-cancer agent as defined below, may cause a tumor shrinkage of 10%. However, if both the anti-CEA antibody or therapeutic and said second therapeutic are administered in combination in form of a co-therapy, a tumor shrinkage of 50% may be observed.

A synergistic effect of a combination of therapies (e.g., a combination of an anti-CEA antibody or therapeutic and (a) further therapeutic agent(s) as set forth above) permits the use of lower dosages of one or more of therapies (e.g., one or more therapeutic agents) and/or less frequent administration of said therapies to a patient with a disease, e.g. an epithelial tumor. The ability to utilize lower dosages of therapies (e.g., therapeutic agents) and/or to administer said therapies less frequently reduces the toxicity associated with the administration of said therapies to a subject without reducing the efficacy of said therapies in the prevention or treatment of a disease, e.g. an epithelial tumor. In addition, a synergistic effect can result in improved efficacy of therapies (e.g., therapeutic agents) in the prevention, management, treatment and/or amelioration of an epithelial tumor (which may be associated with aberrant expression (e.g., overexpression) or activity of CEA). Finally, synergistic effect of a combination of therapies (e.g., therapeutic agents) may avoid or reduce adverse or unwanted side effects associated with the use of any single therapy.

In said co-therapy, an active agent may be optionally included in the same pharmaceutical composition as the anti-CEA antibody or therapeutic, or may be included in a separate pharmaceutical composition. In this latter case, said separate pharmaceutical composition is suitable for administration prior to, simultaneously as or following administration of said pharmaceutical composition comprising the anti-CEA antibody or therapeutic. The additional drug or pharmaceutical composition may be a non-proteinaceous compound or a proteinaceous compound. In the case that the additional drug is a proteinaceous compound, it is advantageous that the proteinaceous compound be capable of providing an activation signal for immune effector cells.

In certain embodiments, said proteinaceous compound or non-proteinaceous compound may be administered simultaneously or non-simultaneously with an anti-CEA antibody or therapeutic. In certain embodiments, said subject to be treated is a human.

Cancer therapies and their dosages, routes of administration and recommended usage are known in the art and have been described in such literature as the Physician's Desk Reference (56th ed., 2002).

In certain embodiments, the therapeutic regimen comprises treatment with a bispecific antibody (including a bispecific single chain antibody) that includes both an anti-CEA portion and an anti-CD3 portion. In certain embodiments, the therapeutic to be used with the methods of the disclosure is MEDI-565. Specific methods for treating with such bispecific antibodies, including MEDI-565, are found in PCT publication WO2007/071426, incorporated herein by reference in its entirety. See also, Lutterbuese et al., 2009, Journal of Immunotherapy 32: 341-352, Osada et al. 2010, British Journal of Cancer, 102: 124-33, and Medical News Today (http://www.medicalnewstoday.com/articles/145690.php), each of which describe MEDI-565 and are incorporated by reference in their entirety. In certain embodiments, the therapeutic to be used includes, at least, a CEA binding portion that binds to the same or substantially the same epitope as MEDI-565.

In certain embodiments, the therapeutic to be used includes, at least, a CEA binding portion comprising the amino acid sequence represented in any of SEQ ID NOs: 28-44 and 46-51. In certain embodiments, the therapeutic to be used is a bispecific antibody comprising the amino acid sequence represented in any of SEQ ID NOs: 28-44 and 47. In certain embodiments, the therapeutic to be used is a bispecific antibody comprising the amino acid sequence represented in any of SEQ ID NOs: 34, 36, 41, 42, 43, and 47. In certain embodiments, the therapeutic to be used is a bispecific antibody comprising the amino acid sequence represented in any of SEQ ID NOs: 37-40. In certain embodiments, the therapeutic to be used is a bispecific antibody comprising the amino acid sequence represented in SEQ ID NO: 48. In certain embodiments, the therapeutic to be used is a bispecific antibody comprising the amino acid sequence represented in SEQ ID NO: 49. In certain embodiments, the therapeutic to be used is a bispecific antibody comprising the amino acid sequence represented in SEQ ID NOs: 48 and 49. In certain embodiments, the therapeutic to be used is a bispecific antibody comprising the amino acid sequence represented in SEQ ID NO: 46.

In certain embodiments, the therapeutic to be used is a bispecific antibody, such as a bispecific single chain antibody. The order of arrangement of the first and second binding domains, such as within the bispecific antibody or bispecific single chain antibody, is relevant. It is envisaged that the arrangement of the binding domains may be VH_{CEA}-VL_{CEA}-VH_{CD3}-VL_{CD3}, VL_{CEA}-VH_{CEA}-VH_{CD3}-VL_{CD3}, VH_{CD3}-VL_{CD3}-VH_{CEA}-VL_{CEA} or VH_{CD3}-VL_{CD3}-VL_{CEA}-VH_{CEA}. In some examples, the first binding domain specifically binding to human CD3 is arranged in the VH-VL orientation. For example, the binding domains of the bispecific single chain antibodies defined herein may be arranged in the order VH_{CEA}-VL_{CEA}-VH_{CD3}-VL_{CD3} or VL_{CEA}-VH_{CEA}-VH_{CD3}-VL_{CD3}. As used in this context, “N-terminally to” or “C-terminally to” and grammatical variants thereof denote relative location within the primary amino acid sequence rather than placement at the absolute N- or C-terminus of a molecule. Hence, as a non-limiting example, a first binding domain which is “located C-terminally to the second binding domain” simply denotes that the first binding domain is located to the carboxyl side of the second binding domain within the bispecific antibody, and does not exclude the possibility that an additional sequence, for example a tag as set forth above, or another proteinaceous or non-proteinaceous compound such as a radioisotope, is located at the ultimate C-terminus of the bispecific antibody.

In certain embodiments, the therapeutic is a bispecific antibody or a single chain bispecific antibody with binding domains arranged in the order VH_{CEA}-VL_{CEA}-VH_{CD3}-VL_{CD3} or VL_{CEA}-VH_{CEA}-VH_{CD3}-VL_{CD3}. In certain embodiments, the arrangement is VL_{CEA}-VH_{CEA}-VH_{CD3}-VL_{CD3}. In certain embodiments, the therapeutic is a bispecific single chain antibody construct A240 VL-B9 VH×SEQ ID NO. 50 VHVL as defined in SEQ ID NO. 46. In some examples, the binding domain specifically binding to human CEA of the bispecific antibody or bispecific single chain antibody comprises at least one CDR, such as a CDR-H3, such as a part of the CDR-H3 of murine monoclonal antibody A5B7 with the amino acid sequence “FYFDY” (SEQ ID NO. 28) corresponding to Kabat positions 100, 100a, 100b, 101, and 102, respectively, of CDR-H3 of murine monoclonal antibody A5B7. In some examples, the CDH-H3 has the amino acid sequence “DX₁X₂X₃X₄FYFDY” (SEQ ID NO. 29), wherein “X₁”, “X₂”, “X₃” or “X₄” represents any amino acid residue, and the amino acid residue “D” corresponds to Kabat position 95 of CDR-H3 of murine monoclonal antibody A5B7 and the amino acid residues “FYFDY” correspond to Kabat positions 100, 100a, 100b, 101, and 102, respectively, of CDR-H3 of murine monoclonal antibody A5B7. Herein, “X₁”, “X₂”, “X₃” and “X₄” correspond to Kabat positions 96 (“X₁”), 97 (“X₂”), 98 (“X₃”) and 99 (“X₄”), respectively, of CDR-H3 of murine monoclonal antibody A5B7. It is envisaged that “X₁”, “X₂”, “X₃” or “X₄” represent amino acid residue “R” (Arginine), “G” (Glycine), “L” (Leucine), “Y” (Tyrosine), “A” (Alanine), “D” (Aspartic acid), “S” (Serine), “W” (Tryptophan), “F” (Phenylalanine) or “T” (Threonine). In certain embodiments, it is excluded from the scope of the disclosure that “X₁”, “X₂”, “X₃” and “X₄” represent the same amino acid, e.g. that “X₁”, “X₂”, “X₃” and “X₄” are all “F” (Phenylalanine). In certain embodiments, “X₁” represents “R” (Arginine), “F” (Phenylalanine), “M” (Methionine), “E” (Glutamic acid), or “T” (Threonine); “X₂” represents “G” (Glycine), “Y” (Tyrosine), “A” (Alanine), “D” (Aspartic acid), or “S” (Serine); “X₃” represents “L” (Leucine), “F” (Phenylalanine), “M” (Methionine), “E” (Glutamic acid), or “T” (Threonine); and “X₄” represents “R” (Arginine), “Y” (Tyrosine), “A” (Alanine), “D” (Aspartic acid), or “S” (Serine).

In some examples, the second binding domain specific for human CEA comprises at least the amino acid sequence “RFYFDY” (SEQ ID NO. 30), “LRFYFDY” (SEQ ID NO. 31), “GLRFYFDY” (SEQ ID NO. 32), or “RGLRFYFDY” (SEQ ID NO. 33) of CDR-H3 of monoclonal antibody A5B7. In some examples, the second binding domain comprises the complete CDR-H3 of A5B7 with the amino acid sequence “DRGLRFYFDY” (SEQ ID NO. 34) corresponding to Kabat positions 95 (“D”, Aspartic acid), 96 (“R”; Arginine), 97 (“G”; Glycine), 98 (“L”; Leucine), 99 (“R”; Arginine), 100 (“F”; Phenylalanine), 100a (“Y”; Tyrosine), 100b (“F”; Phenylalanine), 101 (“D”; Aspartic acid), and 102 (“Y”; Tyrosine), respectively. Numbering according to the Kabat system is set forth e.g. in Kabat, E. A., T. T. Wu, H. M. Perry, K. S. Gottesman, and C. Foeller. 1991. Sequences of Proteins of Immunological Interest, 5th ed. Bethesda, Md.: National Center for Biotechnology Information, National Library of Medicine.

In certain embodiments, it may be desirable to further modify this A5B7-derived “DRGLRFYFDY” (SEQ ID NO: 34) CDR-H3 amino acid sequence e.g. in order to improve affinity for the CEA target antigen (on the epithelial tumor cells) and/or to optimize “fine specificity” of the bispecific single chain antibody as defined herein. To this end, in the amino acid sequence “DX₁X₂X₃X₄FYFDY” (SEQ ID NO. 29)”, various amino acid residues may be tested at positions “X1”, “X2”, “X3” and/or “X4” (corresponding to Kabat positions 96 (“X₁”), 97 (“X₂”), 98 (“X₃”) and 99 (“X₄”), respectively, of CDR-H3 of murine monoclonal antibody A5B7) in order to identify a modified CDR-H3 with improved affinity and/or fine specificity. For instance, “X₁”, “X₂”, “X₃” or “X₄” may represent amino acid residue “R” (Arginine), “G” (Glycine), “L” (Leucine), “Y” (Tyrosine), “A” (Alanine), “D” (Aspartic acid), “S” (Serine), “W” (Tryptophan), “F” (Phenylalanine) or “T” (Threonine). Herein, one, two, three or all four of the indicated “X” positions may be exchanged in comparison to the original “RGLR” amino acid sequence at Kabat positions 96 to 99 in the CDR-H3 “DRGLRFYFDY” (SEQ ID NO. 34) amino acid sequence. In certain embodiments, it is excluded that “X₁”, “X₂”, “X₃” and “X₄” represent the same amino acid, e.g. that “X₁”, “X₂”, “X₃” and “X₄” are all “F” (Phenylalanine). The above-mentioned modification of the A5B7-derived “DRGLRFYFDY” CDR-H3 amino acid sequence can be achieved by methods known in the art, such as PCR using randomized primers, which allows the generation of bispecific single chain antibodies with such modified CDR-H3 regions in the CEA-binding domain. Affinity or fine specificity of these modified bispecific single chain antibodies can be tested by methods described in the art, e.g. by ELISA, Biacore or FACS analysis.

In some embodiments, the binding domain specific for human CEA of the therapeutic agent, such as a bispecific single chain antibody, comprises a CDR-H1 having the amino acid sequence “SYWMH” (SEQ ID NO. 36) and/or a CDR-H2 having the amino acid sequence “FIRNKANGGTTEYMSVKG” (SEQ ID NO. 37) or “FILNKANGGTTEYMSVKG” (SEQ ID NO. 38).

In some embodiments, the binding domain specific for human CEA of the therapeutic agent, such as a bispecific single chain antibody, comprises a CDR-H1 having the amino acid sequence “SYWMH” (SEQ ID NO. 36) and/or a CDR-H2 having the amino acid sequence “FIRNKANGGTTEYMSVKG” (SEQ ID NO. 37) or “FIRNKANGGTTEYAASVKG” (SEQ ID NO. 47).

Alternatively, said second binding domain specific for human CEA of the bispecific single chain antibodies defined herein comprises a CDR-H1 having the amino acid sequence “TYAMH” (SEQ ID NO. 39) and/or a CDR-H2 having the amino acid sequence “LISNDGSNKYYADSVKG” (SEQ ID NO. 40).

In certain embodiments, the amino acid sequence of the VH region of the binding domain specific for human CEA, such as of a bispecific antibody or a bispecific single chain antibody comprises “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO.36) and a CDR-H2 having the amino acid sequence “FIRNKANGGTTEYMSVKG” (SEQ ID NO. 37).

In certain embodiments, the amino acid sequence of the VH region of the binding domain specific for human CEA, such as in a bispecific format or a bispecific single chain format, is SEQ ID NO. 146 comprising “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO. 36) and a CDR-H2 having the amino acid sequence “FILNKANGGTTEYAASVKG” (SEQ ID NO.44).

In certain embodiments, the amino acid sequence of the VH region of the binding domain specific for human CEA, such as in a bispecific format or a bispecific single chain format, comprises “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO. 36) and a CDR-H2 having the amino acid sequence “FIRNKANGGTTEYAASVKG” (SEQ ID NO.47).

In certain embodiments, the amino acid sequence of the VH region of the binding domain specific for human CEA, such as in a bispecific single chain formant, comprises “DRGLRFYFDY” (SEQ ID NO. 34) corresponding to Kabat positions 95-102 of the CDR-H3 of murine monoclonal antibody A5B7 and a CDR-H1 having the amino acid sequence “TYAMH” (SEQ ID NO. 39) and a CDR-H2 having the amino acid sequence “LISNDGSNKYYADSVKG” (SEQ ID NO. 40).

In certain embodiments, the amino acid sequence of the VH region of the binding domain specific for human CEA comprises an amino acid sequence having the sequence of SEQ ID NO: 59.

Thus, said binding domain specific for human CEA of, for example a bispecific single chain antibody, may comprise one, two or three CDR-H regions as defined above.

In certain embodiments, the amino acid sequence of the VL region of the binding domain specific for human CEA, such as in a bispecific format or a bispecific single chain format, comprises CDR-L1 having the amino acid sequence “TLRRGINVGAYSIY” (SEQ ID NO. 41) and a CDR-L2 having the amino acid sequence “YKSDSDKQQGS” (SEQ ID NO. 42 and a CDR-L3 having the amino acid sequence “MIWHSGASAV” (SEQ ID NO. 43).

In certain embodiments, the amino acid sequence of the VH region of the binding domain specific for human CEA comprises an amino acid sequence having the sequence of SEQ ID NO: 48.

As noted above, the order or arrangement of the variable regions of the second binding domain specifically binding to CEA may be VH-VL or VL-VH.

In certain embodiments, the V regions of the CEA binding portion of a therapeutic agent, such as a therapeutic bispecific antibody, or a bispecific single chain antibody is chosen from:

(a) the VH region consists of the amino acid sequence shown in SEQ ID NO. 49 and the VL region consists of the amino acid sequence shown in SEQ ID NO. 48;
(b) the VH region consists of the amino acid sequence shown in SEQ ID NO. 51 and the VL region consists of the amino acid sequence shown in SEQ ID NO. 48;

In certain embodiments, the therapeutic is a bispecific single chain antibody comprising an amino acid sequence chosen from:

(a) an amino acid sequence as depicted in any of SEQ ID NOs. 28-51
(b) an amino acid sequence encoding any of SEQ ID NOs. 28-51
(c) an amino acid sequence encoded by a nucleic acid sequence hybridizing under stringent conditions to the complementary nucleic acid sequence of (b);
(d) an amino acid sequence encoded by a nucleic acid sequence which is degenerate as a result of the genetic code to a nucleotide sequence of (b); and
(e) an amino acid sequence at least 85% identical, at least 90% identical, or at least 95% identical to the amino acid sequence of (a) or (b).

In certain embodiments, the therapeutic is a bispecific single chain antibody comprising the amino acid sequence of SEQ ID NO: 46.

(v) Pharmaceutical Formulations

Formulations particularly useful for antibody-based therapeutic agents are also described in U.S. Patent App. Publication Nos. 20030202972, 20040091490 and 20050158316. In certain embodiments, the liquid formulations of the application are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject antibodies are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

Formulations of the subject therapeutic reagents include those suitable for oral, dietary, topical, parenteral (e.g., intravenous, intraarterial, intramuscular, subcutaneous injection), ophthalmologic (e.g., topical or intraocular), inhalation (e.g., intrabronchial, intranasal or oral inhalation, intranasal drops), rectal, and/or intravaginal administration. Other suitable methods of administration can also include rechargeable or biodegradable devices and controlled release polymeric devices. Stents, in particular, may be coated with a controlled release polymer mixed with an agent of the application. The pharmaceutical compositions of this disclosure can also be administered as part of a combinatorial therapy with other agents (either in the same formulation or in a separate formulation). Formulations for diagnostic reagents are within the level of skill in the art and include suitable excipients for in vitro (ex vivo) or in vivo use.

The amount of the formulation which will be therapeutically effective can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula: Dose (mL)=[patient weight (kg)×dose level (mg/kg)/drug concentration (mg/mL)]

To achieve the desired reductions of body fluid parameters, such anti-CEA antibodies can be administered in a variety of unit dosage forms. The dose will vary according to the particular antibody. For example, different antibodies may have different masses and/or affinities, and thus require different dosage levels. Antibodies prepared as Fab′ fragments or single chain antibodies will also require differing dosages than the equivalent native immunoglobulins, as they are of considerably smaller mass than native immunoglobulins, and thus require lower dosages to reach the same molar levels in the patient's blood.

Other therapeutics of the disclosure can also be administered in a variety of unit dosage forms and their dosages will also vary with the size, potency, and in vivo half-life of the particular therapeutic being administered.

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration. The packaging material will include a label which indicates that the formulation is for use in the treatment of cancer.

The efficient dosages and the dosage regimens for the diagnostic reagents of the disclosure depend on the disease or condition to be treated and can be determined by the persons skilled in the art.

(vi) Articles of Manufacture and Kits

The disclosure provides a pharmaceutical pack or kit comprising one or more containers filled with a liquid formulation or lyophilized formulation of the disclosure. In a specific embodiment, the formulations of the disclosure comprise anti-CEA diagnostic or therapeutic reagents recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. In a specific embodiment, the formulations of the disclosure are formulated in single dose vials as a sterile liquid. The formulations of the disclosure may be supplied in 3 cc USP Type I borosilicate amber vials (West Pharmaceutical Services-Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration.

In certain embodiments, kits comprising anti-CEA diagnostic reagents are also provided that are useful for various purposes, e.g., research and diagnostic including for purification or immunoprecipitation of CEA from cells, detection of target CEA, etc. For isolation and purification of CEA, the kit may contain an anti-CEA diagnostic reagent coupled to beads (e.g., sepharose beads). Kits may be provided which contain the antibodies for detection and quantitation of target CEA in vitro, e.g. in an ELISA or a Western blot. As with the article of manufacture, the kit comprises a container and a label or package insert on or associated with the container. The container holds a composition comprising at least one anti-CEA diagnostic reagent of the disclosure. Additional containers may be included that contain, e.g., diluents and buffers, control diagnostic reagents. The label or package insert may provide a description of the composition as well as instructions for the intended in vitro or diagnostic use.

In certain embodiments, the disclosure provides kits that include both an anti-CEA therapeutic reagent and a suitable diagnostic reagent (so called dual reagent kits). Such kits may include any of the foregoing embodiments, such as instructions for use, suitable buffers, etc. In certain embodiments, the dual reagents kits are matched so that the therapeutic reagent and the diagnostic reagent bind the same or substantially the same epitope. For example, the therapeutic reagent and the diagnostic reagent immunospecifically bind a target CEA (full-length or short form), and the diagnostic and therapeutic reagents immunospecifically bind the same or substantially the same epitope. In certain embodiments, the diagnostic reagent and the therapeutic reagent are the same. In certain embodiments, the two reagents share at least one antigen binding portion.

(vii) RNA Detection Methods

The present application provides for a method of detecting expression of full-length an/or short form carcinoembryonic antigen (CEA) RNA in a biological sample. One or both of a nucleic acid probe or nucleic acid primers that hybridize to a CEA nucleotide sequence to specifically identify expression of full-length and/or short form CEA by (i) hybridizing specifically to a sequence present in full-length CEA nucleotide sequence (e.g. the region spliced out of short form) or to short form CEA nucleotide sequence but not to a full-length CEA nucleotide sequence (e.g., a splice junction in short form CEA nucleotide that is absent from full-length) (ii) hybridizing specifically to both short form CEA nucleotide sequence and full-length CEA nucleotide sequence in a manner that distinguishes expression of short form CEA from expression of full-length CEA are provided. RNA from a biological sample is further provided. Finally, the expression of short form CEA RNA in the biological sample is detected by using the nucleic acid probe and/or nucleic acid primers.

Detection of a nucleic acid of interest in a biological sample may optionally be effected by hybridization-based assays using an oligonucleotide probe (non-limiting examples of probes according to the present disclosure were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection). More recently, PNAs have been described (Nielsen et al. 1999, Current Opin. Biotechnol. 10:71-75). Other detection methods include kits containing probes on a dipstick setup and the like.

Hybridization based assays which allow the detection of a target of interest (i.e., DNA or RNA) in a biological sample rely on the use of oligonucleotides which can be, for example, 10, 15, 20, or 30 to 100 nucleotides long, from 10 to 50 or from 40 to 50 nucleotides long.

In certain embodiments, the isolated polynucleotides (oligonucleotides) of the present disclosure are hybridizable with any of the herein described nucleic acid sequences under moderate to stringent hybridization conditions.

Detection of a nucleic acid of interest in a biological sample may also optionally be effected by NAT-based assays, which involve nucleic acid amplification technology, such as PCR (or variations thereof such as real-time PCR, for example).

Examples of detecting RNA expression using different techniques are provided in the Examples section of the instant application. Such examples include the use of primers to amplify RNA via a traditional PCR reaction, as well as the use of a combination of a probe and primers employed in an approach referred to as TaqMan. Further examples of methodology for detecting RNA include quantitative RT-PCR, SAGE, MPSS, array-based methods, and direct sequencing.

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present disclosure, which are selected to be used together in amplifying a selected nucleic acid sequence by one of a number of types of amplification processes, such as a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

Oligonucleotide primers of the present disclosure may be of any suitable length, depending on the particular assay format and the particular needs and targeted genomes employed. Optionally, the oligonucleotide primers are at least 12 nucleotides in length, in certain embodiments between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning-A Laboratory Manual, 2nd Edition, CSH Laboratories; Ausubel et al., 1989, in Current Protocols in Molecular Biology, John Wiley & Sons Inc., N.Y.).

In an alternative embodiment, expression can be detected by direct sequencing of cDNA. Such direct sequencing provides an additional method for detecting and quantitating RNA.

(viii) Diagnostic Methods Using Detection of RNA

In another aspect, the disclosure provides methods of identifying patients that may be susceptible to a cancer therapeutic that immunospecifically binds to a target carcinoembryonic antigen (CEA) protein by evaluating a tumor sample to assess whether the tumor expresses one or both of full-length CEA RNA and/or short form CEA RNA. In certain embodiments, the method entails obtaining a tumor sample from a patient, and detecting expression in the tumor sample of a target CEA RNA. For example, the sample may be contacted with a probe and/or primers that distinguish RNA expression of full-length CEA from RNA expression of short form CEA. In certain embodiments, contacting the sample with a probe and/or primers (or a combination of multiple probes and primers) permits identification of both full-length and short form CEA RNA. In other embodiments, contacting the sample with a probe and/or primers uniquely identifies either full-length or short form CEA RNA. Regardless of the specific way in which the assay is conducted, it provides information regarding whether (or not) a tumor sample expresses full-length CEA RNA and/or short form CEA RNA. This information may be useful in determining whether a patient is potentially susceptible to treatment with a cancer therapeutic that immunospecifically binds to a particular target CEA protein (either long form or short form).

Without being bound by theory, it is unlikely that tumors that do not express a particular target form of CEA RNA, or which express low levels of that RNA, would be responsive to a treatment regimen based on administering a therapeutic agent that immunospecifically binds to that the CEA protein encoded by that RNA. Thus, for example, if a tumor sample from a patient does not express full-length CEA RNA, or expresses low levels of that RNA, the patient is not a good candidate for treatment with a therapeutic that immunospecifically binds to full-length CEA protein. Similarly, if a tumor sample from a patient does not express short form CEA RNA, or expresses a low level of that RNA, the patient is not a good candidate for treatment with a therapeutic that immunospecifically binds to short form CEA protein.

However, if a tumor sample from a patient does express one or both of full-length and/or short form CEA, the patient may be susceptible to treatment with a therapeutic that immunospecifically binds that target form of CEA protein. Thus, if the tumor sample expresses full-length CEA RNA, the patient may be susceptible to treatment with a therapeutic that immunospecifically binds to full-length CEA. If the tumor sample expresses short form CEA RNA but not full-length CEA RNA, the patient may be susceptible to treatment with a therapeutic that immunospecifically binds to short form CEA. Thus, in certain embodiments, following analysis of RNA expression in a tumor sample taken from a patient, the patient can be treated with the appropriate therapeutic, such as an anti-CEA therapeutic. Moreover, if the tumor sample does not express CEA RNA, the patient's therapeutic regimen can be crafted along other lines without unnecessary exposure to anti-CEA therapies.

In certain embodiments, if a tumor sample tests positive for RNA expression of a particular target CEA, one or more additional biological samples (e.g., blood, serum, sputum, feces, urine, etc.) may be obtained from the patient to evaluate protein expression for that particular target CEA. For example, these one or more additional biological samples may be obtained and contacted with an antibody that immunospecifically binds to full-length CEA or short form CEA to evaluate whether samples from patients whose tumors tested positive for RNA expression also test positive for protein expression. In certain embodiments, depending on the results of tests for protein expression in one or more biological samples, the subject may be treated with a cancer therapeutic that immunospecifically binds to the target CEA protein (e.g., immunospecifically binds to full-length CEA protein if the patient is positive for full-length CEA protein and/or short form CEA protein, and immunospecifically binds to short form CEA protein if the patient is positive for only short form CEA protein.)

Diagnostic methods for detecting RNA in a sample, such as a tumor sample, are well known in the art. By way of non-limiting example, RNA expression may be detected using RT-PCR analysis, SAGE, MPSS, microarray, direct sequencing or TaqMan quantitative analysis. Methods for evaluating protein expression in one or more biological samples are as provided elsewhere herein. Moreover, if the patient is identified as suitable for treatment with an agent that, for example, immunospecifically binds to full-length CEA, exemplary cancer therapeutics are provided herein.

(ix) Methods of Producing Proteins

The disclosure provides purified polypeptides comprising the amino acid sequence represented in SEQ ID NO: 1 (in the presence or absence of pro-sequences), or a fragment thereof comprising the following consecutive amino acid residues: NIIQNELSVD (SEQ ID NO: 11). The disclosure provides purified polypeptides comprising the amino acid sequence represented in SEQ ID NO: 1, or a fragment thereof comprising the following consecutive amino acid residues: QNIIQNELSVDH (SEQ ID NO: 13). The disclosure provides purified polypeptides comprising the amino acid sequence represented in SEQ ID NO: 1, or a fragment thereof comprising the following consecutive amino acid residues: IQNIIQNELSVDHS (SEQ ID NO: 14). In certain embodiments, the fragment comprises about 10, 15, 20, 25 or 30, 35, 40, 45, 50, 55, 60, 65, 70, 75, 80, 85, 90, 95, 100, 150, 200, 250, 300, 350, or 400 amino acids.

The disclosure provides purified polypeptides comprising the amino acid sequence represented in SEQ ID NO: 1 (in the presence or absence of pro-sequences), or a fragment thereof comprising the following consecutive amino acid residues: NIIQNKLSVD (SEQ ID NO: 12). The disclosure provides purified polypeptides comprising the amino acid sequence represented in SEQ ID NO: 1, or a fragment thereof comprising the following consecutive amino acid residues: QNIIQNKLSVDH (SEQ ID NO: 15). The disclosure provides purified polypeptides comprising the amino acid sequence represented in SEQ ID NO: 1, or a fragment thereof comprising the following consecutive amino acid residues: IQNIIQNKLSVDHS (SEQ ID NO: 16). In certain embodiments, the fragment comprises about 10, 15, 20, 25 or 30, 35, 40, 45, 50, 55, 60, 65, 70, 75, 80, 85, 90, 95, 100, 150, 200, 250, 300, 350, or 400 amino acids.

Target CEA protein or fragments can be produced in any of a number of ways. By way of example, such proteins and fragments can be produced synthetically. By way of further example, such proteins and fragments can be produced recombinantly.

Recombinant expression of a target CEA protein can be performed by constructing an expression vector containing a polynucleotide that encodes all or a portion of the desired target CEA protein. Once a polynucleotide encoding such a target CEA protein has been obtained, the vector for the production of the epitope binding molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an encoding nucleotide sequence are described herein. The target CEA proteins can be produced in many different expression systems. In one embodiment, the target CEA proteins are produced and secreted by mammalian cells. In another embodiment, the target CEA proteins are produced and secreted in human cells. In a specific embodiment, the target CEA proteins of the disclosure are produced in cells of the 293F, CHO, or NS0 cell line. In other embodiments, the target CEA proteins are produced in yeast or bacterial cells. In another embodiment, the target CEA proteins are produced using baculovirus mediated expression in, for example, SF9 cells.

Methods which are known to those skilled in the art can be used to construct expression vectors containing protein coding sequences and appropriate transcriptional and translational control signals. These methods include, for example, in vitro recombinant DNA techniques, synthetic techniques, and in vivo genetic recombination. The disclosure, thus, provides replicable vectors comprising a nucleotide sequence encoding a CEA protein molecule operably linked to a promoter.

Once the expression vector is transferred to a host cell by conventional techniques, the transfected cells are then cultured by conventional techniques to produce a CEA protein. Thus, the disclosure includes host cells containing a polynucleotide encoding a target CEA protein operably linked to a heterologous promoter.

A variety of host-expression vector systems may be utilized to express a CEA protein or portions thereof as described in U.S. Pat. No. 5,807,715. For example, mammalian cells such as Chinese hamster ovary cells (CHO), in conjunction with a vector such as the major intermediate early gene promoter element from human cytomegalovirus is an effective expression system for CEA proteins (Foecking et al., Gene, 45:101 (1986); and Cockett et al., Bio/Technology, 8:2 (1990)). In addition, a host cell strain may be chosen which modulates the expression of inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the protein of the disclosure. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0, CRL7O3O and HsS78Bst cells.

In bacterial systems, a number of expression vectors may be advantageously selected depending upon the use intended for the protein molecule being expressed. For example, when a large quantity of such a CEA protein is to be produced, for the generation of pharmaceutical or diagnostic compositions comprising a CEA protein, vectors which direct the expression of high levels of fusion protein products that are readily purified may be desirable. Such vectors include, but are not limited to, the E. coli expression vector pUR278 (Ruther et al., EMBO, 12:1791 (1983)), in which the coding sequence may be ligated individually into the vector in frame with the lac Z coding region so that a fusion protein is produced; pIN vectors (Inouye & Inouye, 1985, Nucleic Acids Res. 13:3101-3109 (1985); Van Heeke & Schuster, 1989, J. Biol. Chem., 24:5503-5509 (1989)); and the like. pGEX vectors may also be used to express foreign polypeptides as fusion proteins with glutathione-S-transferase (GST). In general, such fusion proteins are soluble and can easily be purified from lysed cells by adsorption and binding to glutathione-agarose affinity matrix followed by elution in the presence of free glutathione. The pGEX vectors are designed to introduce a thrombin and/or factor Xa protease cleavage sites into the expressed polypeptide so that the cloned target gene product can be released from the GST moiety.

In an insect system, Autographa californica nuclear polyhedrosis virus (AcNPV) is used as a vector to express foreign genes. The virus grows in Spodoptera frugiperda cells. The protein coding sequence may be cloned individually into non-essential regions (for example, the polyhedrin gene) of the virus and placed under control of an AcNPV promoter (for example, the polyhedrin promoter).

In mammalian host cells, a number of virus based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion into a non-essential region of the viral genome (e.g., region E1 or E3) will result in a recombinant virus that is viable and capable of expressing the antibody molecule in infected hosts (e.g., see, Logan & Shenk, Proc. Natl. Acad. Sci. USA, 81:355-359 (1984)). Specific initiation signals may also be required for efficient translation. These signals include the ATG initiation codon and adjacent sequences. Furthermore, the initiation codon should generally be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol., 153:51-544 (1987)).

(x) Methods of Making Antibodies

This disclosure also provides monoclonal anti-CEA antibodies. A monoclonal antibody can be obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are often synthesized by the hybridoma culture, uncontaminated by other immunoglobulins. Monoclonal antibodies may also be produced in transfected cells, such as CHO cells and NS0 cells. The modifier “monoclonal” indicates the character of the antibody as being obtained from a substantially homogeneous population of antibodies and does not require production of the antibody by any particular method. For example, the monoclonal antibodies to be used in accordance with the present disclosure may be made by the hybridoma method first described by Kohler et al., Nature 1975; 256:495, or may be made by recombinant DNA methods (see, e.g., U.S. Pat. Nos. 4,816,567 and 6,331,415). The “monoclonal antibodies” may also be isolated from phage antibody libraries using the techniques described in Clackson et al., Nature 1991; 352:624-628 and Marks et al., J. Mol. Biol. 1991; 222:581-597, for example.

General methods for the immunization of animals (in this case with CEA), isolation of antibody producing cells, fusion of such cells with immortal cells (e.g., myeloma cells) to generate hybridomas secreting monoclonal antibodies, screening of hybridoma supernatants for reactivity of secreted monoclonal antibodies with a desired antigen (in this case the immunogen or a molecule containing the immunogen), the preparation of quantities of such antibodies in hybridoma supernatants or ascites fluids, and for the purification and storage of such monoclonal antibodies, can be found in numerous publications. These include: Coligan et al., eds. Current Protocols In Immunology, John Wiley & Sons, New York, 1992; Harlow and Lane, Antibodies, A Laboratory Manual, Cold Spring Harbor Laboratory, New York, 1988; Liddell and Cryer, A Practical Guide To Monoclonal Antibodies, John Wiley & Sons, Chichester, West Sussex, England, 1991; Montz et al., Cellular Immunol. 127:337-351, 1990; Wurzner et al., Complement Inflamm. 8:328-340, 1991; and Mollnes et al., Scand. J. Immunol. 28:307-312, 1988.

Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Alternatively, when the above-described methods are used for producing polyclonal antibodies, then following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, in order to monitor the success of immunization, the antibody levels with respect to the antigen in serum will be monitored using standard techniques such as ELISA, RIA and the like.

Furthermore, the anti-CEA antibodies can also be produced via CEA immunization of a transgenic mouse lacking the genes encoding mouse CEA in their genomes (a CEA knock-out mouse).

Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. In certain embodiments, the fusion is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96/27011; Brennan et al., Science 229:81 (1985); Shalaby et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991). Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. A strategy for making bispecific antibody fragments by the use of single-chain Fv (scFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

In order to produce the chimeric antibodies, the portions derived from two different species (e.g., human constant region and murine variable or binding region) can be joined together chemically by conventional techniques or can be prepared as single contiguous proteins using genetic engineering techniques. The DNA molecules encoding the proteins of both the light chain and heavy chain portions of the chimeric antibody can be expressed as contiguous proteins. The method of making chimeric antibodies is disclosed in U.S. Pat. No. 5,677,427; U.S. Pat. No. 6,120,767; and U.S. Pat. No. 6,329,508, each of which is incorporated by reference in its entirety.

Fully human antibodies against CEA may be produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety).

Human antibodies against CEA can also be produced from non-human transgenic animals having transgenes encoding at least a segment of the human immunoglobulin locus. The production and properties of animals having these properties are described in detail by, see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety.

Various recombinant antibody library technologies may also be utilized to produce fully human antibodies. For example, one approach is to screen a DNA library from human B cells according to the general protocol outlined by Huse et al., Science 246:1275-1281 (1989). Antibodies binding CEA or a fragment thereof are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047; U.S. Pat. No. 5,969,108, (each of which is incorporated by reference in its entirety). In these methods, libraries of phage are produced in which members display different antibodies on their outer surfaces. Antibodies are usually displayed as Fv or Fab fragments. Phage displaying antibodies with a desired specificity are selected by affinity enrichment to CEA or a fragment thereof. Additional approaches may be used with this application (U.S. Patent Application Nos. 20040072164 and 20040175736, each of which is incorporated by reference in its entirety).

Eukaryotic ribosome can also be used as means to display a library of antibodies and isolate the binding human antibodies by screening against the target antigen, such as CEA, as described in Coia G, et al., J. Immunol. Methods 1: 254 (1-2):191-7 (2001); Hanes J. et al., Nat. Biotechnol. 18(12):1287-92 (2000); Proc. Natl. Acad. Sci. U.S.A. 95(24):14130-5 (1998); Proc. Natl. Acad. Sci. U.S.A. 94(10):4937-42 (1997), each which is incorporated by reference in its entirety.

The yeast system is also suitable for screening mammalian cell-surface or secreted proteins, such as antibodies. Antibody libraries may be displayed on the surface of yeast cells for the purpose of obtaining the human antibodies against a target antigen. This approach is described by Yeung, et al., Biotechnol. Prog. 18(2):212-20 (2002); Boeder, E. T., et al., Nat. Biotechnol. 15(6):553-7 (1997), each of which is herein incorporated by reference in its entirety. Alternatively, human antibody libraries may be expressed intracellularly and screened via the yeast two-hybrid system (WO0200729A2, which is incorporated by reference in its entirety).

Recombinant DNA techniques can be used to produce the recombinant anti-CEA antibodies, as well as the chimeric or humanized anti-CEA antibodies or any other anti-CEA genetically-altered antibodies and the fragments or conjugate thereof in any expression systems including both prokaryotic and eukaryotic expression systems, such as bacteria, yeast, insect cells, plant cells, mammalian cells (for example, NS0 cells).

Once produced, the whole antibodies, their dimers, individual light and heavy chains, or other immunoglobulin forms of the present application can be purified according to standard procedures of the art, including ammonium sulfate precipitation, affinity columns, column chromatography, gel electrophoresis and the like (see, generally, Scopes, R., Protein Purification (Springer-Verlag, N.Y., 1982)). In certain embodiments, substantially pure immunoglobulins, such as for example, at least about 90 to 95% homogeneity, in certain embodiments 98 to 99% or more homogeneity, may be used for pharmaceutical purposes. Once purified, partially or to homogeneity as desired, the polypeptides may then be used therapeutically (including extracorporeally) or in developing and performing assay procedures, immunofluorescent stainings, and the like. (See, generally, Immunological Methods, Vols. I and II (Lefkovits and Pernis, eds., Academic Press, NY, 1979 and 1981).

### Exemplary Embodiments

1. A method of detecting recurrence of a carcinoembryonic antigen (CEA) expressing cancer comprising

obtaining a sample from a subject previously diagnosed with and treated for a carcinoembryonic antigen (CEA) expressing cancer;

detecting in said sample a concentration of full-length CEA protein using an antibody, an antigen binding fragment or an immunoglobulin-like molecule that immunospecifically binds to full-length CEA protein but does not immunospecifically bind to short form CEA protein, thereby detecting the concentration of full-length CEA protein without detecting the concentration of short form CEA protein in said sample,

wherein detecting a concentration of full-length CEA protein in said sample above a concentration observed after treatment indicates recurrence of said CEA expressing cancer.
2. A method of detecting recurrence of a carcinoembryonic antigen (CEA) expressing cancer comprising

obtaining a first sample from a subject having a carcinoembryonic antigen (CEA) expressing cancer, wherein said first sample is obtained prior to treatment;

detecting in said first sample a pre-treatment concentration of full-length CEA protein using an antibody, an antigen binding fragment or an immunoglobulin-like molecule that immunospecifically binds to full-length CEA protein but does not immunospecifically bind to short form CEA protein, thereby detecting the concentration of full-length CEA protein without detecting the concentration of short form CEA protein in said first sample;

obtaining a second sample from said subject, and detecting in said second sample a concentration of full-length CEA protein using said antibody, antigen binding fragment or immunoglobulin-like molecule, thereby detecting the concentration of full-length CEA protein without detecting the concentration of short form CEA protein in said second sample;

obtaining one or more further samples from said subject at a time later than that for obtaining said second sample, and detecting in said one or more further samples a concentration of full-length CEA protein using said antibody, antigen binding fragment or immunoglobulin-like molecule, thereby detecting the concentration of full-length CEA protein without detecting the concentration of short form CEA protein in said one or more further samples,

wherein detecting a concentration of full-length CEA protein in said one or more further samples above the concentration of full-length CEA protein observed in said second sample indicates recurrence of said CEA expressing cancer.
3. A method of determining susceptibility to anti-carcinoembryonic antigen (CEA) cancer therapeutic comprising

detecting a concentration of full-length CEA protein in a sample from a subject using an antibody, an antigen binding fragment or an immunoglobulin-like molecule that immunospecifically binds to full-length CEA protein but does not immunospecifically bind to short form CEA protein, thereby detecting the concentration of full-length CEA protein without detecting the concentration of short form CEA protein in said sample and

comparing said concentration of full-length CEA protein to a standard range reflecting full-length CEA protein concentration in samples from healthy subjects;

wherein detecting a concentration of full-length CEA protein above said standard range indicates susceptibility to anti-CEA cancer therapy.
4. A method of monitoring anti-carcinoembryonic antigen (CEA) cancer therapy comprising

detecting a concentration of full-length CEA protein in a sample from a subject undergoing treatment for a CEA expressing cancer using an antibody, an antigen binding fragment or an immunoglobulin-like molecule that immunospecifically binds to full-length CEA protein but does not immunospecifically bind to short form CEA protein, thereby detecting the concentration of full-length CEA protein without detecting the concentration of short form CEA protein in said sample and

comparing said concentration of full-length CEA protein to a concentration of full-length CEA protein in a sample from said same subject, which sample was obtained prior to said treatment or at an earlier time point during said treatment;

wherein a decrease in full-length CEA concentration in a sample obtained at a later point during treatment versus that obtained prior to treatment or at an earlier time point during said treatment indicates effectiveness of said treatment, thereby monitoring said anti-CEA cancer therapy.
5. A method of treating a subject having a carcinoembryonic antigen (CEA) expressing cancer comprising

obtaining a sample from a subject prior to treatment for a carcinoembryonic antigen (CEA) expressing cancer;

detecting in said sample a concentration of full-length CEA protein using an antibody, an antigen binding fragment or an immunoglobulin-like molecule that immunospecifically binds to full-length CEA protein but does not immunospecifically bind to short form CEA protein, thereby detecting the concentration of full-length CEA protein without detecting the concentration of short form CEA protein in said sample;

comparing said concentration of full-length CEA protein to a standard range reflecting full-length CEA protein concentration in samples from healthy subjects, wherein detecting the concentration of full-length CEA protein above said standard range indicates susceptibility to anti-CEA cancer therapy;

treating said subject with an anti-CEA cancer therapeutic if said subject is determined to be susceptible to anti-CEA cancer therapy;

optionally detecting a concentration of full-length CEA protein in a post-treatment sample from said subject using an antibody, an antigen binding fragment or an immunoglobulin-like molecule that immunospecifically binds to full-length CEA protein but does not immunospecifically bind to a short form CEA protein, thereby detecting the concentration of full-length CEA protein without detecting the concentration of short form CEA protein in said post-treatment sample; and

comparing said concentration of full-length CEA protein in said post-treatment sample to said concentration in the sample obtained prior to treatment,

wherein a decrease in full-length CEA protein concentration in said post-treatment sample relative to said pre-treatment sample indicates the effectiveness of said anti-CEA cancer therapeutic in said method of treating said subject.
6. A method of determining susceptibility to a cancer therapeutic that immunospecifically binds to carcinoembryonic antigen (CEA) protein comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of said target CEA protein in a sample from said subject using an antibody, an antigen binding fragment or an immunoglobulin-like molecule that immunospecifically binds to an epitope on said target CEA protein that is the same or substantially the same as the epitope that said cancer therapeutic immunospecifically binds, thereby detecting the concentration of the target CEA protein without detecting the concentration of non-target forms of CEA protein in said sample; and

comparing said concentration of said target CEA protein to a standard range reflecting target CEA protein concentration in samples from healthy subjects; wherein detecting a concentration of said target CEA protein in said sample above said standard range indicates susceptibility to said cancer therapeutic.

7. A method of monitoring treatment comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of a said target CEA protein in a sample from said subject, which subject is undergoing treatment for a CEA expressing cancer, using an antibody, an antigen binding fragment or an immunoglobulin-like molecule that immunospecifically binds to an epitope on said target CEA protein that is the same or substantially the same as the epitope that said cancer therapeutic immunospecifically binds, thereby detecting the concentration of a target CEA protein without detecting the concentration of other non-target forms of CEA protein in said sample; and

comparing said concentration of target CEA protein to a concentration of target CEA protein in an earlier sample from said same subject, which earlier sample was obtained prior to treatment with said cancer therapeutic or at an earlier time point during treatment with said cancer therapeutic;

wherein a decrease in target CEA concentration in a sample obtained at a later point during treatment with said cancer therapeutic versus that obtained prior to treatment or at an earlier time point during treatment with said cancer therapeutic indicates effectiveness of said cancer therapeutic, thereby monitoring said treatment.
8. A method of treating a subject having a carcinoembryonic antigen (CEA) expressing cancer comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of said target CEA protein in a sample from said subject using an antibody, an antigen binding fragment or an immunoglobulin-like molecule that immunospecifically binds to an epitope on said target CEA protein that is the same or substantially the same as the epitope that said therapeutic immunospecifically binds, thereby detecting the concentration of said target CEA protein without detecting the concentration of other non-target forms of CEA protein in said sample;

comparing said concentration of said target CEA protein to a standard range reflecting target CEA protein concentration in samples from healthy subjects;

wherein detecting a concentration of said target CEA protein above said standard range indicates susceptibility to a cancer therapeutic that immunospecifically binds to target CEA protein;

treating said subject with said cancer therapeutic that immunospecifically binds to target CEA protein if said subject is determined to be susceptible to said cancer therapeutic;

optionally detecting, in a post-treatment sample from said subject undergoing treatment with said cancer therapeutic, a concentration of target CEA protein using an antibody, an antigen binding fragment or an immunoglobulin-like molecule that immunospecifically binds to an epitope on target CEA that is the same or substantially the same as the epitope that said cancer therapeutic immunospecifically binds, thereby detecting the concentration of said target CEA protein without detecting the concentration of other non-target forms of CEA protein in said sample; and

comparing said concentration of target CEA protein to a concentration of target CEA protein in a sample from said same subject, which sample was obtained prior to treatment with said cancer therapeutic or at an earlier time point during said treatment;

wherein a decrease in target CEA concentration in a sample obtained at a later point during treatment with said cancer therapeutic versus that obtained prior to or at an earlier time point during said treatment indicates effectiveness of said treatment of said subject.
9. A method comprising

detecting a concentration of full-length CEA protein and a concentration of short form CEA protein in a sample from a subject and

determining a ratio of full-length CEA protein concentration to short form CEA protein concentration.

10. The method of embodiment 9, further comprising

comparing said ratio to a standard reflecting the standard ratio of full-length CEA protein concentration to short form CEA protein concentration in samples from healthy subjects;

wherein a ratio higher or lower than the standard ratio is indicative of presence of a CEA-expressing cancer.
11. The method of embodiment 9 or 10, wherein detecting a concentration of full-length CEA protein comprises contacting a sample with an antibody, an antigen binding fragment or an immunoglobulin-like molecule that immunospecifically binds to full-length CEA protein but does not immunospecifically bind to short form CEA protein.
12. The method of any of embodiments 9-11, wherein detecting a concentration of full-length CEA protein comprises contacting a sample with an antibody, an antigen binding fragment or an immunoglobulin-like molecule that immunospecifically binds to full-length CEA protein but does not immunospecifically bind to short form CEA protein, and detecting a concentration of short form CEA protein comprises contacting a sample with an antibody, an antigen binding fragment or an immunoglobulin-like molecule that immunospecifically binds to short form CEA protein but does not immunospecifically bind to full-length CEA protein.
13. The method of any of embodiments 1-4 or 6-7, further comprising treating said subject with an anti-CEA cancer therapeutic.
14. The method of any of embodiments 5, 8, or 13, wherein said cancer therapeutic immunospecifically binds to an epitope on CEA protein that is the same or substantially the same as that immunospecifically bound by said antibody, antigen binding fragment or immunoglobulin-like molecule used in said detecting steps.
15. The method of any of embodiments 1-14, wherein said subject is a human.
16. The method of any of embodiments 3-8, wherein said cancer therapeutic comprises a protein therapeutic.
17. The method of embodiment 16, wherein said protein therapeutic is an antibody or antigen binding fragment.
18. The method of embodiment 17, wherein said protein therapeutic is a monoclonal antibody.
19. The method of embodiment 18, wherein said monoclonal antibody is a chimeric antibody, a humanized antibody, or a fully human antibody.
20. The method of any of embodiment 17-19, wherein said protein therapeutic immunospecifically binds to a protein comprising the amino acid sequence of SEQ ID NO: 2.
21. The method of embodiment 20, wherein said protein therapeutic immunospecifically binds to a protein comprising the amino acid sequence of SEQ ID NO:2, but which protein therapeutic does not immunospecifically bind to a protein comprising the amino acid sequence of SEQ ID NO: 1.
22. The method of embodiment 17, wherein said protein therapeutic comprises an antigen binding domain of antibody A5B7.
23. The method of embodiment 17, wherein said protein therapeutic is a bispecific antibody.
24. The method of embodiment 23, wherein said bispecific antibody comprises a CEA binding portion and a CD3 binding portion.
25. The method of any of embodiments 1-24, wherein said sample is chosen from whole blood, serum, plasma, saliva, urine, feces, seminal plasma, sweat, amniotic fluid, sputum, breast milk, bile, tissue homogenate, and ascites.
26. The method of any of embodiments 1-24, wherein said sample is a tumor tissue sample.
27. The method of any of embodiments 1-26, wherein said CEA-expressing cancer is chosen from colon cancer, rectal cancer, pancreatic cancer, esophageal cancer, gastroesophageal cancer, stomach cancer, lung cancer and breast cancer.
28. The method of any of embodiments 1-8, wherein said detecting step comprises

contacting said sample with said antibody, antigen binding fragment or immunoglobulin-like molecule and

detecting the concentration of full-length CEA protein by immunohistochemistry.

29. The method of embodiment 28, wherein said sample is contacted with said antibody, and wherein said antibody is a monoclonal antibody.
30. The method of embodiment 29, wherein said monoclonal antibody is a chimeric antibody, a humanized antibody, or a fully human antibody.
31. The method of any of embodiments 1-8, wherein said detecting step comprises

contacting said sample with said antibody, antigen binding fragment or immunoglobulin-like molecule, which antibody, antigen binding fragment or immunoglobulin-like molecule binds to a protein comprising the amino acid sequence of SEQ ID NO:2 and

detecting the concentration of full-length CEA protein by immunohistochemistry.

32. The method of any of embodiments 1-8 or 28-31, wherein said detecting step comprises

contacting said sample with an antibody or antigen binding fragment comprising the antigen binding domain of antibody A5B7, with the proviso that the antibody is not A5B7.

33. The method of any of embodiments 1-8 or 28-31, wherein said detecting step comprises

contacting said sample with an antibody or antigen binding fragment comprising an antigen binding domain that binds the same or substantially the same epitope as A5B7, with the proviso that the antibody is not A5B7.

34. The method of any of embodiments 1-8 or 28-31, wherein said detecting step comprises

contacting said sample with an antibody or antigen binding fragment comprising an antigen binding domain that binds the same or substantially the same epitope as A5B7, with the proviso that the antibody is not A5B7 or a bispecific antibody.

35. A method of detecting expression of short form carcinoembryonic antigen (CEA) RNA in a biological sample comprising

providing one or both of a nucleic acid probe or nucleic acid primers that hybridize to a CEA nucleotide sequence, and which specifically identify expression of short form CEA by (i) hybridizing specifically to a short form CEA nucleotide sequence but not to a full-length CEA nucleotide sequence or (ii) hybridizing specifically to both short form CEA nucleotide sequence and full-length CEA nucleotide sequence in a manner that distinguishes expression of short form CEA from expression of full-length CEA;

providing RNA from a biological sample; and

detecting expression of short form CEA RNA in said biological sample using said nucleic acid probe or nucleic acid primers.

36. A method of detecting expression of short form carcinoembryonic antigen (CEA) protein in a biological sample comprising

providing an antibody, antigen binding fragment or immunoglobulin-like molecule that immunospecifically binds to short form CEA protein but does not immunospecifically bind to full-length CEA protein;

providing a biological sample; and

detecting expression of short form CEA protein in said biological sample using said antibody.

37. The method of embodiment 35 or 36, wherein said biological sample is a tumor tissue sample.
38. The method of embodiment 36, wherein said biological sample is chosen from whole blood, serum, plasma, saliva, urine, feces, seminal plasma, sweat, amniotic fluid, tissue homogenate, sputum, breast milk, bile, and ascites.
39. The method of embodiment 36, comprising providing an antibody, wherein said antibody is a monoclonal antibody.
40. The method of embodiment 39, wherein said monoclonal antibody is a chimeric antibody, a humanized antibody, or a fully human antibody.
41. The method of embodiment 36, wherein said antibody binds to a protein comprising the amino acid sequence of SEQ ID NO:1.
42. A method of generating antibodies immunospecific for full-length carcinoembryonic antigen (CEA) protein comprising

providing a portion of full-length CEA protein that is not present in short form CEA protein and

using said portion of full-length CEA protein as an antigen for generating said antibodies.

43. The method of embodiment 42, wherein said antibodies are monoclonal antibodies.
44. A purified polypeptide comprising the amino acid sequence represented in SEQ ID NO: 1, or a fragment thereof comprising the following consecutive amino acid residues: NIIQNELSVD (SEQ ID NO: 11) or NIIQNKLSVD (SEQ ID NO: 12).
45. A method of identifying patients that may be susceptible to a cancer therapeutic that immunospecifically binds to a target carcinoembryonic antigen (CEA) protein comprising

obtaining a sample from a patient;

detecting in the sample expression of a target CEA RNA to distinguish RNA expression of full-length CEA from RNA expression of short form CEA;

wherein, if the tumor sample from the patient expresses said target CEA RNA, the patient may be susceptible to treatment with a cancer therapeutic that immunospecifically binds to that target CEA protein, and wherein, if the tumor sample from the patient does not express said target CEA RNA, the patient will not be susceptible to treatment with a cancer therapeutic that immunospecifically binds to that target CEA protein.
46. The method of embodiment 45, wherein detecting target CEA RNA expression comprises contacting the sample with probe and/or primers to evaluate expression of full-length CEA RNA.
47. The method of embodiment 45, wherein detecting target CEA RNA expression comprises contacting the sample with probe and/or primers to evaluate expression of short form CEA RNA.
48. The method of embodiment 45, wherein detecting target CEA RNA expression comprises contacting the sample with probe and/or primers to evaluate expression of both full-length CEA RNA and short form CEA RNA.
49. The method of any of embodiments 45-48, wherein the method further comprises taking one or more additional biological samples from said patient, and assaying the one or more biological samples for expression of the target CEA protein.
50. The method of any of embodiments 45-49, wherein the method further comprises treating said subject with a cancer therapeutic.
51. The method of any of embodiments 1-8, 11-34 and 36-41, wherein the antibody, an antigen binding fragment or an immunoglobulin-like molecule comprises an amino acid sequence chosen from the amino acid sequences of SEQ ID NOs: 28-44 and 46-51.
52. The method of any of embodiments 3, 5-8, 13-34, and 45-50 wherein the anti-CEA therapeutic comprises an amino acid sequence chosen from the amino acid sequences of SEQ ID NOs: 28-44 and 46-51.
53. The method of embodiment 51, wherein the antibody, an antigen binding fragment or an immunoglobulin-like molecule comprises the amino acid sequence of SEQ ID NO: 48.
54. The method of embodiment 51, wherein the antibody, an antigen binding fragment or an immunoglobulin-like molecule comprises the amino acid sequence of SEQ ID NO: 49.
55. The method of embodiment 51, wherein the antibody, an antigen binding fragment or an immunoglobulin-like molecule comprises the amino acid sequence of SEQ ID NO: 46.
56. The method of embodiment 51, wherein the antibody, an antigen binding fragment or an immunoglobulin-like molecule comprises the amino acid sequence of SEQ ID NO: 51.
57. The method of embodiment 52, wherein the anti-CEA therapeutic comprises the amino acid sequence of SEQ ID NO: 48.
58. The method of embodiment 52, wherein anti-CEA therapeutic comprises the amino acid sequence of SEQ ID NO: 49.
59. The method of embodiment 52, wherein the anti-CEA therapeutic comprises the amino acid sequence of SEQ ID NO: 46.

59. The method of embodiment 60, wherein the anti-CEA therapeutic comprises the amino acid sequence of SEQ ID NO: 51.

60. A method of detecting recurrence of a carcinoembryonic antigen (CEA) expressing cancer comprising

obtaining a sample from a subject previously diagnosed with and treated for a carcinoembryonic antigen (CEA) expressing cancer;

detecting in said sample a concentration of RNA encoding full-length CEA protein, wherein detecting a concentration of full-length CEA RNA in said sample above a concentration observed after treatment indicates recurrence of said CEA expressing cancer.

61. A method of detecting recurrence of a carcinoembryonic antigen (CEA) expressing cancer comprising

obtaining a first sample from a subject having a carcinoembryonic antigen (CEA) expressing cancer, wherein said first sample is obtained prior to treatment;

detecting in said first sample a pre-treatment concentration of RNA encoding full-length CEA;

obtaining a second sample from said subject, and detecting in said second sample a concentration of RNA encoding full-length CEA protein

obtaining one or more further samples from said subject at a time later than that for obtaining said second sample, and detecting in said one or more further samples a concentration of RNA encoding full-length CEA protein,

wherein detecting a concentration of RNA encoding full-length CEA protein in said one or more further samples above the concentration of RNA encoding full-length CEA protein observed in said second sample indicates recurrence of said CEA expressing cancer.
62. A method of determining susceptibility to anti-carcinoembryonic antigen (CEA) cancer therapeutic comprising

detecting a concentration of RNA encoding full-length CEA protein in a sample from a subject and

comparing said concentration of RNA encoding full-length CEA protein to a standard range reflecting the concentration of RNA encoding full-length CEA protein in samples from healthy subjects;

wherein detecting a concentration of RNA encoding full-length CEA protein above said standard range indicates susceptibility to anti-CEA cancer therapy.
63. A method of monitoring anti-carcinoembryonic antigen (CEA) cancer therapy comprising

detecting a concentration of RNA encoding full-length CEA protein in a sample from a subject undergoing treatment for a CEA expressing and

comparing said concentration of RNA encoding full-length CEA protein to a concentration of RNA encoding full-length CEA protein in a sample from said same subject, which sample was obtained prior to said treatment or at an earlier time point during said treatment;

wherein a decrease in RNA encoding full-length CEA concentration in a sample obtained at a later point during treatment versus that obtained prior to treatment or at an earlier time point during said treatment indicates effectiveness of said treatment, thereby monitoring said anti-CEA cancer therapy.
64. A method of treating a subject having a carcinoembryonic antigen (CEA) expressing cancer comprising

obtaining a sample from a subject prior to treatment for a carcinoembryonic antigen (CEA) expressing cancer;

detecting in said sample a concentration of RNA encoding full-length CEA;

comparing said concentration of RNA encoding full-length CEA protein to a standard range reflecting the concentration of RNA encoding full-length CEA protein concentration in samples from healthy subjects,

wherein detecting the concentration of RNA encoding full-length CEA protein above said standard range indicates susceptibility to anti-CEA cancer therapy;

treating said subject with an anti-CEA cancer therapeutic if said subject is determined to be susceptible to anti-CEA cancer therapy;

optionally detecting a concentration of RNA encoding full-length CEA protein in a post-treatment sample from said subject; and

comparing said concentration of RNA encoding full-length CEA protein in said post-treatment sample to said concentration in the sample obtained prior to treatment, wherein a decrease in RNA encoding full-length CEA protein concentration in said post-treatment sample relative to said pre-treatment sample indicates the effectiveness of said anti-CEA cancer therapeutic in said method of treating said subject.

65. A method of determining susceptibility to a cancer therapeutic that immunospecifically binds to carcinoembryonic antigen (CEA) protein comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of RNA encoding said target CEA protein in a sample from said subject without detecting the concentration of RNA encoding non-target forms of CEA protein; and

comparing said concentration of said RNA encoding said target CEA protein to a standard range reflecting the concentration of RNA encoding said target CEA protein in samples from healthy subjects;

wherein detecting a concentration of said RNA encoding said target CEA protein in said sample above said standard range indicates susceptibility to said cancer therapeutic.
66. A method of monitoring treatment comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of a RNA encoding said target CEA protein in a sample from said subject, which subject is undergoing treatment for a CEA expressing cancer, without detecting the concentration of non-target forms of CEA protein in said sample; and

comparing said concentration of RNA encoding said target CEA protein to a concentration of RNA encoding said target CEA protein in an earlier sample from said same subject, which earlier sample was obtained prior to treatment with said cancer therapeutic or at an earlier time point during treatment with said cancer therapeutic;

wherein a decrease in RNA encoding said target CEA concentration in a sample obtained at a later point during treatment with said cancer therapeutic versus that obtained prior to treatment or at an earlier time point during treatment with said cancer therapeutic indicates effectiveness of said cancer therapeutic, thereby monitoring said treatment.
67. A method of treating a subject having a carcinoembryonic antigen (CEA) expressing cancer comprising

selecting a cancer therapeutic that will be used in the treatment of a subject with a CEA-expressing cancer, which cancer therapeutic immunospecifically binds to one form of CEA protein but does not immunospecifically bind to a second form of CEA protein, which one form of CEA protein is referred to as target CEA protein;

detecting a concentration of RNA encoding said target CEA protein in a sample from said subject using primers and/or probe to detect RNA encoding an epitope on said target CEA protein that is the same or substantially the same as the epitope that said therapeutic immunospecifically binds, thereby detecting the concentration of RNA encoding said RNA target CEA protein without detecting the concentration of RNA encoding non-target forms of CEA protein in said sample;

comparing said concentration of RNA encoding said target CEA protein to a standard range reflecting target CEA protein concentration in samples from healthy subjects;

wherein detecting a concentration of said target CEA protein above said standard range indicates susceptibility to a cancer therapeutic that immunospecifically binds to target CEA protein;

treating said subject with said cancer therapeutic that immunospecifically binds to target CEA protein if said subject is determined to be susceptible to said cancer therapeutic;

optionally detecting, in a post-treatment sample from said subject undergoing treatment with said cancer therapeutic, a concentration of RNA encoding target CEA protein, thereby detecting the concentration of said target CEA protein without detecting the concentration of RNA encoding non-target forms of CEA protein in said sample; and

comparing said concentration of RNA encoding said target CEA protein to a concentration of RNA encoding said target CEA protein in a sample from said same subject, which sample was obtained prior to treatment with said cancer therapeutic or at an earlier time point during said treatment;

wherein a decrease in RNA encoding said target CEA concentration in a sample obtained at a later point during treatment with said cancer therapeutic versus that obtained prior to or at an earlier time point during said treatment indicates effectiveness of said treatment of said subject.
68. A method comprising

detecting a concentration of RNA encoding full-length CEA protein and a concentration of RNA encoding short form CEA protein in a sample from a subject and

determining a ratio of concentration of RNA encoding full-length CEA protein to concentration of RNA encoding short form CEA protein concentration.

69. The method of embodiment 68, further comprising

comparing said ratio to a standard reflecting the standard ratio of concentration of RNA encoding full-length CEA protein to concentration of RNA encoding short form CEA protein in samples from healthy subjects;

wherein a ratio higher or lower than the standard ratio is indicative of presence of a CEA-expressing cancer.
11. The method of embodiment 68 or 69, wherein detecting a concentration of RNA encoding full-length CEA protein comprises contacting a sample with primers and or probes that hybridize to RNA encoding full-length CEA protein but do not hybridize to short form CEA protein.
71. The method of any of embodiments 68-70, wherein detecting a concentration of RNA encoding full-length CEA protein comprises contacting a sample with primers and or probes that hybridize to RNA encoding full-length CEA protein but do not hybridize to short form CEA protein, and detecting a concentration of RNA encoding short form CEA protein comprises contacting a sample with primers and or probes that hybridize to RNA encoding short form CEA protein but do not hybridize to full-length CEA protein.
72. The method of any of embodiments 60-63 or 65-66, further comprising treating said subject with an anti-CEA cancer therapeutic.
73. The method of any of embodiments 64, 67, or 72, wherein said cancer therapeutic immunospecifically binds to an epitope on CEA protein that is the same or substantially the same as that immunospecifically bound by said antibody, antigen binding fragment or immunoglobulin-like molecule used in said detecting steps.
74. The method of any of embodiments 60-73, wherein said subject is a human.
75. The method of any of embodiments 63-67, wherein said cancer therapeutic comprises a protein therapeutic.
76. The method of embodiment 75, wherein said protein therapeutic is an antibody or antigen binding fragment.
77. The method of embodiment 76, wherein said protein therapeutic is a monoclonal antibody.
78. The method of embodiment 77, wherein said monoclonal antibody is a chimeric antibody, a humanized antibody, or a fully human antibody.
79. The method of any of embodiment 76-78, wherein said protein therapeutic immunospecifically binds to a protein comprising the amino acid sequence of SEQ ID NO: 2.
80. The method of embodiment 79, wherein said protein therapeutic immunospecifically binds to a protein comprising the amino acid sequence of SEQ ID NO:2, but which protein therapeutic does not immunospecifically bind to a protein comprising the amino acid sequence of SEQ ID NO: 1.
81. The method of embodiment 76, wherein said protein therapeutic comprises an antigen binding domain of antibody A5B7.
82. The method of embodiment 76, wherein said protein therapeutic is a bispecific antibody.
83. The method of embodiment 82, wherein said bispecific antibody comprises a CEA binding portion and a CD3 binding portion.
84. The method of any of embodiments 60-83, wherein said sample is chosen from whole blood, serum, plasma, saliva, urine, feces, seminal plasma, sweat, amniotic fluid, sputum, breast milk, bile, tissue homogenate, and ascites.
85. The method of any of embodiments 60-83, wherein said sample is a tumor tissue sample.
86. The method of any of embodiments 60-85, wherein said CEA-expressing cancer is chosen from colon cancer, rectal cancer, pancreatic cancer, esophageal cancer, gastroesophageal cancer, stomach cancer, lung cancer and breast cancer.
87. The method of any of embodiments 60-67, wherein said detecting step comprises the method of embodiment 35 or 45.
88. The method of embodiment 87, wherein said sample is contacted with the probe/and or primers of embodiment 46.
89. The method of embodiment 87, wherein said sample is contacted with the probe/and or primers of embodiment 47 and optionally, the probe and/or primers of embodiment 46.
90. The method of any of embodiments 60-67, wherein said detecting step comprises

contacting said sample with primers and/or probes that hybridize to an RNA encoding a protein comprising the amino acid sequence of SEQ ID NO:2.

91. The method of any of embodiments 60-67 or 87-90, wherein said detecting step comprises contacting said sample with contacting said sample with primers and/or probes that hybridize to an RNA encoding a protein comprising an epitope bound by the antigen binding domain of antibody A5B7.
92. The method of any of embodiments 60-67 or 87-90, wherein said detecting step comprises

contacting said sample with primers and/or probes that hybridize to an RNA encoding a protein comprising the same or substantially the same epitope as bound by A5B7.

93. The method of any of embodiments 60-67 or 87-90, wherein said detecting step comprises

contacting said sample with primers and/or probes that hybridize to an RNA encoding a protein that does not comprise the same or substantially the same epitope as bound by A5B7.

94. A method of detecting expression of short form carcinoembryonic antigen (CEA) protein in a biological sample comprising

providing primer and or probes that hybridize to RNA encoding short form CEA protein but does to RNA encoding full-length CEA protein;

providing a biological sample; and

detecting expression of RNA encoding short form CEA protein in said biological sample using said primers and or probes.

95. The method of embodiment 94, wherein said biological sample is a tumor tissue sample.
96. The method of embodiment 94, wherein said biological sample is chosen from whole blood, serum, plasma, saliva, urine, feces, seminal plasma, sweat, amniotic fluid, tissue homogenate, sputum, breast milk, bile, and ascites.
97. The method of embodiment 94, comprising providing primers.
98. The method of embodiment 94, comprising providing primers and probe.
99. The method of embodiment 94, wherein said primers and/or probe bind to an RNA encoding a protein comprising the amino acid sequence of SEQ ID NO:1.
100. The method of any of embodiments 60-67, 70-93 and 94-99, wherein the antibody, an antigen binding fragment or an immunoglobulin-like molecule comprises an amino acid sequence chosen from the amino acid sequences of SEQ ID NOs: 28-44 and 46-51.
101. The method of any of embodiments 62, 64-67 and 72-93, wherein the anti-CEA therapeutic comprises an amino acid sequence chosen from the amino acid sequences of SEQ ID NOs: 28-44 and 46-51.
102. The method of embodiment 100, wherein the antibody, an antigen binding fragment or an immunoglobulin-like molecule comprises the amino acid sequence of SEQ ID NO: 48.
103. The method of embodiment 100, wherein the antibody, an antigen binding fragment or an immunoglobulin-like molecule comprises the amino acid sequence of SEQ ID NO: 49.
104. The method of embodiment 100, wherein the antibody, an antigen binding fragment or an immunoglobulin-like molecule comprises the amino acid sequence of SEQ ID NO: 46.
105. The method of embodiment 100, wherein the antibody, an antigen binding fragment or an immunoglobulin-like molecule comprises the amino acid sequence of SEQ ID NO: 51.
106. The method of embodiment 101, wherein the anti-CEA therapeutic comprises the amino acid sequence of SEQ ID NO: 48.
107. The method of embodiment 101, wherein anti-CEA therapeutic comprises the amino acid sequence of SEQ ID NO: 49.
108. The method of embodiment 101, wherein the anti-CEA therapeutic comprises the amino acid sequence of SEQ ID NO: 46.
109. The method of embodiment 101, wherein the anti-CEA therapeutic comprises the amino acid sequence of SEQ ID NO: 51.
110. The method of any of embodiments 6, 7, 8, 13-34, 45-48, 65-67, and 72-93, wherein said target is full-length CEA protein.
111. The method of any of embodiments 6, 7, 8, 13-34, and 45-48, 65-67, and 72-93, wherein said target is short form CEA protein.
112. A method of detecting CEA protein of any of embodiments 1-34, 36-41, and 51-60 further comprising detecting RNA encoding CEA protein of any of embodiments 61-111.

### EXEMPLIFICATION

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1

### Identification of Short Form CEA

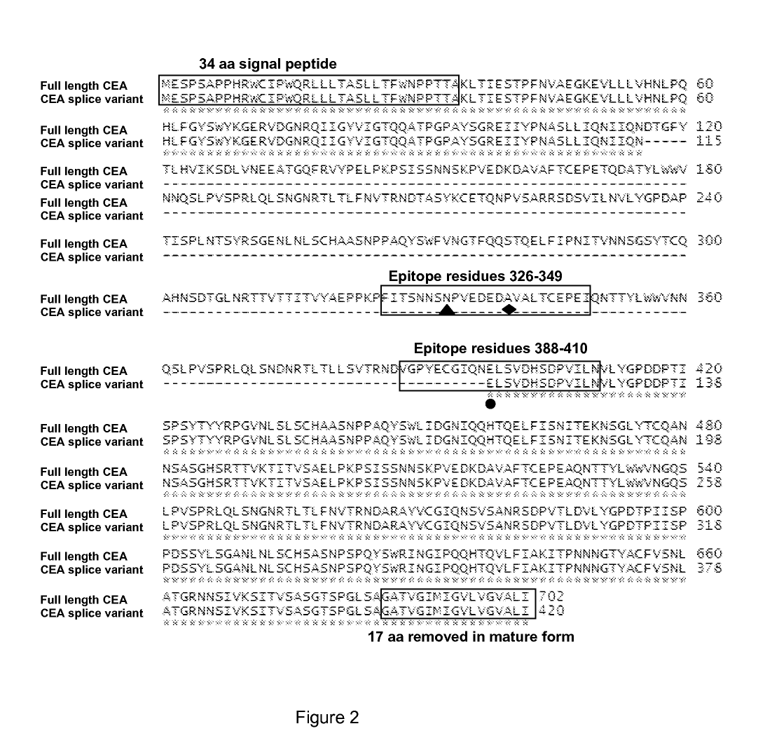

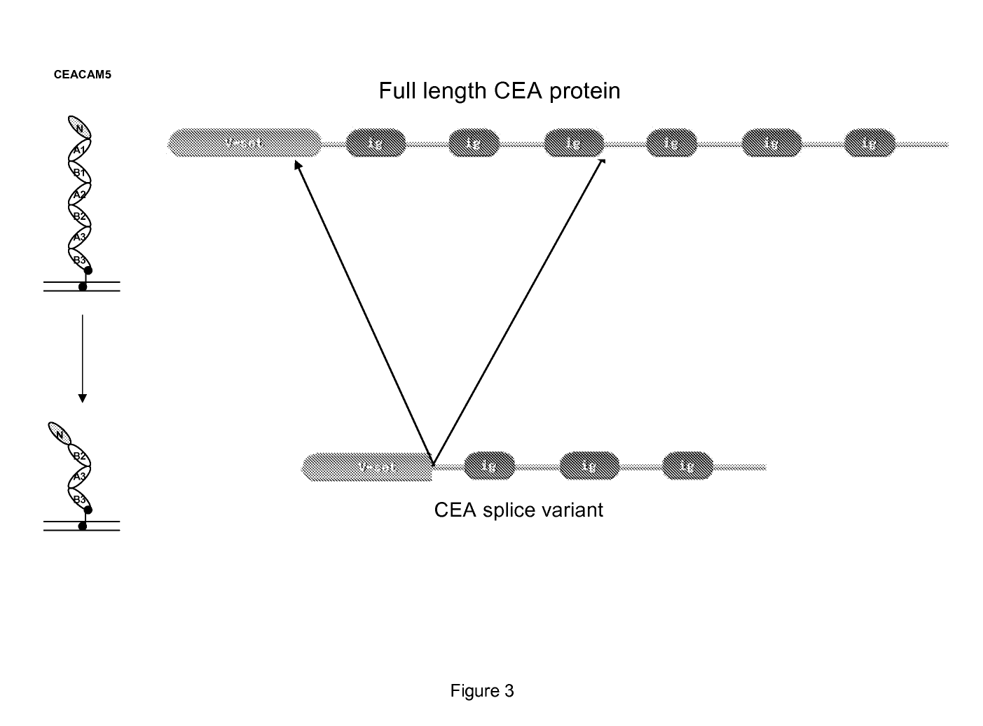

The full-length transcript of CEA (NCBI RefSeq NM_{—}002483; CEACAM5; CD66e) contains 10 exons; it translates into a protein precursor of 702 amino acids (aa) (NCBI RefSeq NP_{—}004354.2; FIGS. 1 and 2). The N-terminal 34 amino acid signal peptide and a 17 amino acid polypeptide in the C-terminus of CEA are removed during protein maturation (Bachman, 1987). A cDNA sequence of a shorter CEA splice variant (NCBI RefSeq CR749337; short CEA) from colonic cancer tissue was identified in the Genbank genetic sequence database. This transcript uses an alternative splicing donor site in exon 2 and skips exons 3 and 4; thus, the transcript results in a 420 aa protein with an in-frame truncation from aa 116 to 396 (as counted when including the N-terminal pro-sequence) of the full-length CEA (FIG. 2). The truncation deletes a small part of the V-set domain and three Ig C2-like domains (FIG. 3).

### Example 2

### Quantitative Analysis of Full-length CEA and Short Form CEA

TissueScan™ Disease Tissue quantitative polymerase chain reaction (qPCR) arrays were employed to determine the expression of full-length and short form CEA transcripts in normal and cancerous tissues of various stages and grades. Arrays included the TissueScan™ Colon Cancer Tissue qPCR Array I, TissueScan™ Pancreatic Cancer Tissue qPCR Array I, TissueScan™ Gastroesophageal Cancer Tissue qPCR Array I, TissueScan™ Lung Cancer Tissue qPCR Array I, and the TissueScan™ Breast Cancer Tissue qPCR Array I. For each tissue cDNA array, lyophilized cDNA was resuspended in 2.5 μL of ribonuclease—(RNase) free water. Plates were sealed, vortexed, and centrifuged to ensure resuspension of the full DNA sample. Primers and probes that specifically targeted the splice junctions of exons 2 and 5 (for the short CEA) and the splice junctions of exons 3 and 4 (for the full-length CEA) were used in analyses (Table 1).

TABLE 1
Primers and Probes Used in Taqman
Gene Expression Assays

| | | SEQ |
|---|---|---|
| | | ID |
| Primers and Probes | Sequence | NO: |
| CEA splice variant | CGCATACAGTGGTCGAGAGAT | 17 |
| forward primer | AATA | |
| CEA splice variant | CGCTGTGGTCAACACTTAATTT | 18 |
| reverse primer | GT | |
| CEA splice variant | 6FAM- | 19 |
| probe | ATGCATCCCTGCTGATCC- | |
| | MGBNFQ | |
| CEA full length | GAAACCCAGAACCCAGTGAGT | 20 |
| forward primer | | |
| CEA full length | GCCATAGAGGACATTCAGGAT | 21 |
| reverse primer | GAC | |
| CEA full length | 6FAM-CAGGCGCAGTGATTCA- | 22 |
| probe | MGBNFQ | |
| 18S Taqman ® Gene | Refer to Applied Biosystems | |
| Expression Assay | Assay ID No. Hs99999901_s1 | |

A pool of TaqMan assays was then created according to protocols supplied by the manufacturer for preamplification and subsequent amplification of the specific target transcripts. Specifically, 10 μL of 20× Custom TaqMan gene expression assay mix specific for the short CEA, full-length CEA, or ribosomal subunit 18S cDNA were combined with 990 μL of water to bring the final concentration of the assays to 0.2×. A preamplification reaction was prepared in the supplied 96 well plates by adding 2.5 μL of the pooled assay mix and 5 μL of TaqMan PreAmp Master Mix to the resuspended cDNA. The preamplification reactions were run on a Tetrad Thermalcycler under the conditions shown in Table 2. Following preamplification, samples were diluted to 100 μL by adding 90 μL of DNA resuspension buffer at pH 8.0. Quantitative PCRs were prepared with the preamplified cDNA samples and the TaqMan assay components for each of the target genes (Table 3).

TABLE 2
Preamplification Reaction Conditions for Quantitative
Polymerase Chain Reactions

| | Hold | Cycling (Cycle Number: 14) |
|---|---|---|
| Temperature/Time | 95° C./10 s | 95° C./15 s; 60° C./4 min |

TABLE 3
Components Used in Quantitative Polymerase Chain Reaction
Amplification Reactions

| Component | Volume (μL) |
|---|---|
| Taqman ® Gene Expression Assay (20X) | 1 |
| Pre-amplified cDNA product | 5 |
| Taqman ® Fast Universal PCR master mix | 10 |
| (2X) without AmpErase ® UNG | |
| RNAse/DNase free water | 4 |
| Total volume | 20 |

Following addition of all of the necessary reaction components, each plate was sealed with a MicroAmp Optical Adhesive Film, vortexed, briefly centrifuged, and then processed for qPCR in an ABI 7900HT Fast Real Time PCR Instrument in Fast Mode using the cycling conditions shown in Table 4.

TABLE 4
Amplification Reaction Conditions for Quantitative Polymerase
Chain Reactions

| | Hold | Cycling (Cycle Number: 40) |
|---|---|---|
| Temperature/Time | 95° C./20 s | 95° C./1 s; 60° C./20 s |

Upon completion of qPCR, SDS 2.2 Software was used to generate threshold cycle (Ct) values. In a typical reaction, PCR products are produced exponentially. Because it takes several cycles for the product to become detectable, the plot of fluorescence versus cycle number exhibits a sigmoidal appearance. At later cycles, the reaction substrates become depleted, PCR product no longer doubles, and the curve begins to flatten. The point on the curve at which the amount of fluorescence begins to increase exponentially, usually a few standard deviations above the baseline, is termed the Ct value. Relative quantification calculations were carried out after all the Ct values were exported. A Ct cutoff value of 30, below which the samples were considered to be positive and above which the samples were considered to be negative for gene expression, was arbitrarily selected.

As positive controls, cDNAs from Chinese hamster ovary (CHO) cells expressing either full-length (CHO/huCEA) or short form CEA (CHO/shortCEA clone 72-4) were used. Complementary DNAs were prepared by isolation of RNA with the RNeasy Mini Kit followed by cDNA synthesis utilizing the Superscript® one-cycle cDNA kit. The resulting cDNA was cleaned by RNA clean beads and was resuspended in 25 μL of RNase-free water. Negative control cDNA from parental CHO cells lacking the expression of dehydrofolate reductase (dhfr-CHO) that express neither full-length nor short form CEA was prepared in an identical fashion. Positive and negative control cDNAs (5 μL) were diluted 50-fold in RNase-free water and used as preamplified array cDNA in the qPCR amplification protocol described above. Positive control cDNA demonstrated Ct values <30 with their relevant gene-specific primers and probe sets, and Ct values >30 with non-relevant sets. As expected, Ct values >30 were observed for negative control cDNA with primers and probe sets for both full-length and short CEA. For the normalization of Ct values for calculation of relative expression levels of full-length and short CEA in a given sample, Ct values of ribosomal 18S RNA cDNA were used.

Using this methodology, TissueScan Disease Tissue qPCR arrays were used to determine the expression of full-length and short form CEA transcripts in normal and cancerous colonic, pancreatic, gastroesophageal, lung, and breast tissues (Table 5).

TABLE 5
Expression of Full-length and Short Form Carcinoembryonic
Antigen Transcripts in Normal and Cancerous Tissues

| | Full-length CEA | | Short Form CEA | |
|---|---|---|---|---|
| Tissue Type | Expression^{a} | | Expression^{b} | |
| Colon, normal | 5/5 | (100%) | 5/5 | (100%) |
| Colon, adenocarcinoma | 41/42 | (98%) | 41/42 | (98%) |
| Pancreas, normal | 3/4 | (75%) | 0/4 | (0%) |
| Pancreas, adenocarcinoma | 4/5 | (80%) | 1/5 | (20%) |
| Pancreas, islet cell cancer | 1/4 | (25%) | 0/4 | (0%) |
| Pancreas, neuroendocrine | 1/9 | (11%) | 0/9 | 0%) |
| cancer | | | | |
| Pancreas, acinar cellcancer | 0/1 | (0%) | 0/1 | (0%) |
| Gastroesophageal, normal | 6/6 | (100%) | 5/6 | (83%) |
| Gastroesophageal, cancer | 38/42 | (90%) | 21/42 | (50%) |
| Lung, normal | 8/8 | (100%) | 0/8 | (0%) |
| Lung, cancer | 39/40 | (98%) | 12/40 | (30%) |
| Breast, normal | 5/7 | (71%) | 0/7 | (0%) |
| Breast, adenocarcinoma | 38/41 | (93%) | 5/41 | (12%) |

^{a}Expression considered positive if Ct value ≦30.
^{b}Expression considered positive if Ct value ≦30.

Full-length CEA transcripts were commonly detected (Ct<30) in pancreatic adenocarcinomas (4 of 5), colonic (41 of 42), gastroesophageal (38 of 42), lung (39 of 40), and breast cancers (38 of 41) of various grades and stages. Full-length CEA transcripts were infrequently detected in neuroendocrine (1 of 9), islet cell (1 of 4), and acinar cell (0 of 1) pancreatic cancers. In normal colonic, lung, and gastroesophageal tissues, the full-length CEA transcripts were expressed at relatively high numbers (Ct<25). In contrast, in normal breast tissue, full-length CEA transcripts were expressed at lower numbers (25≦Ct≦30). Low expression (Ct<30) of full-length CEA transcripts was found in normal pancreatic tissues from TissueScan Arrays.

Expression (Ct<30) of short form CEA transcripts was generally found in colon (41 of 42) and in a half of gastroesophageal cancers (21 of 42). Expression was also seen in a proportion of lung (12 of 40) and breast cancers (5 of 41) of various grades and stages. Expression of short form CEA was infrequently found in pancreatic adenocarcinomas (1 of 5) and was not found in neuroendocrine (0 of 9), islet cell (0 of 4) or acinar cell pancreatic cancers (0 of 1). Expression of short form CEA transcripts was always concordant with expression of full-length CEA transcripts. These results show that expression of short form CEA splice transcripts varies in different cancers, but is always coexpressed with the full-length CEA transcripts.

### Example 3

### Cloning and Expression of Short Form CEA in Chinese Hamster Ovary Cells

The plasmid for cDNA clone DKFZp781M2392Q containing the sequence for short form CEA was purchased from ImaGenes GmbH in association with B Bridge International. Plasmid DNA was prepared from bacterial culture by plasmid DNA purification using a QIAprep spin miniprep kit. Purified DNA was subsequently used as a template in a PCR reaction (Tables 6 and 7) to amplify the short form CEA using the Herculase II Fusion DNA polymerase kit and gene-specific primers 376 and 377 (Table 8).

TABLE 6
Polymerase Chain Reaction Cycling Conditions for
Amplification of Short Carcinoembryonic Antigen

| | | Cycling Cycle | | |
|---|---|---|---|---|
| | Hold | Number: 30 | Hold | Hold |
| Temperature/Time | 95° C./4 min | 95° C./30 s; | 72° C./7 min | 4° C. |
| | | 60° C./30 s; | | |
| | | 72° C./2 min | | |

TABLE 7
Gene Specific Primers Used in Subcloning
Polymerase Chain Reactions of Short
Carcinoembryonic Antigen

| Primer | Sequence (5′ to 3′) |
|---|---|
| 376 | TACCGCTAGCGCCACCATGGAGTCTCCCTCGGCCCCT |
| | CCC (SEQ ID NO: 23) |
| 377 | GCTCGAATTCTCATATCAGAGCAACCAACCAGC (SEQ |
| | ID NO: 24) |

TABLE 8
Polymerase Chain Reaction Components for Amplification of
Short Carcinoembryonic Antigen

| Component | Volume (μL) |
|---|---|
| 5× Reaction Buffer | 10 |
| (100 mM Tris HCl, pH 8.8; 10 mM MgSO4; | |
| 50 mM KCl; 50 mM (NH4)2SO4; 0.5% | |
| Triton X-100; | |
| 0.5 mg/mL bovine serum albumin) | |
| 10 nM dNTP | 1 |
| 0.1 mg/mL CEA splice variant DNA | 1 |
| 10 μM primer 376 | 1 |
| 10 μM primer 377 | 1 |
| Herculase II polymerase, 2.5 U/μL | 1 |
| H₂O | 35 |
| Taq polymerase, 5 U/μL (added after cycle | 1 |
| 27) | |
| Total volume | 51 |

The reaction product was detected as 1.2 kilo base DNA band after 1% TAE (Tris acetate ethylenediaminetetraacetic acid) gel electrophoresis and was purified from the gel using a QIAquick PCR purification kit. The product was cloned by TOPO® TA Cloning® into the pCR2.1 vector and transformed into TOP10 bacteria that were subsequently screened by blue/white selection and diagnostic PCR using CEA gene specific primers 376 and 377. The DNA from positive colonies was sequenced by a Prism Big Dye Terminator to confirm the correct short CEA sequence. The verified short form CEA was subcloned into pCDH1-HCS1-EF1-Puro vector by ligation of NheI/EcoRI digested CEA splice variant into NheI/EcoRI-digested virus using a Quick Ligation Kit™. Such subcloning positioned the short CEA downstream of the cytomegaloviral promoter that directs constitutive expression of the gene. The lentiviral vector carrying the short form CEA was transformed into TOP10 bacteria, and bacterial colonies were screened by PCR using primers specific for lentiviral and short form CEA sequence. A positive colony containing the short form CEA in the correct orientation was used to prepare lentiviral DNA with the Rapid Plasmid Maxiprep System. Purified lentiviral DNA was transfected together with a packaging plasmid into 293T cells by Lipofectamine™2000 transfection reagent to produce infectious lentivirus. Cell culture media containing the lentivirus was used for infection of dhfr-CHO cells. The cells were subsequently treated with 5 μg/mL of puromycin in complete growth media for either 72 hours or 2 weeks for the selection of lentiviral-infected CHO cells expressing the short form CEA. Pooled clones of cells selected by puromycin were cloned as single cells by the limiting dilution technique. Single-cell derived clones expressing various levels of the short form CEA protein were identified by an antibody binding assay as defined in Example 4.

### Example 4

### Antibody Binding

For binding of IgG B9 (IgG B9 is a chimeric antibody with (a) humanized CDRs of the mouse A5B7 and (b) a mouse IgG1 Fc domain) and anti-CEA antibodies (anti-CEACAM 5 monoclonal antibody, FITC conjugated anti-human CD66 a, c, d, e, [CEACAM1, 3, 5, 6], and anti-CEACAM1, 3, 4, 5, 6), the antibodies were incubated with cells at a concentration of 10 μg/mL diluted in fluorescence activated cell sorting (FACS) buffer (phosphate buffered saline [PBS]+2% fetal bovine serum [FBS]) for 20 minutes at 4° C. For the detection of bound IgG B9 and unlabeled anti-CEA monoclonal antibodies, the cells were washed twice with FACS buffer and incubated with 10 μg/mL of goat anti-mouse AlexaFluor®488 secondary antibody in FACS buffer for 15 minutes at 4° C. in the dark. Before the determination of fluorescence intensities, the cells were washed one time with FACS buffer. Binding of antibodies to T cells was performed in FACS buffer using antibodies against CD4, CD8, and CD25 at concentrations recommended by the manufacturer. For the binding assays, the cells were resuspended in FACS buffer and incubated with antibodies and 10 μg/mL of propidium iodide (PI) for 20 minutes at 4° C. in the dark. The fluorescence intensities of bound secondary or directly conjugated antibodies were determined by flow cytometry using an LSRII flow cytometer; and the data were analyzed using FlowJo Software. Viable cells were examined for antibody binding following fluorescence compensation by single-stained control cells to establish compensation matrices. Fluorescence-minus-one controls were included for the gating of negative and positive staining events.

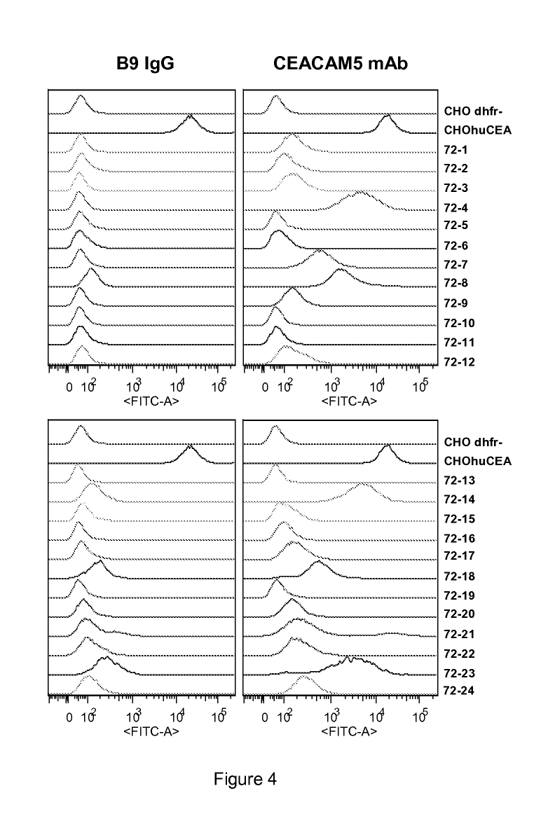

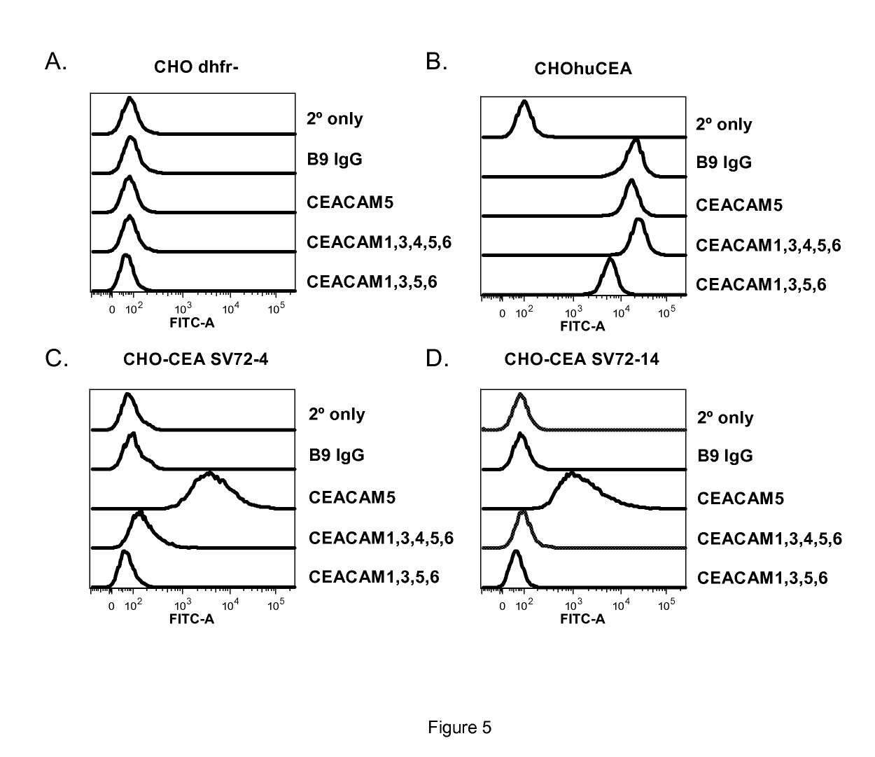

Generation of CHO cells expressing the short form CEA is described in Example 3. Single cell clones were screened for the cell surface expression of short form CEA protein by binding of IgG B9 or CEACAM5-specific MAb (FIG. 4). Anti-CEACAM5 MAb bound to numerous clones at various levels. In contrast, IgG B9 did not substantially bind to any clones. Two clonal lines, 72-4 and 72-14, that lost the epitope recognized by IgG B9 and also lost binding to two other anti-CEA monoclonal antibodies (FIG. 5), were chosen for further study. These clones also showed the highest levels of anti-CEACAM5 specific monoclonal antibody binding (FIG. 4).

### Example 5

### Cellular Cytotoxicity

Effector T cell-mediated killing of CHO/huCEA cells was determined using a flow cytometry-based assay that measures the viability of dye-labeled target cells. Effector human CD3+ T cells were isolated from heparinized whole blood of healthy donors by the RosetteSep® human T cell enrichment cocktail and RosetteSep T cell density medium. Briefly, 20 mL of human whole blood was added to 1 mL of human T cell enrichment cocktail, mixed, and incubated at 25° C. for 20 minutes. The blood was then diluted 1:1 with FACS buffer and centrifuged on RosetteSep T cell density medium. Enriched human T cells were removed at the serum/density medium interface and washed two times with FACS buffer. Finally, CD3+ enriched T cells were resuspended in Roswell Park Memorial Institute (RPMI)-1640 cell culture medium containing 10% FBS and 4.5 g/L glucose at a density of 2.8×10⁶ cells/mL.

CHO/huCEA cells (expressing full-length CEA protein) or CHO/shortCEA clones 72-4 and 72-14 (each of which express short form CEA protein) were used as target cells. Cells were harvested, washed, and finally resuspended in FACS buffer at a density of 1×10⁶ cells/mL. Cells were quickly added to 5 μL of 3,3′-dioctadecyloxacarbocyanine (DiO) cell labelling solution per mL and incubated for 1 minute at 37° C. Cells were washed and resuspended in RPMI-1640 cell culture medium at a density of 2.8×10⁵ cells/mL.

Serial dilutions of BiTE antibodies (20 μL) were added to 90 μL each of target and effector cells in 96 well non-tissue culture treated plates at an effector-to-target ratio of 10 to 1. MEC14 BiTE was used as a control BiTE for MEDI-565. MEC14 BiTE is composed of a murine anti-Mecoprop (an herbicide) single-chain antibody linked to the same anti-CD3 single-chain antibody used to construct MEDI-565. The BiTE-mediated cytotoxic reactions were carried out at 37° C. in an atmosphere with 5% CO₂ for 72 hours.

Cytotoxicity, defined as a loss of cell membrane integrity, was monitored by cellular intake of PI. Viable cells exclude PI, whereas PI stains nucleic acids in nonviable cells. The percentage of nonviable propidium iodide-positive, DiO-labeled target cells was determined by flow cytometry using an LSRII flow cytometer, and the data were analyzed using FlowJo software.

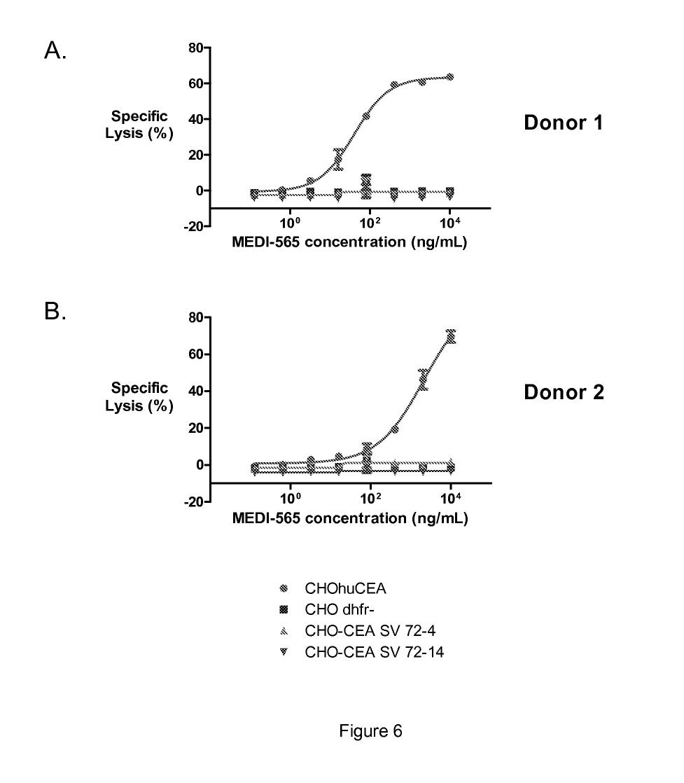

MEDI-565 mediated dose-dependent killing of CHO/huCEA cells (cells expressing full-length CEA protein). In contrast, MEDI-565 did not mediate killing of CHO/shortCEA cell clones 72-4 and 72-14 (cells expressing short form CEA protein) (FIG. 6). As anticipated, MEDI-565 did not mediate killing of parental dhfr-CHO cells lacking expression of CEA.

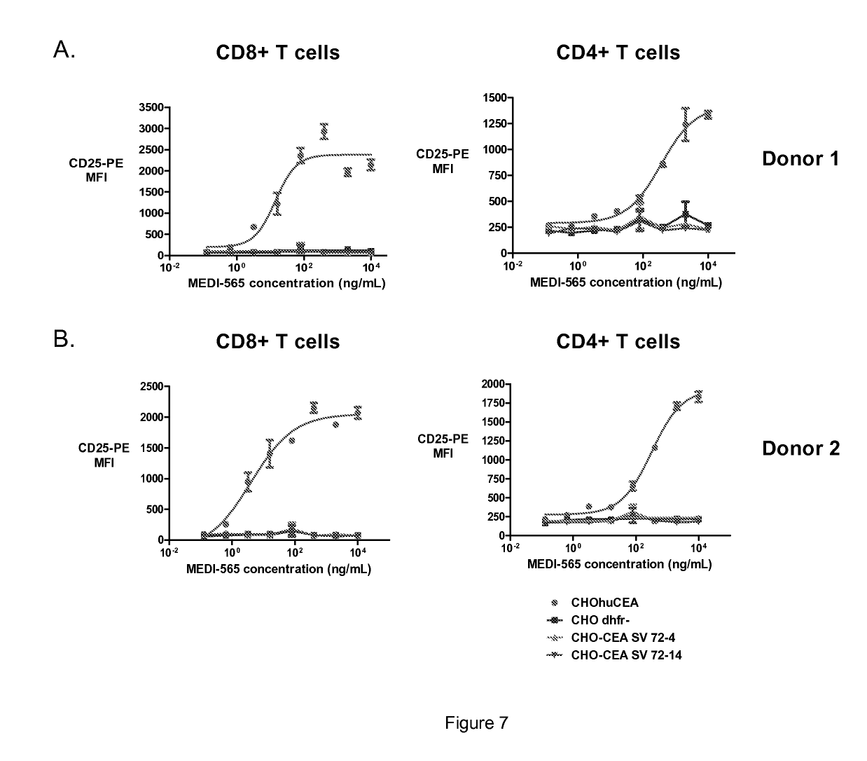

MEDI-565 induced expression of the surface antigen CD25 on human CD4+ and CD8+ T cells in a dose-dependent manner following coculture with CHO/huCEA cells (FIG. 7). This effect on CD4+ and CD8+ T cells was absent in the presence of CHO/shortCEA cell clones 72-4 and 72-14. In addition, MEDI-565 did not activate human CD4+ and/or CD8+ T cells cultured with parental dhfr-CHO cells lacking expression of CEA. These results are consistent with the inability of MEDI-565 to bind to or to meditate T cell killing of the cells expressing only short form CEA protein.

The following conclusions can be drawn from Examples 1-5. Full-length CEA transcripts were widely expressed in colonic, pancreatic, gastroesophageal, lung, and breast cancers. Short form CEA transcripts were widely expressed in colonic cancers, sparsely in gastroesophageal, lung, and breast cancers, and seldom in pancreatic cancers. However, although expression levels and frequency differed, expression of short form CEA transcripts in all tissue samples was concordant with expression of the full-length CEA transcripts.

Transduction of parental dhfr-CHO cell line lacking expression of CEA with short form CEA yielded single cell clones expressing short form CEA protein and that bound a MAb specific for CEACAM5. However, the short form CEA protein was not bound by IgG B9, a murine monoclonal antibody that shares the same CEA-binding moiety with MEDI-565, or by two separate monoclonal pan-CEA antibodies (CD66a,c,d,e/CEACAM1,3,5,6 and CEACAM1,3,4,5,6). This result indicates that the short form CEA protein does not retain the specific epitope that is present in the full-length CEA protein and recognized by IgG B9. As anticipated, MEDI-565 did not mediate the T cell-directed killing of CHO/shortCEA cells (CHO cells expressing only short form CEA protein), and T cells were not activated in these cytotoxicity assays. This stands in contrast to MEDI-565 mediated killing of CHO/huCEA cells with concomitant T cell activation.

Collectively, the results presented herein suggest that the expression of short CEA will not interfere with the binding of MEDI-565 to full-length CEA on the cell surface. Furthermore, the presence of short form CEA will not impede MEDI-565-mediated T cell killing of cancer cells expressing CEA.

### Example 6

### Epitope Mapping Through Deletion Mutants

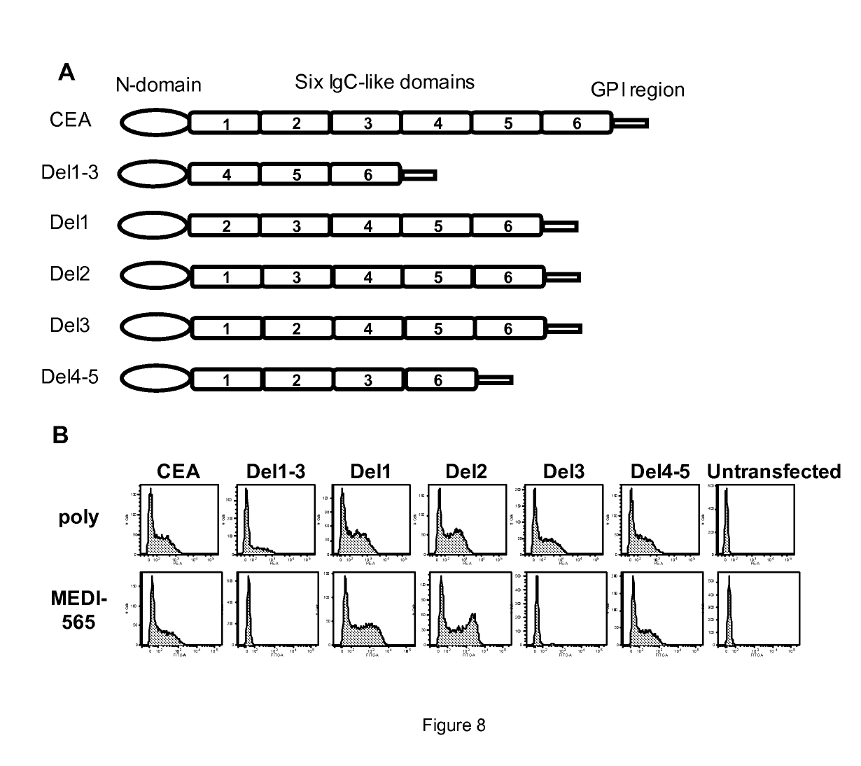

Full-length human CEA precursor protein encoded with 702 amino acids (aa) is comprised of a 34-amino acid N-terminal signal peptide (removed in mature protein), one IgV-like N-domain, six IgC-like domains, and a 17 amino acid C-terminal peptide (removed in mature protein). The amino acid sequence of the mature protein of full-length human CEA is SEQ ID NO:2. To identify the domain of CEA recognized by MEDI-565, CEA deletion mutants were engineered by truncating IgC-like domains. Five deletion mutants were constructed as shown in FIG. 8: deletion of IgC-like domain 1-3 (Dell-3, SEQ ID NO:3), deletion of IgC-like domain 1 (Dell, SEQ ID NO:4), deletion of IgC-like domain 2 (Del2, SEQ ID NO:5), deletion of IgC-like domain 3 (Del3, SEQ ID NO:6), and deletion of IgC-like domain 4-5 (Del4-5, SEQ ID NO:7).

The deletion mutants were amplified and assembled through overlapping extension PCR using an in-house full-length CEA clone as template. Both full-length and deletion mutants were cloned into a mammalian expression vector with a human cytomegalovirus major immediate early (hCMVie) enhancer, promoter and 5′-untranslated region.

The deletion mutants were expressed as membrane bound GPI-anchored proteins by transient transfection into HEK293 F cells using 293fectin. Transfectants were incubated with MEDI-565 followed by an anti-penta-His antibody labeled with Alexa Fluor 488, then screened for binding using the LSRII flow cytometer. Protein expression of all constructs was monitored with an anti-CEA polyclonal antibody.

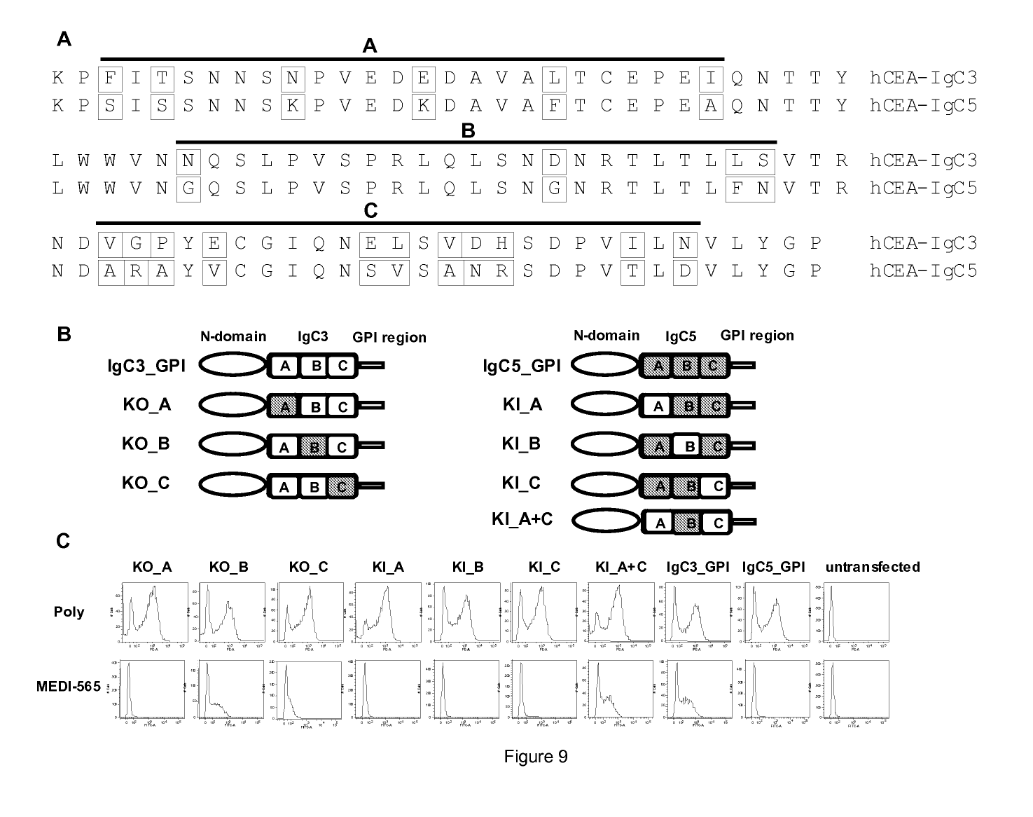

The epitope of MEDI-565 was mapped to the IgC3 domain of CEA using the domain deletion mutants. Deletion of the IgC3 or IgC1-3 domains disrupted the binding of MEDI-565 to CEA (FIG. 8). The other deletion mutants retained MEDI-565 binding (e.g., MEDI-565 binding was not disrupted when the IgC1, 2, or 4-5 domains were deleted). Furthermore, a truncated CEA protein (IgC3_GPI) encoding the IgC3 domain, a flanking N-domain and the GPI region retained MEDI-565 binding equal to the full-length CEA protein (FIG. 9).

### Example 7

### Epitope Mapping Using Swap Mutants

Swap mutants were constructed exchanging short segments of the third IgC-like domain (IgC3) of CEA with the fifth IgC-like domain (IgC5), and such swap mutants were evaluated for MEDI-565 binding. Because IgC5 is not involved in MEDI-565 binding but shares high homology with IgC3, it was selected for use in knocking-out each segment of IgC3 (FIG. 9). Two truncated CEA proteins were constructed as templates for swap mutants, IgC3_GPI (SEQ ID NO:8) and IgC5_GPI (SEQ ID NO:9; FIG. 9). Each construct is composed of the N-domain, the IgC3 or the IgC5 domain, and the GPI region, but these constructs do not encode the other five IgC-like domains (FIG. 9).

The IgC3 and IgC5 domains were divided into three segments of approximately 30-amino acids, and each segment was denoted as segments A, B, and C (FIG. 9). For “knock-out” mutants (KO), IgC3_GPI was used as a template in which segments of IgC3 were substituted with the corresponding segments of IgC5 to engineer KO_A, KO_B, and KO_C (FIG. 9). For “knock-in” mutants (KI), IgC5_GPI was used as a structural template in which IgC3 segments were grafted in place of IgC5 counterparts, constructing KI_A, KI_B, KI_C, and KI_A+C (FIG. 9).

Swap mutants were assembled using overlapping extension PCR and cloned into a mammalian expression vector encoding a human cytomegalovirus major immediate early (hCMVie) enhancer, promoter and 5′-untranslated region for transient mammalian expression.

The swap mutants were expressed as membrane bound GPI-anchored proteins by transient transfection into HEK293F cells. Transfectants were incubated with MEDI-565 followed by an anti-penta-His monoclonal antibody labeled with Alexa Fluor 488, then screened for binding using the LSRII flow cytometer. Protein expression of all constructs was monitored with an anti-CEA polyclonal antibody.

Two segments of the IgC3 domain were found to be important for MEDI-565 binding. Substitution of segments A (292-315) or C (354-376) of IgC3_GPI with the corresponding IgC5 segments abolished the binding of MEDI-565 (numbering of segments is with respect to mature, full-length CEA protein). Consistently, grafting both segments A and C of IgC3 into IgC5_GPI resulted in MEDI-565 binding. Segment B had no impact on binding. Therefore, the studies using swap mutants revealed that MEDI-565 binds to a nonlinear epitope in the IgC3 domain of CEA comprised of segments A (residues 292-315) and residues C (354-376). As evident from the above analysis of short form CEA, this epitope is not present in short form CEA protein.

### Example 8

### Epitope Mapping Through Site-Directed Mutagenesis and Computational Homology Modeling

Site-directed mutagenesis and computational homology modeling were implemented to identify residues of CEA critical for MEDI-565 binding. Because the following residues are localized in the IgC3 epitope-containing regions of segment A and segment C, and because these residues differ between IgC3 and IgC5, they were mutated as clusters to encode the corresponding IgC5 amino acids: F²⁹²T²⁹⁴N²⁹⁹, N²⁹⁹E³⁰⁴, E³⁰⁴L³⁰⁹I³¹⁵, V³⁵⁴G³⁵⁵P³⁵⁶E³⁵⁸, E³⁶⁴L³⁶⁵V³⁶⁷D³⁶⁸H³⁶⁹, and I³⁷⁴N³⁷⁶ (numbering of mature protein).

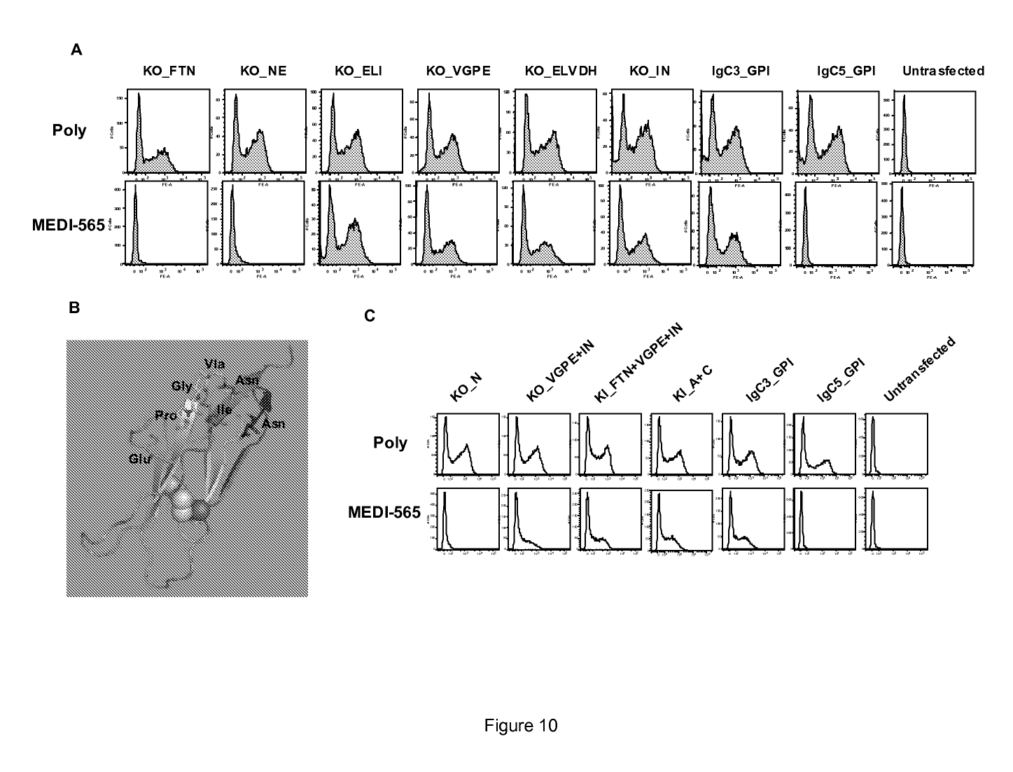

A modeled structure of IgC3 domain of CEA was constructed using the crystal structure of murine CEACAM1A (33.7% sequence homology) as a template using the SWISS-MODEL workspace (FIG. 10). Based on the results from the site-directed mutagenesis and the modeled structure, three additional mutants were constructed, replacing N²⁹⁹ of IgC3 with the corresponding residue of IgC5 (KO_N), substituting V³⁵⁴G³⁵⁵P³⁵⁶E³⁵⁸ and I³⁷⁴N³⁷⁶ of IgC3 with the counterparts of IgC5 (KO_VGPE+IN), and grafting F²⁹²T²⁹⁴N²⁹⁹, V³⁵⁴G³⁵⁵P³⁵⁶E³⁵⁸, and I³⁷⁴N³⁷⁶ of IgC3 into IgC5 (KI_FTN+VGPE+IN).

All mutants were assembled using overlapping extension PCR using the truncated CEA proteins IgC3_GPI or IgC5_GPI as templates. The various mutant constructs were cloned into a mammalian expression vector encoding a human cytomegalovirus major immediate early (hCMVie) enhancer, promoter and 5′-untranslated region for transient mammalian expression.

The mutant constructs were expressed as GPI-anchored proteins by transient transfection into HEK293 F cells. Transfectants were incubated with MEDI-565 followed by an Alexa Fluor 488 labeled anti-penta-His Mab, then screened for binding using the LSRII flow cytometer. Protein expression of all constructs was monitored with an anti-CEA polyclonal antibody.

When residues F²⁹²T²⁹⁴N²⁹⁹ in IgC3 segment A were replaced with the corresponding residues of IgC5, the binding of MEDI-565 was disrupted (FIG. 10) (numbering of mature protein). Particularly, residue N²⁹⁹ was identified as a critical hot spot residue since knocking-out this single amino acid in IgC3 abolished MEDI-565 binding.

The residues V³⁵⁴, G³⁵⁵, P³⁵⁶, E³⁵⁸, I³⁷⁴, and N³⁷⁶ in segment C of IgC3 are also involved in MEDI-565 binding. The modeled structure of IgC3 reveals two clusters of residues (V³⁵⁴G³⁵⁵P³⁵⁶E³⁵⁸ and I³⁷⁴N³⁷⁶) in segment C spatially close to the critical residue N²⁹⁹ of segment A (FIG. 10) (numbering of mature protein). The model suggested that these residues in segment C could contribute to MEDI-565 binding. In fact, knocking-out these residues together significantly affected (but did not completely abolish) MEDI-565 binding. Finally, grafting of amino acids F²⁹²T²⁹⁴N²⁹⁹ of segment A in combination with amino acid V³⁵⁴G³⁵⁵P³⁵⁶E³⁵⁸ and I³⁷⁴N³⁷⁶ of segment C into IgC5 resulted in MEDI-565 binding; the level of this binding was comparable to that of the knock-in mutant of both segments A and C together (a total of 60 amino acid residues knocked-in) (FIG. 10).

The following conclusions can be drawn from Examples 6-8. With a combination of deletion and swap mutant analysis, site directed mutagenesis, and computational homology modeling, we mapped a nonlinear, conformational epitope of MEDI-565 in the third immunoglobulin C-like domain of CEA, comprised of two regions of residues 292-315 and 354-376, with the hot spot critical residue of N²⁹⁹ (numbering of mature protein). Residues F²⁹², T²⁹⁴, V³⁵⁴, G³⁵⁵, P³⁵⁶, E³⁵⁸, I³⁷⁴, and N³⁷⁶ are also important for the binding of MEDI-565 (numbering of mature protein). This epitope is present in full-length CEA protein, but not in short form CEA protein (the CEA splice variant described herein). Note that the above numbering of residues in CEA is shown with respect to the mature protein (e.g., the counting of residues does not include N-terminal pro-sequence). However, these residues can also be identified with respect to the pro-protein (e.g., the counting of residues does include the N-terminal pro-sequence) by adding 34 to the residue numbers shown in, for example, Example 8.

### Example 9

### Review of CEA Polymorphisms

To identify other possible CEA variants, amino acid polymorphisms were surveyed using the NCBI single-nucleotide polymorphisms (SNP) database (http://www.ncbi.nlm.nih.gov/projects/SNP/). Ten individual, non-synonymous coding polymorphisms were identified in full-length CEA protein, as shown in the table below.

TABLE 9

| | | | Amino Acid Position in |
|---|---|---|---|
| | | | Mature, Full-Length |
| | | | CEA (corresponding |
| | | | position if the N- |
| | Nucleotide | Amino Acid | terminal pro-sequence is |
| SNP ID | Change | Change | included) |
| rs12971352 | A/G | I/V | 46 (80) |
| rs28683503 | T/C | V/A | 49 (83) |
| rs34155934 | A/G | I/V | 78 (112) |
| rs35091611 | T/C | I/T | 79 (113) |
| rs3815780 | A/C | Q/P | 103 (137) |
| rs17853315 | A/T | T/S | 176 (210) |
| rs10407503 | C/A | A/D | 306 (340) |
| rs7249230 | A/G | K/E | 364 (398) |
| rs10423171 | C/A | R/S | 630 (664) |
| rs11545767 | G/A | G/R | 644 (678) |

Two non-synonymous coding SNPs (rs10407503, rs7249230) were identified in the MEDI-565 binding epitope regions. The single-nucleotide change from C to A in rs10407503 resulted in an amino acid change from alanine (Ala or A) to aspartic acid (Asp or D) at amino acid position 306 of mature, full-length CEA. The single-nucleotide change from A to G in rs7249230 resulted in an amino acid change from lysine (Lys or K) to glutamic acid (Glu or E) at amino acid position 364 of mature, full-length CEA.

According to the SNP database, the average heterozygosity rates in the population are 0.141 and 0.168 for rs10407503 and rs7249230, respectively. However, the minor allele homozygotes rate for rs10407503 is close to 0 in both European and Asian and is 0.08 in Sub-Saharan African populations. The minor allele homozygotes rate for rs7249230 was from 0 to 0.03 in different populations. Since the homozygotes rates of both SNPs are very low and they do not affect the critical residues involved in MEDI-565 binding, we anticipate little to no impact of these SNPs on the binding of MEDI-565 to CEA.

### Example 10

### Determining Susceptibility to Anti-CEA Cancer Therapy

A patient with CEA expressing cancer is eligible for testing of susceptibility to anti-CEA cancer treatment. A cancer therapeutic is selected for testing. The cancer therapeutic immunospecifically binds to full-length form of CEA protein (cell surface and secreted full-length CEA protein), the target CEA, but does not immunospecifically bind to the short form of CEA protein (in this example, a non-target CEA). A sample, such as a blood sample, is taken from the patient and analyzed with an ELISA assay specific to the target CEA protein to determine the concentration of the target CEA protein without detecting the concentration of other non-target forms of CEA protein. The concentration of the target CEA protein is compared to a standard range reflecting target CEA protein concentration in samples from healthy subjects. If the target CEA protein concentration is above the standard range, the patient is susceptible to a cancer therapeutic that immunospecifically binds to the target CEA protein. Optionally, other parameters are also evaluated to determine if the cancer treatment protocol is favorable for the patient.

Alternatively, or in addition, a sample, such as a blood sample, is taken from the patient and analyzed with a method to detect RNA encoding full-length and/or short form CEA protein. The concentration(s) of the target CEA RNA is compared to a standard range reflecting target CEA RNA concentration in samples from healthy subjects. If the target CEA RNA concentration is above the standard range, the patient is susceptible to a cancer therapeutic that immunospecifically binds to the target CEA protein. Optionally, other parameters are also evaluated to determine if the cancer treatment protocol is favorable for the patient.

It is expected that subjects with cancer will be susceptible to certain therapies targeted to specific forms of CEA described in this application.

Diagnostic monitoring of a patient determined to be susceptible to a cancer therapeutic that immunospecifically binds to a target CEA protein is with diagnostic methods based on detection of target CEA concentration and/or CEA RNA concentration at various time periods after treatment (either after specific treatments or after conclusion of the entire regimen). For example, diagnostic monitoring is with an antibody, antigen binding fragment or immunoglobulin-like molecule that immunospecifically binds the target CEA. In certain embodiments, the diagnostic antibody, antigen binding fragment or immunoglobulin-like molecule immunospecifically binds to the same or substantially the same epitope on target CEA as the cancer therapeutic.

Depending on the particular type of CEA-expressing cancer, target CEA concentration can be monitored by taking samples of other fluids. For example, particularly for colon cancer and rectal cancer, blood or fecal samples can be taken and used to monitor CEA concentration prior to, during, and/or following treatment. By way of further example, for lung cancer, sputum samples can be similarly used.

### Example 11

### Specificity for Full-Length CEA

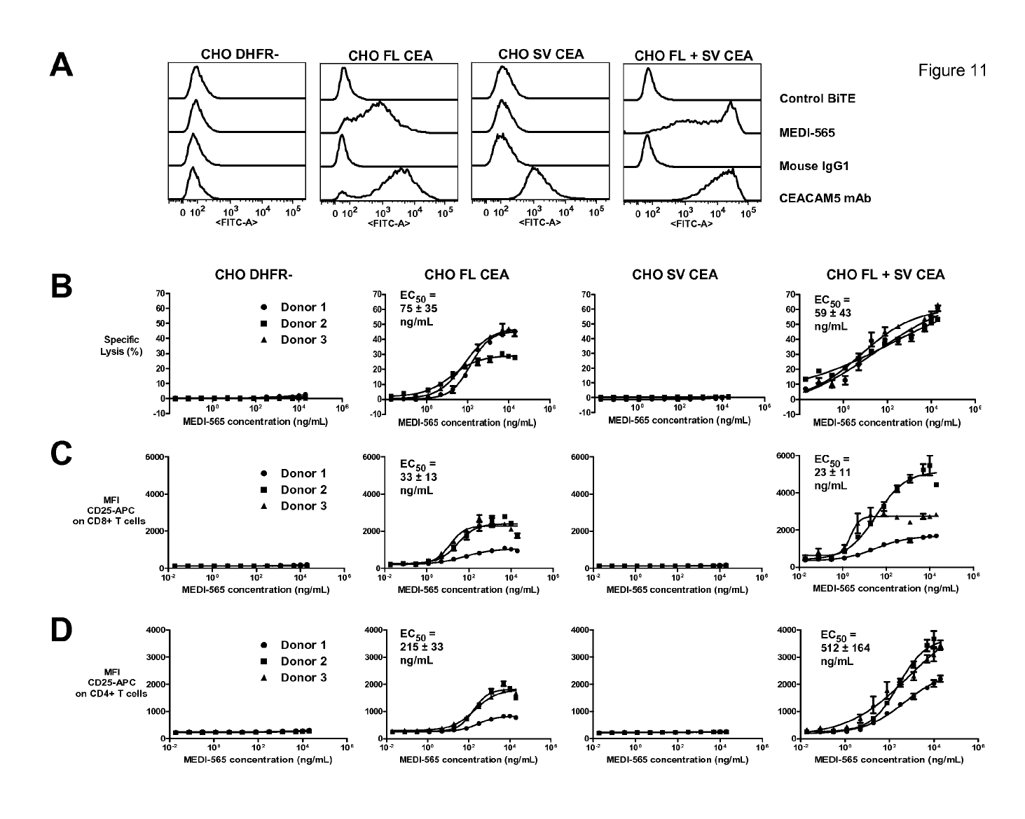

The amino acids in full-length CEA important for MEDI-565 binding were found to be largely absent in the CEA splice variant, except amino acids I⁴⁰⁸ and N⁴¹⁰ in segment C (FIG. 2, shown for proteins that include the signal sequence; note these residues correspond to I³⁷⁴ and N³⁷⁶ when counting only with respect to the mature protein.). In fact, the knock-out (KO_A) and knock-in (KI_C) mutants constructed for epitope mapping, which have segment C (containing amino acids I⁴⁰⁸ and N⁴¹⁰), but which lack of A (containing the residue N³³³) (numbering of full length CEA protein), were not recognized by MEDI-565 (FIG. 9). This observation suggested that binding of MEDI-565 to the CEA splice variant was unlikely to occur. To test this hypothesis, CHO cells were infected with lentivirus constructs directing the expression of full-length CEA (CHO FL CEA) or the CEA splice variant (CHO SV CEA), or both sequentially (CHO FL+SV CEA); CEA protein expression was verified by western blotting (data not shown). As anticipated, MEDI-565 bound to cells expressing full-length CEA but not to cells expressing the CEA splice variant (FIG. 11A). Zhou et al. showed that the IgV-like domain and IgC5 mediate homophilic CEA interactions on apposed cell surfaces; both domains are present within the CEA splice variant. Thus, we wanted to test the hypothesis that co-expression of the CEA splice variant (short-form) and full-length CEA proteins may, through homophilic interactions, result in the MEDI-565 binding epitope being masked in the full-length form and subsequently prevent MEDI-565 binding to full-length CEA. Co-expression of the CEA splice variant (short-form) with full-length CEA on the same cells did not significantly affect the binding affinity of MEDI-565 to full-length CEA (CHO FL CEA, K_{D}=5.0±0.15 nM; CHO FL+SV CEA, K_{D}=5.6±3.0 nM; p=0.86). Homophilic interactions do not appear to prevent MEDI-565 binding.

Consistent with the MEDI-565 binding data, CHO SV CEA did not mediate the activation of T cells from healthy donors, nor were un-stimulated human T cells induced to lyse CHO SV CEA cells when cultured with MEDI-565 (FIG. 11). In contrast, MEDI-565 activated T cells and induced killing of CHO FL CEA or CHO FL+SV CEA cells with similar levels of activity (EC₅₀ values); CHO FL CEA, EC₅₀=75±35 ng/mL; CHO FL+SV CEA, EC₅₀=59±43 ng/mL; p=0.79. Since MEDI-565 does not bind the CEA splice variant, the co-expression of CEA splice variant and full-length CEA is not anticipated to interfere with MEDI-565 binding to full-length CEA, nor inhibit MEDI-565-mediated T-cell killing of cancer cells expressing full-length CEA. However, discrimination of the full-length CEA from the CEA splice variant may be important while monitoring the status of CEA positive tumor cells via changes in serum CEA levels in patients that receive MEDI-565 therapy.

### Example 12

### Treatment of CEA Expressing Cancer

A patient with CEA expressing cancer is eligible for treatment. Optionally, the patient has been shown to be susceptible to a cancer therapeutic that immunospecifically binds to a target CEA protein as shown in Example 10. A sample, such as a blood sample, is taken from the patient and analyzed with an ELISA assay specific to the target CEA protein, full-length CEA, to determine the concentration of the target CEA protein without detecting the concentration of other non-target forms of CEA protein. Alternatively, a sample, such as a blood sample, is taken from a patent and analyzed for the concentration of RNA encoding full-length and/or short form CEA.

The patient is treated with the cancer therapeutic, MEDI-565, that immunospecifically binds to the target CEA protein. Optionally, a combination of therapeutics, including therapeutics like surgery, chemotherapy, and radiation that are not specific to CEA expression, is used in the treatment. A post-treatment blood sample is taken from the patient and tested for the concentration of the target CEA protein without detecting the concentration of other non-target forms of CEA protein. The concentration of the target CEA protein is compared to the pre-treatment target CEA protein concentration from the same patient.

It is expected that the change in target CEA protein concentration will indicate the effectiveness of the therapy.

Diagnostic monitoring of a patient determined to be susceptible to a cancer therapeutic that immunospecifically binds to a target CEA protein is with diagnostic methods based on detection of target CEA concentration at various time periods after treatment (either after specific treatments or after conclusion of the entire regimen). For example, diagnostic monitoring is with an antibody, antigen binding fragment or immunoglobulin-like molecule that immunospecifically binds the target CEA. In certain embodiments, the diagnostic antibody, antigen binding fragment or immunoglobulin-like molecule immunospecifically binds to the same or substantially the same epitope on target CEA as the cancer therapeutic.

Depending on the particular type of CEA-expressing cancer, target CEA concentration can be monitored by taking samples of other fluids. For example, particularly for colon cancer and rectal cancer, blood fecal samples can be taken and used to monitor CEA concentration prior to, during, and/or following treatment. By way of further example, for lung cancer, sputum samples can be similarly used.

### REFERENCES

Behr T M, Liersch T, Greiner-Bechert L, et al. Radioimmunotherapy of small-volume disease of metastatic colorectal cancer. Cancer. 2002; 94:1373-81.
Duffy, M J. Carcinoembryonic antigen as a marker for colorectal cancer: Is it clinically useful?Clinical Chemistry. 2001; 47:624-30.
Francis R J, Sharma S K, Springer C, et al. A phase I trial of antibody directed enzyme prodrug therapy (ADEPT) in patients with advanced colorectal carcinoma or other CEA producing tumours. Br J. Cancer. 2002; 87:600-7.
Granowska M, Britton K E, Mather S J, et al. Radioimmunoscintigraphy with technetium-99m labelled monoclonal antibody, 1A3, in colorectal cancer. Eur J Nucl Med. 1993; 20:690-8.
Hammarstrom S. The carcinoembryonic antigen (CEA) family: structures, suggested functions and expression in normal and malignant tissues. Semin Cancer Biol. 1999; 9:67-81.
Hance K W, Zeytin H E, Greiner J W. Mouse models expressing human carcinoembryonic antigen (CEA) as a transgene: evaluation of CEA-based cancer vaccines. Mutat Res. 2005; 576:132-54.
Locker G Y, Hamilton S, Harris J, et al. ASCO 2006 update of recommendations for the use of tumor markers in gastrointestinal cancer. J Clin Oncol. 2006; 24:5313-27.
Mayer A, Tsiompanou E, O'Malley D, et al. Radioimmunoguided surgery in colorectal cancer using a genetically engineered anti-CEA single-chain Fv antibody. Clin Cancer Res. 2000; 6:1711-9.
Rother, M. Carcinoembryonic antigen monitoring for early detection of asymptomatic incurable metastatic colorectal cancer. J Clin Oncol. 2007; 25:1293-4. Author reply on p1294.
Sanders D S, Evans A T, Allen C A, et al. Classification of CEA-related positivity in primary and metastatic malignant melanoma. J. Pathol. 1994; 172:343-8.
Wilkins D, A Mayer, S Sharma, et al. Evidence of efficacy of antibody directed enzyme prodrug therapy (ADEPT) in a Phase I trial in patients with advanced carcinoma. AACR Meeting Abstracts, April 2008: LB-200.
Zhou H, Fuks A, Alcaraz G, Bolling T J, Stanners C P. Homophilic adhesion between Ig superfamily carcinoembryonic antigen molecules involves double reciprocal bonds. J Cell Biol 1993; 122:951-60.

Sequences

SEQ ID NO: 1 - Short form human CEA (including pro-sequences)
MESPSAPPHRWCIPWQRLLLTASLLTFWNPPTTAKLTIESTPFNVAEGKEVLLLVHNLPQ
HLFGYSWYKGERVDGNRQIIGYVIGTQQATPGPAYSGREIIYPNASLLIQNIIQNE/KLSV
DHSDPVILNVLYGPDDPTISPSYTYYRPGVNLSLSCHAASNPPAQYSWLIDGNIQQHTQE
LFISNITEKNSGLYTCQANNSASGHSRTTVKTITVSAELPKPSISSNNSKPVEDKDAVAFT
CEPEAQNTTYLWWVNGQSLPVSPRLQLSNGNRTLTLFNVTRNDARAYVCGIQNSVSAN
RSDPVTLDVLYGPDTPIISPPDSSYLSGANLNLSCHSASNPSPQYSWRINGIPQQHTQVLFI
AKITPNNNGTYACFVSNLATGRNNSIVKSITVSASGTSPGLSAGATVGIMIGVLVGVALI
Note that the bolded and underlined position denotes a
polymorphism - such that the residue at that position may
be either an E or a K (but not both).
SEQ ID NO: 2 - Mature full-length CEA
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELPKPSISSNNSKP
VEDKDAVAFTCEPETQDATYLWWVNNQSLPVSPRLQLSNGNRTLTLFNVTRNDTASYK
CETQNPVSARRSDSVILNVLYGPDAPTISPLNTSYRSGENLNLSCHAASNPPAQYSWFVN
GTFQQSTQELFIPNITVNNSGSYTCQAHNSDTGLNRTTVTTITVYAEPPKPFITSNNSNPV
EDEDAVALTCEPEIQNTTYLWWVNNQSLPVSPRLQLSNDNRTLTLLSVTRNDVGPYECG
IQNE/KLSVDHSDPVILNVLYGPDDPTISPSYTYYRPGVNLSLSCHAASNPPAQYSWLID
GNIQQHTQELFISNITEKNSGLYTCQANNSASGHSRTTVKTITVSAELPKPSISSNNSKPVE
DKDAVAFTCEPEAQNTTYLWWVNGQSLPVSPRLQLSNGNRTLTLFNVTRNDARAYVC
GIQNSVSANRSDPVTLDVLYGPDTPIISPPDSSYLSGANLNLSCHSASNPSPQYSWRINGIP
QQHTQVLFIAKITPNNNGTYACFVSNLATGRNNSIVKSITVSASGTSPGLSA
Note that the bolded and underlined position denotes a
polymorphism - such that the residue at that position may
be either an E or a K (but not both).
SEQ ID NO: 3 - CEA deletion mutant Del1-3
KLTIESTPFNVAEGKEVLLLVHNLPQHLFGYSWYKGERVDGNRQIIGYVIGTQQATPGPA
YSGREIIYPNASLLIQNIIQNDLYGPDDPTISPSYTYYRPGVNLSLSCHAASNPPAQYSWLI
DGNIQQHTQELFISNITEKNSGLYTCQANNSASGHSRTTVKTITVSAELPKPSISSNNSKPV
EDKDAVAFTCEPEAQNTTYLWWVNGQSLPVSPRLQLSNGNRTLTLFNVTRNDARAYVC
GIQNSVSANRSDPVTLDVLYGPDTPIISPPDSSYLSGANLNLSCHSASNPSPQYSWRINGIP
QQHTQVLFIAKITPNNNGTYACFVSNLATGRNNSIVKSITVSASGTSPGLSA
SEQ ID NO: 4 - CEA deletion mutant Del1
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELVLYGPDAPTISP
LNTSYRSGENLNLSCHAASNPPAQYSWFVNGTFQQSTQELFIPNITVNNSGSYTCQAHNS
DTGLNRTTVTTITVYAEPPKPFITSNNSNPVEDEDAVALTCEPEIQNTTYLWWVNNQSLP
VSPRLQLSNDNRTLTLLSVTRNDVGPYECGIQNELSVDHSDPVILNVLYGPDDPTISPSYT
YYRPGVNLSLSCHAASNPPAQYSWLIDGNIQQHTQELFISNITEKNSGLYTCQANNSASG
HSRTTVKTITVSAELPKPSISSNNSKPVEDKDAVAFTCEPEAQNTTYLWWVNGQSLPVSP
RLQLSNGNRTLTLFNVTRNDARAYVCGIQNSVSANRSDPVTLDVLYGPDTPIISPPDSSY
LSGANLNLSCHSASNPSPQYSWRINGIPQQHTQVLFIAKITPNNNGTYACFVSNLATGRN
NSIVKSITVSASGTSPGLSA
SEQ ID NO: 5 - CEA deletion mutant Del2
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELPKPSISSNNSKP
VEDKDAVAFTCEPETQDATYLWWVNNQSLPVSPRLQLSNGNRTLTLFNVTRNDTASYK
CETQNPVSARRSDSVILNITVYAEPPKPFITSNNSNPVEDEDAVALTCEPEIQNTTYLWW
VNNQSLPVSPRLQLSNDNRTLTLLSVTRNDVGPYECGIQNELSVDHSDPVILNVLYGPDD
PTISPSYTYYRPGVNLSLSCHAASNPPAQYSWLIDGNIQQHTQELFISNITEKNSGLYTCQ
ANNSASGHSRTTVKTITVSAELPKPSISSNNSKPVEDKDAVAFTCEPEAQNTTYLWWVN
GQSLPVSPRLQLSNGNRTLTLFNVTRNDARAYVCGIQNSVSANRSDPVTLDVLYGPDTPI
ISPPDSSYLSGANLNLSCHSASNPSPQYSWRINGIPQQHTQVLFIAKITPNNNGTYACFVS
NLATGRNNSIVKSITVSASGTSPGLSA
SEQ ID NO: 6 - CEA deletion mutant Del3
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELPKPSISSNNSKP
VEDKDAVAFTCEPETQDATYLWWVNNQSLPVSPRLQLSNGNRTLTLFNVTRNDTASYK
CETQNPVSARRSDSVILNVLYGPDAPTISPLNTSYRSGENLNLSCHAASNPPAQYSWFVN
GTFQQSTQELFIPNITVNNSGSYTCQAHNSDTGLNRTTVTTITVYAEPPTISPSYTYYRPG
VNLSLSCHAASNPPAQYSWLIDGNIQQHTQELFISNITEKNSGLYTCQANNSASGHSRTT
VKTITVSAELPKPSISSNNSKPVEDKDAVAFTCEPEAQNTTYLWWVNGQSLPVSPRLQLS
NGNRTLTLFNVTRNDARAYVCGIQNSVSANRSDPVTLDVLYGPDTPIISPPDSSYLSGAN
LNLSCHSASNPSPQYSWRINGIPQQHTQVLFIAKITPNNNGTYACFVSNLATGRNNSIVKS
ITVSASGTSPGLSA
SEQ ID NO: 7 - CEA deletion mutant Del4-5
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELPKPSISSNNSKP
VEDKDAVAFTCEPETQDATYLWWVNNQSLPVSPRLQLSNGNRTLTLFNVTRNDTASYK
CETQNPVSARRSDSVILNVLYGPDAPTISPLNTSYRSGENLNLSCHAASNPPAQYSWFVN
GTFQQSTQELFIPNITVNNSGSYTCQAHNSDTGLNRTTVTTITVYAEPPKPFITSNNSNPV
EDEDAVALTCEPEIQNTTYLWWVNNQSLPVSPRLQLSNDNRTLTLLSVTRNDVGPYECG
IQNELSVDHSDPVILNVLYGPDTPIISPPDSSYLSGANLNLSCHSASNPSPQYSWRINGIPQ
QHTQVLFIAKITPNNNGTYACFVSNLATGRNNSIVKSITVSASGTSPGLSA
SEQ ID NO: 8 - CEA truncated mutant IgC3_GPI
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELKPFITSNNSNPV
EDEDAVALTCEPEIQNTTYLWWVNNQSLPVSPRLQLSNDNRTLTLLSVTRNDVGPYECG
IQNELSVDHSDPVILNVLYGPDDASGTSPGLSA
SEQ ID NO: 9 - CEA truncated mutant IgC5_GPI
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELKPSISSNNSKPV
EDKDAVAFTCEPEAQNTTYLWWVNGQSLPVSPRLQLSNGNRTLTLFNVTRNDARAYVC
GIQNSVSANRSDPVTLDVLYGPASGTSPGLSA
SEQ ID NO: 10 - Full-length human CEA (including pro-sequences)
MESPSAPPHRWCIPWQRLLLTASLLTFWNPPTTAKLTIESTPFNVAEGKEVLLLVHNLPQ
HLFGYSWYKGERVDGNRQIIGYVIGTQQATPGPAYSGREIIYPNASLLIQNIIQNDTGFYT
LHVIKSDLVNEEATGQFRVYPELPKPSISSNNSKPVEDKDAVAFTCEPETQDATYLWWV
NNQSLPVSPRLQLSNGNRTLTLFNVTRNDTASYKCETQNPVSARRSDSVILNVLYGPDA
PTISPLNTSYRSGENLNLSCHAASNPPAQYSWFVNGTFQQSTQELFIPNITVNNSGSYTCQ
AHNSDTGLNRTTVTTITVYAEPPKPFITSNNSNPVEDEDAVALTCEPEIQNTTYLWWVNN
QSLPVSPRLQLSNDNRTLTLLSVTRNDVGPYECGIQNE/KLSVDHSDPVILNVLYGPDD
PTISPSYTYYRPGVNLSLSCHAASNPPAQYSWLIDGNIQQHTQELFISNITEKNSGLYTCQ
ANNSASGHSRTTVKTITVSAELPKPSISSNNSKPVEDKDAVAFTCEPEAQNTTYLWWVN
GQSLPVSPRLQLSNGNRTLTLFNVTRNDARAYVCGIQNSVSANRSDPVTLDVLYGPDTPI
ISPPDSSYLSGANLNLSCHSASNPSPQYSWRINGIPQQHTQVLFIAKITPNNNGTYACFVS
NLATGRNNSIVKSITVSASGTSPGLSAGATVGIMIGVLVGVALI
Note that the bolded and underlined position denotes a
polymorphism - such that the residue at that position may
be either an E or a K (but not both).

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1-60. (canceled)

61. A method of determining the presence of a carcinoembryonic antigen (CEA)-expressing cancer in a subject comprising:
obtaining a sample from the subject;
detecting a concentration of full-length CEA protein or RNA in the sample;
detecting a concentration of short form CEA protein or RNA in the sample;
determining a ratio of full-length CEA protein or RNA concentration to short form CEA protein or RNA concentration;
wherein a ratio of full-length CEA protein or RNA concentration to short form protein or RNA concentration higher than one is indicative of the presence of a CEA-expressing cancer in the subject.

62. The method of claim 61, wherein detecting a concentration of full-length CEA protein comprises contacting the sample with an antibody, an antigen binding fragment or an immunoglobulin-like molecule that immunospecifically binds to full-length CEA protein but does not immunospecifically bind to short form CEA protein; wherein the antibody is not A5B7.

63. The method of claim 62, wherein the antibody, antigen binding fragment or immunoglobulin-like molecule immunospecifically binds to a protein comprising the amino acid sequence of SEQ ID NO:2, but does not immunospecifically bind to a protein comprising the amino acid sequence of SEQ ID NO:1.

64. The method of claim 62, wherein the antibody, antigen binding fragment, or immunoglobulin-like molecule comprises an amino acid sequence selected from the amino acid sequences set forth in SEQ ID NO:28 to SEQ ID NO:44 and SEQ ID NO:46 to SEQ ID NO:51.

65. The method of claim 62, wherein the antibody, antigen binding fragment or immunoglobulin-like molecule that immunospecifically binds to full-length CEA protein but does not immunospecifically bind to short form CEA protein is a monoclonal antibody.

66. The method of claim 65, wherein the monoclonal antibody is a chimeric antibody, a humanized antibody, or a fully human antibody.

67. The method of claim 61, wherein detecting a concentration of short form CEA protein comprises contacting the sample with an antibody, an antigen binding fragment or an immunoglobulin-like molecule that immunospecifically binds to short form CEA protein but does not immunospecifically bind to full-length CEA protein.

68. The method of claim 67, wherein the antibody, antigen binding fragment or immunoglobulin-like molecule immunospecifically binds to a protein comprising the amino acid sequence of SEQ ID NO: 1.

69. The method of claim 61, wherein detecting of a full-length CEA protein or a short form CEA protein is performed using immunohistochemistry.

70. The method of claim 61, wherein detecting a concentration of RNA in the sample is performed using TissueScan Disease Tissue qPCR arrays.

71. The method of claim 61, wherein said subject is a human.

72. The method of claim 61, wherein said sample is chosen from whole blood, serum, plasma, saliva, urine, feces, seminal plasma, sweat, amniotic fluid, sputum, breast milk, bile, tissue homogenate, and ascites.

73. The method of claim 61, wherein said CEA-expressing cancer is chosen from colon cancer, rectal cancer, pancreatic cancer, esophageal cancer, gastroesophageal cancer, stomach cancer, lung cancer and breast cancer.

74. The method of claim 61, wherein the antibody, an antigen binding fragment or an immunoglobulin-like molecule comprises an amino acid sequence chosen from the amino acid sequences of SEQ ID NOs: 28-44 and 46-51.

75. A purified polypeptide comprising the amino acid sequence set forth in SEQ ID NO:1, or a fragment of the amino acid sequence set forth in SEQ ID NO:1 comprising the consecutive amino acid residues set forth in SEQ ID NO: 11 (NIIQNELSVD) or the consecutive amino acid residues set forth in SEQ ID NO: 12 (NIIQNKLSVD).

76. The purified polypeptide of claim 75, comprising the consecutive amino acid residues set forth in SEQ ID NO: 11 (NIIQNELSVD).

77. The purified polypeptide of claim 75, comprising the consecutive amino acid residues set forth in SEQ ID NO: 12 (NIIQNKLSVD).

78. A method of detecting recurrence of a CEA-expressing cancer comprising:
obtaining a first sample from a subject having a CEA-expressing cancer and susceptible to anti-CEA-treatment, wherein said first sample is obtained after treatment;
detecting in said first sample a post-treatment concentration of full-length CEA protein and of short form CEA protein;
obtaining one or more further samples from said subject, wherein said further sample is obtained at a later point after treatment;
detecting in said further sample a concentration of full-length CEA protein and a concentration of short form CEA protein;
obtaining a ratio of the concentration of full-length CEA protein and of short form CEA protein;
wherein detecting the concentration of short form CEA protein in said one or more further samples above the concentration of short form CEA protein observed in said first sample indicates recurrence of said CEA-expressing cancer.

79. The method of claim 78, wherein detecting concentration of full-length CEA protein and short form protein is performed using immunohistochemistry.

80. The method of claim 78, wherein said treatment comprises use of a therapeutic which includes, at least, a CEA binding portion comprising the amino acid sequence represented in any of SEQ ID NOs: 28-44 and 46-51.
